(19) United States
(12) Patent Application Publication
(10) Pub. No.: US 2024/0327912 A1
(43) Pub. Date: Oct. 3, 2024

(54) METHODS OF IMMUNE CELL ANALYSIS

(71) Applicant: 10x Genomics, Inc., Pleasanton, CA (US)

(72) Inventors: Wyatt James MCDONNELL, Pleasanton, CA (US); Sarah E.B. TAYLOR, Pleasanton, CA (US)

(21) Appl. No.: 18/627,090

(22) Filed: Apr. 4, 2024

Related U.S. Application Data
(63) Continuation of application No. PCT/US2022/077584, filed on Oct. 5, 2022.
(60) Provisional application No. 63/252,551, filed on Oct. 5, 2021.

(51) Int. Cl. C12Q 1/6881; G01N 33/543

(52) U.S. Cl. CPC C12Q 1/6881; G01N 33/54313

(57) ABSTRACT

Provided herein are methods for identification chimeric antigen receptors in immune cells as well as method for identification and characterization of immune cells expressing those chimeric antigen receptors.

### CROSS-REFERENCE TO RELATED APPLICATIONS

The present application is a continuation, filed under 35 U.S.C. § 120, of PCT/US2022/077584, filed Oct. 5, 2022, which claims priority to U.S. Provisional Patent Application Ser. No. 63/252,551, filed on Oct. 5, 2021. The content of the above-referenced application is herein expressly incorporated by reference in its entirety, including any drawings.

### FIELD

The present disclosure relates to methods for identification and characterization of chimeric antigen receptors (CAR) in immune cells as well as identification and characterization of the immune cells themselves.

### BACKGROUND

Chimeric antigen receptor (CAR) T-cell therapy is a promising type of immunotherapy that has recently emerged as an effective treatment for several blood-based cancers. CAR T-cell therapy involves engineering T cells to specifically target and kill cancerous cells. In the process of creating CAR T cells for therapeutic purposes, T cells are extracted from a patient (autologous) or healthy donor (allogenic), engineered to express a receptor that recognizes a specific cancer cell antigen, and infused back into the patient. Since 2017, FDA has approved five CAR T-cell therapies (four of which target CD19) for treating different types of lymphomas, leukemias, and multiple myeloma.

While CAR T-cell therapy has shown to be effective for treating hematological malignancies, challenges remain in the development of this emerging class of therapeutics, from deepening responses in blood-based cancers to expanding efficacy against solid tumors and overcoming toxicity.

Single cell tools have provided valuable insights for addressing some of the biggest challenges that can complicate CAR T-cell therapy, including the persistence and durability of CAR T cells in patients, clonal heterogeneity within a tumor, suppression in the tumor microenvironment, and toxicity, specifically anti-CD19 infusion product-associated toxicities in patients with large B-cell lymphomas.

Heterogeneity in the infusion product is one of the key challenges in developing effective CAR T-cell therapy. As cell therapy infusion products are inherently heterogeneous and thereby composed of different cell types and associated functional states, optimizing the selection of transduced cells post-manufacturing is essential to ensuring a robust and durable response. Historically, scientists have used flow cytometry and bulk RNA sequencing (RNA-seq) to phenotype populations of CAR T cells and endogenous immune cells. However, these strategies are limited with respect to resolution and sensitivity. While sensitive enough to resolve cell populations, flow cytometry offers less complete resolution of cell types and states than single cell analysis due to spectral overlap and operator variability in the identification of cell populations (commonly referred to as “gating”). Inherent to traditional flow cytometric analysis are the subjectivity of manual gating as well as a limit on the number of markers/parameters that can be assessed.

Bulk RNA-seq can measure CAR T-cell construct presence and abundance, but it cannot be used to link these measurements to individual cells and their functional states. Importantly, bulk RNA-seq is poorly suited to identify rare- or any-cell types in a sample, as the data represent the average expression levels of transcripts across all cells in the sample. Inter-sample variation in cell-type compositions confounds differential expression and related analyses. These cell-type compositions are important for understanding the composition of CAR T-cell infusion products. Thus, there is a clear need for highly sensitive product composition analyses of CAR-transduced cellular populations.

### SUMMARY

Provided herein are compositions, methods, partitions, kits, and systems for characterizing immune cells or immune cells expressing chimeric antigen receptors (CARs), e.g., a CAR-T, CAR-M, CAR-NK cell.

In an aspect, provided herein is a method of characterizing immune cells comprising partitioning a reaction mixture into a plurality of partitions. The reaction mixture comprises a population of immune cells, wherein an immune cell of the population of immune cells comprises a plurality of nucleic acid molecules comprising endogenous sequences and an exogenous chimeric antigen receptor (CAR) sequence, the exogenous CAR sequence comprising an scFv encoding portion. The partitioning provides a partition comprising: (i) the immune cell, and (ii) a plurality of nucleic acid barcode molecules comprising a partition-specific barcode sequence. Barcoded nucleic acid molecules are generated, wherein the barcoded nucleic acid molecules comprise: (i) a first barcoded nucleic acid molecule comprising a sequence corresponding to the exogenous sequence or reverse complement thereof, and (ii) a second barcoded nucleic acid molecule comprising a sequence corresponding to an endogenous sequence or reverse complement thereof. A bait capable of hybridizing to the scFv encoding portion of the exogenous CAR sequence is used to provide an enriched subset of barcoded nucleic acid molecules. The first barcoded nucleic acid molecule (or a derivative thereof) from the enriched subset and the second barcoded nucleic acid molecule (or derivative thereof) are then sequenced to identify the first barcoded nucleic acid molecule and the second barcoded nucleic acid molecule as originating from the immune cell. Such method can be useful for characterizing an infusion product, e.g., with a threshold of sensitivity of 0.0249%, and may be useful for resolving heterogeneity in an infusion product.

In an aspect, provided herein is a method of characterizing immune cells comprising partitioning a reaction mixture into a plurality of partitions. The reaction mixture comprises: (i) a population of immune cells, wherein an immune cell of the population of immune cells comprises a plurality of nucleic acid molecules comprising endogenous sequences and an exogenous sequence, wherein the exogenous sequence comprises an scFv encoding portion. The partitioning provides a partition comprising: (ii) the immune cell, and (iii) a plurality of nucleic acid barcode molecules comprising a partition-specific barcode sequence, and a primer molecule. In the partition, barcoded nucleic acid molecules are generated, wherein the barcoded nucleic acid molecules comprise (i) a first barcoded nucleic acid molecule comprising a sequence corresponding to the exogenous sequence or reverse complement thereof, and (ii) a second barcoded nucleic acid molecule comprising a sequence corresponding to an endogenous sequence or reverse complement thereof. A bait capable of hybridizing to the scFv encoding portion of the exogenous sequence is used to generate an enriched subset of barcoded nucleic acid molecules. The first barcoded nucleic acid molecule (or a derivative thereof) and the second barcoded nucleic acid molecule (or a derivative thereof) are sequenced to identify the first barcoded nucleic acid molecule and the second barcoded nucleic acid molecule as originating from the immune cell. Such method can be useful for characterizing an infusion product, e.g., with a threshold of sensitivity of 0.0249%, and may be useful for resolving heterogeneity in an infusion product.

In another aspect, provided herein is a method of characterizing chimeric antigen receptor-expressing immune cell comprising partitioning a reaction mixture into a plurality of partitions. The reaction mixture comprises: (i) a population of immune cells comprising a chimeric antigen receptor-expressing immune cell, wherein the chimeric antigen receptor-expressing immune cell comprises a plurality of nucleic acid molecules comprising endogenous sequences and an exogenous antigen receptor sequence, wherein the exogenous antigen receptor sequence comprises an scFv encoding portion of the chimeric antigen receptor. The partitioning provides a partition comprising: (i) the chimeric antigen receptor-expressing immune cell, and
(ii) a plurality of nucleic acid barcode molecules comprising a partition-specific barcode sequence, and a primer molecule. In the partition, barcoded nucleic acid molecules are generated. The barcoded nucleic acid molecules comprise: (i) a first barcoded nucleic acid molecule comprising a sequence corresponding to the exogenous sequence or reverse complement thereof, and (ii) a second barcoded nucleic acid molecule comprising a sequence corresponding to the endogenous sequence or reverse complement thereof. A bait capable of hybridizing to the scFv-encoding portion of the exogenous sequence is is used to generate an enriched subset of barcoded nucleic acid molecules and
the first barcoded nucleic acid molecule and the second barcoded nucleic acid molecule are sequenced to identify the first barcoded nucleic acid molecule and the second barcoded nucleic acid molecule as originating from the chimeric antigen receptor-expressing immune cell. Such method can be useful for characterizing an infusion product, e.g., with a threshold of sensitivity of 0.0249%, and may be useful for resolving heterogeneity in an infusion product.

In an aspect, provided herein is a method of resolving heterogeneity in an infusion product comprising partitioning a reaction mixture into a plurality of partitions. The reaction mixture comprises (i) a population of immune cells comprising a chimeric antigen receptor-expressing immune cell, said population being derived from an infusion product, wherein the chimeric antigen receptor-expressing immune cell comprises a plurality of nucleic acid molecules comprising endogenous sequences and an exogenous antigen receptor sequence. The partitioning provides a partition comprising (i) the chimeric antigen receptor-expressing immune cell, and (ii) a plurality of nucleic acid barcode molecules comprising a partition-specific barcode sequence, and a primer molecule. Barcoded nucleic acid molecules are generated in the partition, wherein the barcoded nucleic acid molecules comprise (i) a first barcoded nucleic acid molecule comprising a sequence corresponding to the exogenous sequence or reverse complement thereof, and (ii) a second barcoded nucleic acid molecule comprising a sequence corresponding to the endogenous sequence or reverse complement thereof. The first barcoded nucleic acid molecule and the second barcoded nucleic acid molecule are sequenced to resolve the heterogeneity in the infusion product.

In another aspect, provided herein is a method of characterizing an infusion product with a threshold of sensitivity of 0.0249%. The method involves partitioning a reaction mixture into a plurality of partitions, wherein the reaction mixture comprises (i) a population of immune cells comprising a chimeric antigen receptor-expressing immune cell, said population being derived from an infusion product, wherein the chimeric antigen receptor-expressing immune cell comprises a plurality of nucleic acid molecules comprising endogenous sequences and an exogenous antigen receptor sequence. The partitioning provides a partition comprising (i) the chimeric antigen receptor-expressing immune cell, and (ii) a plurality of nucleic acid barcode molecules comprising a partition-specific barcode sequence, and a primer molecule. Barcoded nucleic acid molecules are generated in the partition. The barcoded nucleic acid molecules comprise (i) a first barcoded nucleic acid molecule comprising a sequence corresponding to the exogenous sequence or reverse complement thereof, and (ii) a second barcoded nucleic acid molecule comprising a sequence corresponding to the endogenous sequence or reverse complement thereof. The first barcoded nucleic acid molecule and the second barcoded nucleic are sequences to identify the first barcoded nucleic acid molecule and the second barcoded nucleic acid molecule as originating from the chimeric antigen receptor-expressing immune cell at a threshold of sensitivity of 0.0249%.

In some embodiments, the bait is biotinylated.

In some embodiments, (c) comprises hybridizing the bait to the first barcoded nucleic acid molecule or derivative thereof via the scFv encoding region.

In some embodiments, (c) comprises associating the bait with a set of beads comprising a biotin-binding partner, thereby operatively coupling the first barcoded nucleic acid molecule or derivative thereof with the set of beads. In some embodiments, the biotin-binding partner is selected from the group consisting of streptavidin, avidin, deglycosylated avidin, traptavidin, tamavidin, xenavidin, bradavidin, AVR2 (avidin related protein 2), AVR4 (avidin related protein 4), and variants, mutants, derivatives, and homologs of any thereof.

In some embodiments, (c) comprises removing barcoded nucleic acid molecules or derivatives thereof that are not operatively coupled to the set of beads, thereby providing the enriched subset of barcoded nucleic acid molecules. In some embodiments, the sequencing the second barcoded nucleic acid molecule or derivative thereof comprises sequencing from the removed barcoded nucleic acid molecules or derivatives thereof.

In one embodiment, the method further comprises providing multiple capture sequences capable of hybridizing to different regions of the scFv region of the exogenous sequence.

In one embodiment, the method further comprises providing capture sequences capable of hybridizing to additional sequences associated with a chimeric antigen receptor T cell.

In an aspect, the description provides for a method of characterizing immune cells comprising partitioning a reaction mixture into a plurality of partitions. The reaction mixture comprises a population of immune cells, wherein an immune cell of the population of immune cells comprises a plurality of nucleic acid molecules comprising endogenous sequences and an exogenous sequence. The partitioning provides a partition comprising the immune cell, and a plurality of nucleic acid barcode molecules comprising a partition-specific barcode sequence, and a primer molecule. In the partition, barcoded nucleic acid molecules are generated, wherein the barcoded nucleic acid molecules comprise (i) a first barcoded nucleic acid molecule comprising a sequence corresponding to the exogenous sequence or reverse complement thereof and (ii) a second barcoded nucleic acid molecule comprising a sequence corresponding to an endogenous sequence or reverse complement thereof. The first barcoded nucleic acid molecule and the second barcoded nucleic acid molecule are sequenced to identify the first barcoded nucleic acid molecule and the second barcoded nucleic acid molecule as originating from the immune cell. Such method can be useful for characterizing an infusion product, e.g., with a threshold of sensitivity of 0.0249%, and may be useful for resolving heterogeneity in an infusion product.

In another aspect, the description provides for a method of characterizing chimeric antigen receptor-expressing immune cells comprising partitioning a reaction mixture into a plurality of partitions. The reaction mixture comprises a population of immune cells comprising a chimeric antigen receptor-expressing immune cell, wherein the chimeric antigen receptor-expressing immune cell comprises a plurality of nucleic acid molecules comprising endogenous sequences and an exogenous antigen receptor sequence. The partitioning provides a partition comprising the chimeric antigen receptor-expressing immune cell, and a plurality of nucleic acid barcode molecules comprising a partition-specific barcode sequence, and a primer molecule. In the partition, barcoded nucleic acid molecules are generated. The barcoded nucleic acid molecules comprise a first barcoded nucleic acid molecule comprising a sequence corresponding to the exogenous sequence or reverse complement thereof and a second barcoded nucleic acid molecule comprising a sequence corresponding to an endogenous sequence or reverse complement thereof. The first barcoded nucleic acid molecule and the second barcoded nucleic acid molecule are sequenced to identify the first barcoded nucleic acid molecule and the second barcoded nucleic acid molecule as originating from the chimeric antigen receptor-expressing immune cell. Such method can be useful for characterizing an infusion product, e.g., with a threshold of sensitivity of 0.0249%, and may be useful for resolving heterogeneity in an infusion product.

In one embodiment, the population of immune cells comprises a non-transduced chimeric antigen receptor-expressing immune cell.

In one embodiment, the population of immune cells is an ex vivo population of immune cells.

In one embodiment, the immune cells are lymphocytes. In one embodiment, the lymphocytes are CD4+ and CD8+ T cells. In one embodiment, the lymphocytes are CD4+ T cells selected from the group consisting of naïve CD4+ T cells, effector CD4+ T cells, memory CD4+ T cells, and activated CD4+ T cells. In one embodiment, the lymphocytes are CD8+ T cells selected from the group consisting of naïve CD8+ T cells, effector CD8+ T cells, memory CD8+ T cells, CD45RA+ CD8+ T cells, and activated CD8+ T cells. In some embodiments, the immune cells are any immune cell expressing a CAR. In one embodiment, the immune cells are selected from the group consisting of CAR-NK and CAR-M cells.

In one embodiment, the CAR-T cell comprises CD19 single chain variable fragment (scFV), CD28, and CD3-gamma.

In one embodiment, the immune cell or chimeric antigen receptor-expressing immune cell further comprises a labeling agent. In one embodiment, the labeling agent further comprises a reporter oligonucleotide. In one embodiment, the reporter oligonucleotide comprises a reporter sequence.

In one embodiment, the labeling agent further comprises an antigen. In one embodiment, the antigen is configured to couple to a surface receptor of the immune cell. In one embodiment, the surface receptor comprises a single chain variable fragment (scFv). In one embodiment, the scFv is encoded by the exogenous sequence.

In one embodiment, the first and second barcoded molecules further comprise a capture sequence configured to couple to an mRNA or DNA analyte.

In one embodiment, the capture sequence configured to couple to the mRNA or DNA analyte is configured to couple to the mRNA analyte, and wherein the capture sequence configured to couple to the mRNA analyte comprises a polyT sequence.

In one embodiment, the second barcoded nucleic acid molecule comprises a sequence corresponding to an immune receptor. In one embodiment, the method further comprises amplifying sequences corresponding to the immune receptor gene to provide an enriched subset of barcoded nucleic acid molecules. In one embodiment, the method further comprises sequencing the enriched subset of barcoded nucleic acid molecules.

In one embodiment, the sequencing comprises providing a capture sequence capable of hybridizing to the scFv region of the CAR. In one embodiment, the capture sequence capable of hybridizing to the scFv region of the CAR/exogenous sequence is specific to CD19.

In one embodiment, a barcoded nucleic acid molecule further comprises a unique molecular identifier (UMI) sequence.

In one embodiment, the partition is a droplet, a micro-vesicle, a flowcell, a reaction chamber, a reaction compartment, a tube, a well, or a microwell. In one embodiment, the partition is a droplet. In one embodiment, the barcode nucleic acid molecules provided as part of a support.

In one embodiment, the generating comprises hybridizing the primer molecule to the plurality of nucleic acid molecules comprising endogenous sequences and the exogenous sequences. In one embodiment, this aspect further comprises performing a nucleic acid extension reaction to provide the first barcoded nucleic acid molecule and the second barcoded nucleic acid molecule. In one embodiment, the plurality of nucleic acid molecules comprises messenger ribonucleic acid (mRNA) molecules and the nucleic acid extension reaction is a reverse transcription reaction that generates complementary deoxyribonucleic acid (cDNA) molecules from the mRNA molecules. In one embodiment, this aspect further comprises performing a template switching reaction onto a nucleic acid barcode molecule of the plurality of nucleic acid barcode molecules and extending a 3′ end of a cDNA molecule using the nucleic acid barcode molecule as a template to generate a barcoded cDNA molecule comprising a reverse complement of the partition-specific barcode sequence.

In one embodiment, the plurality of nucleic acid barcode molecules comprises from 5′ to 3′, the partition-specific barcode sequence and a template switching sequence. In one embodiment, the template switching sequence is configured to capture a cDNA molecule and facilitate a template switching reaction of the cDNA molecule onto the nucleic acid barcode molecule as a template. In one embodiment, the template switching sequence is a 3′ terminal template switching sequence. In one embodiment, the template switching sequence comprises a poly-guanine (polyG) sequence at said 3′ terminus of said nucleic acid barcode molecule.

In one embodiment, the generating comprises hybridizing the reporter oligonucleotide to a nucleic acid barcode molecule of the plurality of nucleic acid barcode molecules and performing a nucleic acid extension reaction to generate an additional barcoded nucleic acid molecule. In one embodiment, the additional barcoded nucleic acid molecule comprises the partition-specific barcode sequence or reverse complement thereof and the reporter sequence or reverse complement thereof. In one embodiment, this aspect further comprises sequencing the additional barcoded nucleic acid molecule or a derivative generated therefrom, thereby generating sequencing information corresponding to the additional barcoded nucleic acid molecule. In one embodiment, this aspect further comprises using the sequencing information to associate the labeling agent with the immune cell or the chimeric antigen receptor-expressing immune cell. In one embodiment, the labeling agent is an antigen, the reporter sequence identifies the antigen, and the sequence information associates the antigen with the surface receptor of the immune cell or the chimeric antigen receptor-expressing immune cell.

In one embodiment, the nucleic acid barcode molecule further comprises a functional sequence.

In one embodiment, the plurality of nucleic acid barcode molecules is provided as part of a support. In one embodiment, the support is a bead. In one embodiment, the bead is a gel bead.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows the workflow of single cell profiling with targeted gene expression analysis. Donor anti-CD19 CAR T cells were sampled at 100% T cells, then mixed/spiked into PBMCs from an unrelated individual at 0.1%, 1%, 5%, and 10% of the sample. Targeted scRNA-seq using the Human Immunology Panel and an add-on custom bait set targeting the scFv region of the CAR vector was performed on 5,000 recovered cells.

FIG. 2A demonstrates identification and characterization of CAR T cells using single cell gene expression analysis. Flow analysis of null-transduced cells is shown. Donor PBMCs were transduced with a CAR-null vector lacking the anti-CD19 CAR and the accompanying FLAG-tag. Data presented here show that no detectable anti-CD19 CAR signal is present in null-transduced cells from the donor. X axis: anti-FLAG-tag fluorescence. Y axis: anti-CD3 fluorescence.

FIG. 2B demonstrates identification and characterization of CAR T cells using single cell gene expression analysis. Flow analysis of transduced cells showing gating is depicted. Donor PBMCs were transduced with the CAR-positive vector containing the anti-CD19 CAR and the accompanying FLAG-tag. Data presented here show efficient transduction of donor T cells with the anti-CD19 CAR vector. X axis: anti-FLAG-tag fluorescence. Y axis: anti-CD3 fluorescence.

FIG. 2C demonstrates identification and characterization of CAR T cells using single cell gene expression analysis. A graphical representation of results from a killing assay are shown. Incubation of CD19 presenting HeLa cells with a) no additional cells, b) non-transduced T cells, and c) CAR+ T cells was performed. This data shows that the CAR+ T cells kill the transduced HeLa cells quickly due to their targeting capacity.

FIG. 2D demonstrates identification and characterization of CAR T cells using single cell gene expression analysis. Detection of anti-CD19 expression based on targeting baits in a sample of 100% CAR T cells is shown. Approximately 42% of cells show expression of the genes.

FIG. 2E demonstrates identification and characterization of CAR T cells using single cell gene expression analysis. CAR+ cell subclustering with cell type classification based on gene expression is shown.

FIG. 3A shows recoveries of CAR+ cells in a titration series. A titration experiment to demonstrate the sensitivity of the targeted gene expression solution and its ability to detect CAR-transduced cells at very low frequencies in samples was performed. CAR T cells were spiked into PBMCs at a concentration of 0.1%, and the population of identified CAR T cells is shown.

FIG. 3B shows recoveries of CAR+ cells in a titration series. A titration experiment to demonstrate the sensitivity of the targeted gene expression solution and its ability to detect CAR-transduced cells at very low frequencies in samples was performed. CAR T cells were spiked into PBMCs at a concentration of 1%, and the population of identified CAR T cells is shown.

FIG. 3C shows recoveries of CAR+ cells in a titration series. A titration experiment to demonstrate the sensitivity of the targeted gene expression solution and its ability to detect CAR-transduced cells at very low frequencies in samples was performed. CAR T cells were spiked into PBMCs at a concentration of 5%, and the population of identified CAR T cells is shown.

FIG. 3D shows recoveries of CAR+ cells in a titration series. A titration experiment to demonstrate the sensitivity of the targeted gene expression solution and its ability to detect CAR-transduced cells at very low frequencies in samples was performed. CAR T cells were spiked into PBMCs at a concentration of 10%, and the population of identified CAR T cells is shown.

FIG. 3E shows expected CAR+ cells based on expression levels from both flow cytometric (63% positive) and gene expression (42% positive) analysis compared to the actual expression levels seen in the titration series.

FIG. 4 shows an exemplary microfluidic channel structure for partitioning individual biological particles in accordance with some embodiments of the disclosure.

FIG. 5 shows an exemplary microfluidic channel structure for the controlled partitioning of beads into discrete droplets.

FIG. 6 shows an exemplary barcode carrying bead.

FIG. 7 illustrates another example of a barcode carrying bead.

FIG. 8A-B schematically illustrates (A) an example microwell array and (B) exemplary workflows for processing nucleic acid molecules using a microwell array.

FIG. 9 schematically illustrates examples of labelling agents.

FIG. 10 depicts an example of a barcode carrying bead.

FIGS. 11A, 11B and 11C schematically depict an example workflow for processing nucleic acid molecules.

FIG. 12 shows an exemplary microfluidic channel structure for delivering barcode carrying beads to droplets.

### DETAILED DESCRIPTION

The present disclosure generally relates to, inter alia, compositions, methods, partitions, kits, and systems for characterizing immune cells or immune cells expressing a chimeric antigen receptor, e.g., a CAR-T, CAR-M, CAR-NK cell.

In the following detailed description, reference is made to the accompanying drawings, which form a part hereof. In the drawings, similar symbols generally identify similar components, unless context dictates otherwise. The illustrative alternatives described in the detailed description, drawings, and claims are not meant to be limiting. Other alternatives may be used and other changes may be made without departing from the spirit or scope of the subject matter presented here. It will be readily understood that the aspects, as generally described herein, and illustrated in the Figures, can be arranged, substituted, combined, and designed in a wide variety of different configurations, all of which are explicitly contemplated and make part of this application.

Unless otherwise defined, all terms of art, notations, and other scientific terms or terminology used herein are intended to have the meanings commonly understood by those of skill in the art to which this application pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The practice of the present invention will employ, unless otherwise indicated, conventional techniques of molecular biology, microbiology, cell biology, biochemistry, nucleic acid chemistry, and immunology, which are well known to those skilled in the art. Such techniques are explained fully in the literature, such as Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferré, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference

### Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B”.

As used herein, a “subject” or an “individual” includes animals, such as human (e.g., human individuals) and non-human animals. The term “non-human animals” includes all vertebrates, e.g., mammals, e.g., rodents, e.g., mice, non-human primates, and other mammals, such as e.g., rat, mouse, cat, dog, cow, pig, sheep, horse, goat, rabbit; and non-mammals, such as amphibians, reptiles, etc. A subject can be a healthy individual, an asymptomatic individual, an individual that has or is suspected of having a disease (e.g., cancer or infection), an individual having a pre-disposition to a disease, an individual that is in need of therapy for a disease, or an individual who has recovered from a disease. In any event, the subject may have been exposed to an antigen characteristic of the disease, such as an antigen capable of producing an antibody immune response associated with the disease.

The term “barcode” is used herein to refer to a label, or identifier, that conveys or is capable of conveying information (e.g., information about an analyte in a sample, a bead, and/or a nucleic acid barcode molecule). A barcode can be part of an analyte or nucleic acid barcode molecule, or independent of an analyte or nucleic acid barcode molecule. A barcode can be attached to an analyte or nucleic acid barcode molecule in a reversible or irreversible manner. A particular barcode can be unique relative to other barcodes. Barcodes can have a variety of different formats. For example, barcodes can include polynucleotide barcodes, random nucleic acid and/or amino acid sequences, and synthetic nucleic acid and/or amino acid sequences. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for or facilitates identification and/or quantification of individual sequencing-reads. In some embodiments, a barcode can be configured for use as a fluorescent barcode. For example, in some embodiments, a barcode can be configured for hybridization to fluorescently labeled oligonucleotide probes. Barcodes can be configured to spatially resolve molecular components found in biological samples, for example, at single-cell resolution (e.g., a barcode can be or can include a “spatial barcode”). In some embodiments, a barcode includes two or more sub-barcodes that together function as a single barcode. For example, a polynucleotide barcode can include two or more polynucleotide sequences (e.g., sub-barcodes). In some embodiments, the two or more sub-barcodes are separated by one or more non-barcode sequences. In some embodiments, the two or more sub-barcodes are not separated by non-barcode sequences.

In some embodiments, a barcode can include one or more unique molecular identifiers (UMIs). Generally, a unique molecular identifier is a contiguous nucleic acid segment or two or more non-contiguous nucleic acid segments that function as a label or identifier for a particular analyte, or for a nucleic acid barcode molecule that binds a particular analyte (e.g., mRNA) via the capture sequence.

A UMI can include one or more specific polynucleotides sequences, one or more random nucleic acid and/or amino acid sequences, and/or one or more synthetic nucleic acid and/or amino acid sequences. In some embodiments, the UMI is a nucleic acid sequence that does not substantially hybridize to analyte nucleic acid molecules in a biological sample. In some embodiments, the UMI has less than 80% sequence identity (e.g., less than 70%, 60%, 50%, or less than 40% sequence identity) to the nucleic acid sequences across a substantial part (e.g., 80% or more) of the nucleic acid molecules in the biological sample. These nucleotides can be completely contiguous, i.e., in a single stretch of adjacent nucleotides, or they can be separated into two or more separate subsequences that are separated by 1 or more nucleotides.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value. In some embodiments, the term “about” indicates the designated value±up to 10%, up to ±5%, or up to ±1%.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, the use of these terms in the specification does not by itself connote any required priority, precedence, or order.

It is understood that aspects and embodiments of the disclosure described herein include “comprising”, “consisting”, and “consisting essentially of” aspects and embodiments. As used herein, “comprising” is synonymous with “including”, “containing”, or “characterized by”, and is inclusive or open-ended and does not exclude additional, unrecited elements or method steps. As used herein, “consisting of” excludes any elements, steps, or ingredients not specified in the claimed composition or method. As used herein, “consisting essentially of” does not exclude materials or steps that do not materially affect the basic and novel characteristics of the claimed composition or method. Any recitation herein of the term “comprising”, particularly in a description of components of a composition or in a description of steps of a method, is understood to encompass those compositions and methods consisting essentially of and consisting of the recited components or steps.

### Methods of the Disclosure

Methods for Characterizing Immune Cells and/or Infusion Products

As described in more detail below, some aspects of the disclosure relate to new approaches and methods for characterizing immune cells or immune cells expressing a chimeric antigen receptor. The methods for the characterization of the immune cells and chimeric antigen receptor-expressing immune cells may characterize both the specific cell type and cell activation state as well as identification of the incorporation of a CAR into these various cell types and states. Some aspects of the disclosure also relate to methods for resolving heterogeneity in an infusion product and characterizing an infusion product with a high threshold of sensitivity (i.e., at least 0.0249%).

In contrast to traditional methods, the combination of single cell RNA-seq (scRNA-seq) and single cell transcriptomic analysis provides the resolution and sensitivity needed to detect and quantify CAR incorporation within a heterogeneous group of cell types and functional states. Custom targeted panels further enables the detection and deep phenotyping of defined transcripts, thereby enabling detection of CAR T cells within a large population of single cells. The methods described herein enable product composition analyses of CAR-transduced cellular populations at higher resolution and sensitivity than currently employed technologies, such as flow cytometry. The methods described herein can detect constructs with increased sensitivity and precision, and they can be used to deeply characterize immune phenotypes, including cell type and functional state. This enhanced phenotyping allows researchers to resolve inherent heterogeneity in the infusion product, offering insight into the effect of starting material and manufacturing conditions on the durability and efficacy of CAR T-cell therapy.

In the methods provided herein, a reaction mixture can be partitioned into a plurality of partitions. The partitioning of the reaction mixture can also be referred to as the compartmentalization or depositing of the reaction mixture into discrete compartments or partitions, where each partition maintains separation of its own contents from the contents of other partitions.

The reaction mixture, which may partitioned, can include: (i) a population of immune cells, wherein an immune cell of the population of immune cells comprises a plurality of nucleic acid molecules comprising endogenous sequences and an exogenous sequence. In one instance, the exogenous sequence comprises a sequence of a single chain variable fragment (an scFv). In some embodiments, the exogenous sequence comprises an scFv-encoding portion. Alternatively, the reaction, which may be partitioned, can include: (i) a population of immune cells comprising a chimeric antigen receptor-expressing immune cell, wherein the chimeric antigen receptor-expressing immune cell comprises a plurality of nucleic acid molecules comprising endogenous sequences and an exogenous antigen receptor sequence. In one instance, the exogenous antigen receptor sequence comprises a sequence of a single chain variable fragment (scFv) of the chimeric antigen receptor. In some embodiments, the exogenous sequence comprises an scFv-encoding portion. Further, the reaction mixture, which may be partitioned, can include: (i) a population of immune cells comprising a chimeric antigen receptor-expressing immune cell, said population being derived from an infusion product, wherein the chimeric antigen receptor-expressing immune cell comprises a plurality of nucleic acid molecules comprising endogenous sequences and an exogenous antigen receptor sequence.

### Immune Cells

Methods described herein can be utilized for sequence analysis of the immune repertoire as well as identification of immune cells that have incorporated a CAR. Analysis of sequence information underlying the immune repertoire can provide a significant improvement in understanding the status and function of the immune system

The population of immune cells in the reaction mixture can comprise a B cell, a monocyte, a natural killer (NK) cell, a natural killer T (NKT) cell, a basophil, an cosinophil, a neutrophil, a dendritic cell, a macrophage, a regulatory T cell, a helper T cell (TH), a cytotoxic T cell (TCTL), or other T cell. In some embodiments, the immune cell is a T lymphocyte. In some embodiments, the cell is a precursor T cell or a T regulatory (Treg) cell. In some embodiments, the cell is a CD34+, CD8+, or a CD4+ cell. In some embodiments, the cell is a CD8+ T cytotoxic lymphocyte cell selected from the group consisting of naïve CD8+ T cells, central memory CD8+ T cells, effector memory CD8+ T cells, CD45RA+CD8+ T cells, activated CD8+ T cells and bulk CD8+ T cells. In some embodiments of the cell, the cell is a CD4+ T helper lymphocyte cell selected from the group consisting of naïve CD4+ T cells, central memory CD4+ T cells, effector memory CD4+ T cells, activated CD4+ T cells, and bulk CD4+ T cells. The population of immune cells in the reaction mixture may also comprise a CAR-T cell. In some embodiments, the population of immune cells comprises a non-transduced CAR-T cell. In some embodiments, the population of immune cells is an ex vivo population of immune cells. The population of immune cells may be obtained from a subject, e.g., a mammal such as a human. If the population of cells is obtained from a subject, the population of cells may include cells obtained from a sample of the subject. The sample of the subject may be obtained by biopsy, core biopsy, needle aspirate, or fine needle aspirate. The sample may be a fluid sample, such as a blood sample, urine sample, or saliva sample. The sample can be a plasma or serum sample,

In some embodiments, the population of immune cells is obtained from an infusion product. Generally, an infusion product comprises autologous peripheral blood mononuclear cells that are collected from a patient in need of a certain treatment. Cells are engineered to express a CAR, for example, activated, and/or expanded using the methods known in the art and then infused back into the patient.

If the population of immune cells in the reaction mixture is obtained from a sample of the subject, the sample may have been processed prior to its inclusion in the reaction mixture. The processing of the sample may include steps such as filtration, selective precipitation, purification, centrifugation, agitation, heating, and/or other processes. For example, a sample may be filtered to remove a contaminant or other materials. In some cases, cells and/or cellular constituents of a sample can be processed to separate and/or sort cells of different types, e.g., to separate T cells from other cell types, including the separation of T cell subpopulations such as memory T cells. A separation process can be a positive selection process, a negative selection process (e.g., removal of one or more cell types and retention of one or more other cell types of interest), and/or a depletion process (e.g., removal of a single cell type from a sample, such as removal of red blood cells from peripheral blood mononuclear cells).

### Chimeric Antigen Receptor Immune Cells

Chimeric antigen receptor immune cells, as used herein, can refer to artificial immune cell receptors, chimeric immune cell receptors, or chimeric immunoreceptors, for example, and encompass engineered receptors that graft an artificial specificity onto a particular immune effector cell. CARs may be employed to impart the specificity of a monoclonal antibody onto an immune cell, thereby allowing a large number of specific immune cells cells to be generated, for example, for use in adoptive cell therapy. In specific embodiments, CARs direct specificity of the cell to a tumor associated antigen, for example. In some embodiments, CARs comprise an intracellular activation domain, a transmembrane domain, and an extracellular domain comprising a tumor associated antigen binding region. In particular aspects, CARs comprise fusions of single-chain variable fragments (scFv) derived from monoclonal antibodies, fused to CD3ζ a transmembrane domain and endodomain.

Exemplary CARs include fusions of short variable fragments (scFv) derived from monoclonal antibodies and fused to the CD3 zeta transmembrane and CD28 domains, or other TCR signaling domains. CAR can target malignant cells, for example, by targeting CD19.

Exemplary chimeric antigen receptor immune cells can include, without limitation, CAR-T cells, CAR-M cells, and CAR-NK cells.

Labeling Immune Cells with Antigens and Barcodes

The immune cell within the reaction mixture, which may be partitioned, can comprise a labeling agent. In some embodiments, the labeling agent may be coupled to a reporter oligonucleotides. In such a reaction mixture, an immune cell of the population of immune cells may be bound to a target antigen coupled to the reporter oligonucleotide. Further, the partitioning of such a reaction mixture, can provide a partition which includes (a) the immune cell or chimeric antigen receptor-expressing immune cell and (b) a plurality of nucleic acid barcode molecules having a partition-specific barcode sequence and a primer molecule.

The target antigen can be any antigen which is configured to couple to a surface receptor of the immune cell (e.g., a CAR of interest). In some embodiments, the surface receptor comprises a single chain variable fragment (scFV). In some embodiments, the scFV is encoded by the exogenous sequence present in the population of immune cells. The target antigen can be an antigen associated with an infectious agent, such as a viral, bacterial, parasitic, protozoal or prion agent. If the target antigen is associated with an infectious agent that is a viral agent, the viral agent can be an influenza virus, a coronavirus, a retrovirus, a rhinovirus, or a sarcoma virus. The viral agent may be severe acute respiratory syndrome coronavirus 1 (SARS-COV-1), a SARS-COV-2, a Middle East respiratory syndrome coronavirus (MERS-COV)), or human immunodeficiency virus (HIV), influenza, respiratory syncytial virus, or Ebola virus. If the target antigen is associated with an infectious agent that is a viral agent, the target antigen can be corona virus spike(S) protein, an influenza hemagglutinin protein, an HIV envelope protein or any other a viral glycoprotein. Further, the target antigen can be associated with a tumor or a cancer. If the target antigen is associated with tumors or cancers, it can be, for example, epidermal growth factor receptor (EGFR), CD38, platelet-derived growth factor receptor (PDGFR) alpha, insulin growth factor receptor (IGFR), CD20, CD19, CD47, or human epidermal growth factor receptor 2 (HER2). In addition, the target antigen can be an immune checkpoint molecule that may or may not be associated with tumors or cancers (e.g., CD38, PD-1, CTLA-4, TIGIT, LAG-3, VISTA, TIM-3), or it may be a cytokine, a GPCR, a cell-based co-stimulatory molecule, a cell-based co-inhibitory molecule, an ion channel, or a growth factor. Further still, the target antigen may be associated with a degenerative condition or disease (e.g., an amyloid protein or a tau protein).

In some embodiments, the target antigen, for which the characterization and/or identification of CAR, having affinity thereto may be desirable, can be a target antigen of a length of at least 20 amino acid residues, at least 40 amino acid residues, at least 60 amino acid residues, at least 80 amino acid residues, at least 100 amino acid residues, at least 200 amino acid residues, at least 300 amino acid residues, at least 400 amino acid residues, at least 500 amino acid residues, at least 600 amino acid residues, at least 700 amino acids, at least 800 amino acid residues, at least 900 amino acid residues, at least 1000 amino acid residues, at least 1100 amino acid residues, at least 1200 amino acid residues, at least 1300 amino acid residues, up to 40 amino acid residues, up to 60 amino acid residues, up to 80 amino acid residues, up to 100 amino acid residues, up to 200 amino acid residues, up to 300 amino acid residues, up to 400 amino acid residues, up to 500 amino acid residues, up to 600 amino acid residues, up to 700 amino acids, up to 800 amino acid residues, up to 900 amino acid residues, up to 1000 amino acid residues, up to 1100 amino acid residues, up to 1200 amino acid residues, or up to 1300 amino acid residues. The target antigen may be an antigen that includes one domain, at least one domain, two domains, at least two domains, three domains, at least three domains, four domains, at least four domains, five domains, at least five domains, six domains, at least six domains, seven domains, at least seven domains, eight domains, at least eight domains, nine domains, at least nine domains, ten domains, at least ten domains, at least thirty domains, at least forty domains, at least fifty domains, at least sixty domains, at least seventy domains, at least eighty domains, at least ninety domains or at least one hundred domains. The target antigen may be an antigen that includes at most two hundred domains, at most 175 domains, at most 150 domains, at most 125 domains, at most 100 domains, at most 75 domains, at most 50 domains, at most 25 domains, at most 20 domains, at most 15 domains, at most 10 domains, or at most 5 domains.

A reporter oligonucleotide, bound to any of an immune cell, can be or include a nucleotide sequence that is specific for the target antigen or immune cell to which it is coupled. The reporter oligonucleotide may include nucleotide sequences including (a) a reporter sequence, e.g., which may be useful to identify the immune cell or chimeric antigen receptor-expressing immune cell which the reporter oligonucleotide is bound. In addition, the reporter oligonucleotide may have a further characteristic in that it may be coupled to a labeling agent. The labeling agent may be coupled to the reporter oligonucleotide via a labeling of the target antigen and/or any fragment thereof, or via a labeling of a nucleotide(s) of the reporter oligonucleotide.

Nucleic acid barcode molecules of the plurality of nucleic acid barcode molecules, included in the partition with the immune cell or chimeric antigen receptor-expressing immune cell, can include a partition-specific barcode sequence. A partition-specific barcode sequence can identify the partition in which the nucleic acid barcode molecule is partitioned. Nucleic acid barcode molecules of the plurality of nucleic acid barcode molecules may further include a capture sequence. A capture sequence may be configured to couple to the handle sequence of a reporter oligonucleotide, e.g., by complementary base pairing. A capture sequence may be configured to couple to an mRNA or a DNA analyte. If the capture sequence is configured to couple to an mRNA analyte, it may include a polyT sequence.

In any of the methods provided herein, once the reaction is partitioned, barcoded nucleic acid molecules, including a first barcoded nucleic acid molecule and a second barcoded nucleic acid molecule, may be generated in the partition. The barcoded nucleic molecules include: (i) a first barcoded nucleic acid molecule comprising a sequence corresponding to the exogenous sequence or reverse complement thereof and (ii) a second barcoded nucleic acid molecule comprising a sequence corresponding to the endogenous sequence or reverse complement thereof. In some embodiments, the second barcoded nucleic acid molecule comprises a sequence corresponding to an immune receptor (e.g., a T cell receptor).

Immune cells express various adaptive immunological receptors relating to immune function, such as T cell receptors and B cell receptors. T cell receptors and B cells receptors play a part in the immune response by specifically recognizing and binding to antigens and aiding in their destruction.

The T cell receptor, or TCR, is a molecule found on the surface of T cells that is generally responsible for recognizing fragments of antigen as peptides bound to major histocompatibility complex (MHC) molecules. The TCR is generally a heterodimer of two chains, each of which is a member of the immunoglobulin superfamily, possessing an N-terminal variable (V) domain, and a C terminal constant domain. In humans, in 95% of T cells the TCR consists of an alpha (a) and beta (P) chain, whereas in 5% of T cells the TCR consists of gamma and delta (y/8) chains. This ratio can change during ontogeny and in diseased states as well as in different species. When the TCR engages with antigenic peptide and MHC (peptide/MHC), the T lymphocyte is activated through signal transduction.

Each of the two chains of a TCR contains multiple copies of gene segments—a variable ‘V gene segment, a diversity ‘D’ gene segment, and a joining ‘J’ gene segment. The TCR alpha chain is generated by recombination of V and J segments, while the beta chain is generated by recombination of V, D, and J segments. Similarly, generation of the TCR gamma chain involves recombination of V and J gene segments, while generation of the TCR delta chain occurs by recombination of V, D, and J gene segments. The intersection of these specific regions (V and J for the alpha or gamma chain, or V, D and J for the beta or delta chain) corresponds to the CDR3 region that is important for antigen-MHC recognition. Complementarity determining regions (e.g., CDR1, CDR2, and CDR3), or hypervariable regions, are sequences in the variable domains of antigen receptors (e.g., T cell receptor and immunoglobulin) that can complement an antigen. Most of the diversity of CDRs is found in CDR3, with the diversity being generated by somatic recombination events during the development of T lymphocytes. A unique nucleotide sequence that arises during the gene arrangement process can be referred to as a clonotype.

It will be understood that any of the barcoded nucleic acid molecules may further include a unique molecular identifier (UMI). The UMI may be a sequence that originating from a reporter oligonucleotide or a nucleic acid barcode molecule.

In methods related to resolving the heterogeneity in an infusion product, the number of CAR transcripts per cell can be determined based on a quantity/number of unique molecular identifiers (UMIs) present in each cell. By way of example, this would allow the determination of whether or not a potential infusion product contained cells expressing an acceptable amount of CARs.

### Targeted Gene Expression Analysis and Sequencing

The methods provided herein include subsequent operations following the generation of barcoded nucleic acid molecules, e.g., in the partition. These subsequent operations may further include amplification of the barcoded nucleic acid molecules. The amplification of the barcoded nucleic acid molecules may optionally be performed using primers that add additional functional sequences to the barcoded nucleic acid molecules. These subsequent operations may include further processing (e.g., shearing, ligation of functional sequences, and subsequent amplification (e.g., via PCR)). These operations can occur in bulk (e.g., outside the partition). In the case where a partition is a droplet in an emulsion, the emulsion can be broken and the contents of the droplet pooled for additional operations. These subsequent operations may include determining sequences of the generated barcoded nucleic acid molecules.

An exemplary operation following the generation of barcoded nucleic acid molecules, e.g., in the partition is the use of targeted gene expression for library enrichment. This is described in Targeted Gene Expression-Single Cell User Guide, 10× Genomics, August 2021, which is hereby incorporated by reference in its entirety. Briefly, following the generation of barcoded nucleic acid molecules in the partition, target enrichment is achieved by using gene-specific, biotinylated baits. These baits are hybridized to their complement in the library, bound to streptavidin beads, and washed to remove non-targeted library molecules. The bead-bound, targeted library fragments are amplified to produce sequencing-ready libraries. Biotinylated baits are provided as pre-designed panels targeting human genes.

In some embodiments, if the partition comprises a chimeric antigen receptor-expressing immune cell, the sequencing of the first barcoded nucleic acid molecule and the second nucleic acid molecule comprises providing a capture sequence (i.e., bait) capable of hybridizing to the scFV region of the CAR. In some embodiments, the bait is used to generate an enriched subset of barcoded nucleic acid molecules. In some embodiments, the capture sequence (i.e., bait) is complementary to a portion of the scFv encoding sequence. In some embodiments, the capture sequence (i.e., bait) hybridizes to the first barcoded nucleic acid molecule or derivative thereof via the scFv encoding region. In some embodiments, the capture sequence (i.e., bait) capable of hybridizing to the scFV region of the CAR is specific to CD19.

In some embodiments, the capture sequence (i.e., bait) is biotinylated. In some embodiments, the bait can also be associated with a set of beads comprising a biotin-binding partner, thereby operatively coupling the first barcoded nucleic acid molecule or derivative thereof with the set of beads. Exemplary biotin-binding partners included, without limitation, those selected from the group consisting of streptavidin, avidin, deglycosylated avidin, traptavidin, tamavidin, xenavidin, bradavidin, AVR2 (avidin related protein 2), AVR4 (avidin related protein 4), and variants, mutants, derivatives, and homologs of any thereof.

In some embodiments, to generate an enriched subset of barcoded nucleic acid molecules, barcoded nucleic acid molecules or derivatives thereof that are not operatively coupled to the set of beads can be removed. This can be done, for example, by performing one or more washing steps to remove those barcoded nucleic acid molecules or derivatives thereof that are not operatively coupled to the set of beads.

In some embodiments, multiple capture sequences capable of hybridizing to different regions of the scFv region are provided. In some embodiments, at least 2, at least 3, at least 4, at least 5, or at least 6 capture sequences capable of hybridizing to different regions of the scFv region are provided.

In some embodiments, capture sequences capable of hybridizing to additional sequences associated with a chimeric antigen receptor expressing immune cell can be further provided. Exemplary sequences include, without limitation, sequences are selected to target specific genes associated with CAR-expressing cells. In some embodiments, the specific genes associated with CAR-expressing cells are selected from the group consisting of ABCA1, ACP5, ADAMDECI, ADM, AK8, ANKRD22, ARRDC4, BCL7A, C15orf48, CD226, CDA, CEACAM3, CIITA, CLEC12A, CRABP2, CSF2RA, CST3, CSTA, CSTB, CTSB, CYP1B1, ENPP3, EPB41L3, EREG, FBP1, FGR, FTL, GGT5, GPNMB, HAVCR1, HLX, ID3, IGSF6, IL411, INHBA, KYNU, LY2, MAFB, MMP14, MNDA, Ms4A7, NCF1, OSCAR, PDLIM4, PILRA, PLA2G7, PLBD1, PTAFR, NECTIN2, RAB13, RBM47, RC3H2, RC3H1, SDC2, SLAMF8, SLC15A3, SLCIA3, SLC43A2, SOD2, SPI1, TCF3, TFEC, TGM2, TMEM176A, TMEM176B, TNFAIP2, TNFAIP6, TNS3, TXN, TYROBP, and ZNF385A. In some embodiments, multiple capture sequences can be used to target a specific gene associated with CAR-expressing cells.

When targeted gene expression for library enrichment is used as described above, prior to sequencing, hybridized library molecules bound to, e.g., streptavidin beads, are amplified with P5 and P7 primers.

In the methods described herein, the subsequent sequencing of the first and second barcoded nucleic acid molecules identifies these barcoded nucleic acid molecules as originating from the immune cell or chimeric antigen receptor-expressing immune cell. This allows detection of CAR construct presence with increased sensitivity and precision, and/or characterization immune phenotypes, including cell type and functional state. In some embodiments, sequencing the second barcoded nucleic acid molecule or derivative thereof comprises sequencing from the removed barcoded nucleic acid molecules or derivatives thereof.

Sequencing may be by performed by any of a variety of approaches, systems, or techniques, including next-generation sequencing (NGS) methods. Sequencing may be performed using nucleic acid amplification, polymerase chain reaction (PCR) (e.g., digital PCR and droplet digital PCR (ddPCR), quantitative PCR, real time PCR, multiplex PCR, PCR-based singleplex methods, emulsion PCR), and/or isothermal amplification. Non-limiting examples of nucleic acid sequencing methods include Maxam-Gilbert sequencing and chain-termination methods, de novo sequencing methods including shotgun sequencing and bridge PCR, next-generation methods including Polony sequencing, 454 pyrosequencing, Illumina sequencing, SOLID™ sequencing, Ion Torrent semiconductor sequencing, HeliScope single molecule sequencing, SMRT® sequencing, and Oxford Nanopore Technology sequencing.

Further, sequence analysis of the nucleic acid molecules can be direct or indirect. Thus, the sequence analysis can be performed on a barcoded nucleic acid molecule or it can be a molecule which is derived therefrom (e.g., a complement thereof).

Other examples of methods for sequencing include, but are not limited to, DNA hybridization methods, restriction enzyme digestion methods, Sanger sequencing methods, ligation methods, and microarray methods. Additional examples of sequencing methods that can be used include targeted sequencing, single molecule real-time sequencing, exon sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, co-amplification at lower denaturation temperature-PCR (COLD-PCR), sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, Solexa Genome Analyzer sequencing, MS-PET sequencing, whole transcriptome sequencing, and any combinations thereof.

In any of the methods described herein, it will be understood that the partitioning of the reaction mixture may partition more than one cell of the plurality of cells into more than one of a plurality of partitions. The partitioning of the reaction mixture may partition a first cell of the plurality of cells into a first partition, it may further partition a second cell of the plurality of cells into a second partition. Moreover, it may additionally partition a third cell of the plurality of cells into a third partition, a fourth cell of the plurality of cells into a fourth partition, up to hundreds of cells that are each partitioned into a separate, individual, partition. It should be understood that each and every partitioned cell need be bound to one or more in particular of the target antigen or any fragment of the target antigen. However, at least one cell of the population of cells partitioned into a partition will be bound to a target antigen and/or a fragment of target antigen.

In any of the methods described here, cell of the plurality may be enriched prior to the partitioning. The cells may be enriched for cell type, e.g., T cells, if obtained from a blood sample or may be enriched by sorting, e.g., as cells bound to the target antigen and/or one or more fragments of the target antigen.

The methods described herein can also be combined with methods described in WO 2018/075693, which is hereby incorporated by reference.

Such methods can be useful for characterizing an infusion product, e.g., with a threshold of sensitivity of 0.0249%, and may be useful for resolving heterogeneity in an infusion product.

### Further Disclosure-Partitions, Partitioning, Reagents and Processing

### Systems and Methods for Partitioning

In some aspects, such as those that have been described above, the methods provided herein include a step of partitioning, or include a step of generating barcoded nucleic acid molecules, or may include an additional processing step(s). In some aspects, the methods herein provide for a partition. This description sets forth examples, embodiments and characteristics of steps of the methods, of the partitions, and of reagents useful in the methods or as may be provided in the partitions.

In an aspect, the systems and methods described herein provide for the compartmentalization, depositing, or partitioning of one or more particles (e.g., biological particles, macromolecular constituents of biological particles, beads, reagents, etc.) into discrete compartments or partitions (referred to interchangeably herein as partitions), where each partition maintains separation of its own contents from the contents of other partitions. A partition can be a volume or subvolume wherein diffusion of contents beyond the volume or sub-volume is inhibited. For example, the partitions can include a porous matrix that is capable of entraining and/or retaining materials within its matrix.

In some embodiments disclosed herein, the partitioned particle is a labelled cell of T cell lineage, e.g. a CAR-T cell, which expresses a chimeric antigen receptor (e.g., an immune receptor) on its surface. In other examples, the partitioned particle can be a labelled cell engineered to express antigen-binding molecules (e.g., an immune receptors, antibodies or functional fragments thereof).

The term “partition,” as used herein, generally, refers to a space or volume that can be suitable to contain one or more cells, one or more species of features or compounds, or conduct one or more reactions. A partition can be a physical container, compartment, or vessel, such as a droplet, a flow cell, a reaction chamber, a reaction compartment, a tube, a well, or a microwell. In some embodiments, the compartments or partitions include partitions that are flowable within fluid streams. These partitions can include, for example, micro-vesicles that have an outer barrier surrounding an inner fluid center or core, or, in some cases, the partitions can include a porous matrix that is capable of entraining and/or retaining materials within its matrix. In some aspects, partitions comprise droplets of aqueous fluid within a non-aqueous continuous phase (e.g., oil phase). A variety of different vessels are described in, for example, U.S. Patent Application Publication No. 2014/0155295. Emulsion systems for creating stable droplets in non-aqueous or oil continuous phases are described in detail in, e.g., U.S. Patent Application Publication No. 2010/010511.

In some embodiments, a partition herein includes a space or volume that can be suitable to contain one or more species or conduct one or more reactions. A partition can be a physical compartment, such as a droplet or well. The partition can be an isolated space or volume from another space or volume. The droplet can be a first phase (e.g., aqueous phase) in a second phase (e.g., oil) immiscible with the first phase. The droplet can be a first phase in a second phase that does not phase separate from the first phase, such as, for example, a capsule or liposome in an aqueous phase. A partition can include one or more other (inner) partitions. In some cases, a partition can be a virtual compartment that can be defined and identified by an index (e.g., indexed libraries) across multiple and/or remote physical compartments. For example, a physical compartment can include a plurality of virtual compartments.

In some embodiments, the methods described herein provide for the compartmentalization, depositing or partitioning of individual cells from a sample material containing cells, into discrete partitions, where each partition maintains separation of its own contents from the contents of other partitions. Identifiers including unique identifiers (e.g., UMI) and common or universal tags, e.g., barcodes, can be previously, subsequently or concurrently delivered to the partitions that hold the compartmentalized or partitioned cells, in order to allow for the later attribution of the characteristics of the individual cells to one or more particular compartments. Further, identifiers including unique identifiers and common or universal tags, e.g., barcodes, can be coupled to labelling agents and previously, subsequently or concurrently delivered to the partitions that hold the compartmentalized or partitioned cells, in order to allow for the later attribution of the characteristics of the individual cells to one or more particular compartments. Identifiers including unique identifiers and common or universal tags, e.g., barcodes, can be delivered, for example on an oligonucleotide, to a partition via any suitable mechanism, for example by coupling the barcoded oligonucleotides to a bead. In some embodiments, the barcoded oligonucleotides are reversibly (e.g., releasably) coupled to a bead. The bead suitable for the compositions and methods of the disclosure can have different surface chemistries and/or physical volumes. In some embodiments, the bead includes a polymer gel. In some embodiments, the polymer gel is a polyacrylamide. Additional non-limiting examples of suitable beads include microparticles, nanoparticles, solid beads, magnetic beads, and microbeads. The partition can be a droplet in an emulsion. A partition can include one or more particles. A partition can include one or more types of particles. For example, a partition of the present disclosure can include one or more biological particles, e.g., labelled engineered cells, B cells, or memory B cells, and/or macromolecular constituents thereof. A partition can include one or more gel beads. A partition can include one or more cell beads. A partition can include a single gel bead, a single cell bead, or both a single cell bead and single gel bead. A partition can include one or more reagents. Alternatively, a partition can be unoccupied. For example, a partition cannot comprise a bead. Unique identifiers, such as barcodes, can be injected into the droplets previous to, subsequent to, or concurrently with droplet generation, such as via a bead, as described elsewhere herein. Microfluidic channel networks (e.g., on a chip) can be utilized to generate partitions as described herein. Alternative mechanisms can also be employed in the partitioning of individual biological particles, including porous membranes through which aqueous mixtures of cells are extruded into non-aqueous fluids.

The partitions can be flowable within fluid streams. The partitions can include, for example, micro-vesicles that have an outer barrier surrounding an inner fluid center or core. In some cases, the partitions can include a porous matrix that is capable of entraining and/or retaining materials (e.g., expressed antibodies or antigen-binding fragments thereof) within its matrix (e.g., via a capture agent configured to couple to both the matrix and the expressed antibody or antigen-binding fragment thereof). The partitions can be droplets of a first phase within a second phase, wherein the first and second phases are immiscible. For example, the partitions can be droplets of aqueous fluid within a non-aqueous continuous phase (e.g., oil phase). In another example, the partitions can be droplets of a non-aqueous fluid within an aqueous phase. In some examples, the partitions can be provided in a water-in-oil emulsion or oil-in-water emulsion. A variety of different vessels are described in, for example, U.S. Patent Application Publication No. 2014/0155295. Emulsion systems for creating stable droplets in non-aqueous or oil continuous phases are described in, for example, U.S. Patent Application Publication No. 2010/0105112.

In the case of droplets in an emulsion, allocating individual particles (e.g., labelled engineered cells) to discrete partitions can, in one non-limiting example, be accomplished by introducing a flowing stream of particles in an aqueous fluid into a flowing stream of a non-aqueous fluid, such that droplets are generated at the junction of the two streams. Fluid properties (e.g., fluid flow rates, fluid viscosities, etc.), particle properties (e.g., volume fraction, particle size, particle concentration, etc.), microfluidic architectures (e.g., channel geometry, etc.), and other parameters can be adjusted to control the occupancy of the resulting partitions (e.g., number of biological particles per partition, number of beads per partition, etc.). For example, partition occupancy can be controlled by providing the aqueous stream at a certain concentration and/or flow rate of particles. To generate single biological particle partitions, the relative flow rates of the immiscible fluids can be selected such that, on average, the partitions can contain less than one biological particle per partition in order to ensure that those partitions that are occupied are primarily singly occupied. In some cases, partitions among a plurality of partitions can contain at most one biological particle (e.g., bead, DNA, cell, such as a labelled engineered cells, T cells, or cellular material). In some embodiments, the various parameters (e.g., fluid properties, particle properties, microfluidic architectures, etc.) can be selected or adjusted such that a majority of partitions are occupied, for example, allowing for only a small percentage of unoccupied partitions. The flows and channel architectures can be controlled as to ensure a given number of singly occupied partitions, less than a certain level of unoccupied partitions and/or less than a certain level of multiply occupied partitions.

In some embodiments, the method further includes individually partitioning one or more single cells from a plurality of cells in a partition of a second plurality of partitions.

In some embodiments, at least one of the first and second plurality of partitions includes a microwell, a flow cell, a reaction chamber, a reaction compartment, or a droplet. In some embodiments, at least one of the first and second plurality of partitions includes individual droplets in emulsion. In some embodiments, the partitions of the first plurality and/or the second plurality of partition have the same reaction volume.

In the case of droplets in emulsion, allocating individual cells to discrete partitions can generally be accomplished by introducing a flowing stream of cells in an aqueous fluid into a flowing stream of a non-aqueous fluid, such that droplets are generated at the junction of the two streams. By providing the aqueous cell-containing stream at a certain concentration of cells, the occupancy of the resulting partitions (e.g., number of cells per partition) can be controlled. For example, where single cell partitions are desired, the relative flow rates of the fluids can be selected such that, on average, the partitions contain less than one cell per partition, in order to ensure that those partitions that are occupied, are primarily singly occupied. In some embodiments, the relative flow rates of the fluids can be selected such that a majority of partitions are occupied, e.g., allowing for only a small percentage of unoccupied partitions. In some embodiments, the flows and channel architectures are controlled as to ensure a desired number of singly occupied partitions, less than a certain level of unoccupied partitions and less than a certain level of multiply occupied partitions.

In some embodiments, the methods described herein can be performed such that a majority of occupied partitions include no more than one cell per occupied partition. In some embodiments, the partitioning process is performed such that fewer than 25%, fewer than 20%, fewer than 15%, fewer than 10%, fewer than 5%, fewer than 2%, or fewer than 1% the occupied partitions contain more than one cell. In some embodiments, fewer than 20% of the occupied partitions include more than one cell. In some embodiments, fewer than 10% of the occupied partitions include more than one cell per partition. In some embodiments, fewer than 5% of the occupied partitions include more than one cell per partition. In some embodiments, it is desirable to avoid the creation of excessive numbers of empty partitions. For example, from a cost perspective and/or efficiency perspective, it may be desirable to minimize the number of empty partitions. While this can be accomplished by providing sufficient numbers of cells into the partitioning zone, the Poissonian distribution can optionally be used to increase the number of partitions that include multiple cells. As such, in some embodiments described herein, the flow of one or more of the cells, or other fluids directed into the partitioning zone are performed such that no more than 50% of the generated partitions, no more than 25% of the generated partitions, or no more than 10% of the generated partitions are unoccupied. Further, in some aspects, these flows are controlled so as to present non-Poissonian distribution of single occupied partitions while providing lower levels of unoccupied partitions. Restated, in some aspects, the above noted ranges of unoccupied partitions can be achieved while still providing any of the single occupancy rates described above. For example, in some embodiments, the use of the systems and methods described herein creates resulting partitions that have multiple occupancy rates of less than 25%, less than 20%, less than 15%), less than 10%, and in some embodiments, less than 5%, while having unoccupied partitions of less than 50%), less than 40%, less than 30%, less than 20%, less than 10%, and in some embodiments, less than 5%.

Although described in terms of providing substantially singly occupied partitions, above, in some embodiments, the methods as described herein include providing multiply occupied partitions, e.g., containing two, three, four or more cells and/or beads comprising nucleic acid barcode molecules within a single partition.

In some embodiments, the reporter oligonucleotides contained within a partition are distinguishable from the reporter oligonucleotides contained within other partitions of the plurality of partitions.

In some embodiments, it may be desirable to incorporate multiple different barcode sequences within a given partition, either attached to a single or multiple beads within the partition. For example, in some cases, a mixed, but known barcode sequences set can provide greater assurance of identification in the subsequent processing, e.g., by providing a stronger address or attribution of the barcodes to a given partition, as a duplicate or independent confirmation of the output from a given partition.

### Microfluidic Channel Structures

Microfluidic channel networks (e.g., on a chip) can be utilized to generate partitions as described herein. Alternative mechanisms can also be employed in the partitioning of individual biological particles, including porous membranes through which aqueous mixtures of cells are extruded into non-aqueous fluids.

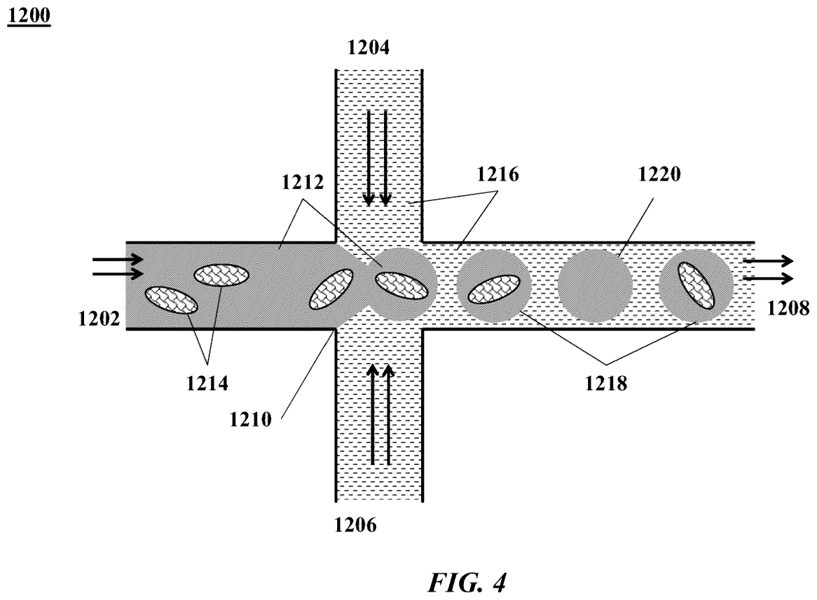

FIG. 4 shows an example of a microfluidic channel structure 1200 for partitioning individual biological particles. The channel structure 1200 can include channel segments 1202, 1204, 1206 and 1208 communicating at a channel junction 1210. In operation, a first aqueous fluid 1212 that includes suspended biological particles (e.g., cells, for example, labelled engineered chimeric antigen receptor-expressing immune cells, T cells) 1214 can be transported along channel segment 1202 into junction 1210, while a second fluid 1216 that is immiscible with the aqueous fluid 1212 is delivered to the junction 1210 from each of channel segments 1204 and 1206 to create discrete droplets 1218, 1220 of the first aqueous fluid 1212 flowing into channel segment 1208, and flowing away from junction 1210. The channel segment 1208 can be fluidically coupled to an outlet reservoir where the discrete droplets can be stored and/or harvested. A discrete droplet generated can include an individual biological particle 1214 (such as droplets 1218). A discrete droplet generated can include more than one individual biological particle (e.g., labelled T cells) 1214 (not shown in FIG. 4). A discrete droplet can contain no biological particle 1214 (such as droplet 1220). Each discrete partition can maintain separation of its own contents (e.g., individual biological particle 1214) from the contents of other partitions.

The second fluid 1216 can comprise an oil, such as a fluorinated oil, that includes a fluorosurfactant for stabilizing the resulting droplets, for example, inhibiting subsequent coalescence of the resulting droplets 1218, 1220. Examples of particularly useful partitioning fluids and fluorosurfactants are described, for example, in U.S. Patent Application Publication No. 2010/0105112.

As will be appreciated, the channel segments described herein can be coupled to any of a variety of different fluid sources or receiving components, including reservoirs, tubing, manifolds, or fluidic components of other systems. As will be appreciated, the microfluidic channel structure 1200 can have other geometries. For example, a microfluidic channel structure can have more than one channel junction. For example, a microfluidic channel structure can have 2, 3, 4, or 5 channel segments each carrying particles (e.g., biological particles, cell beads, and/or gel beads) that meet at a channel junction. Fluid can be directed to flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid can also or otherwise be controlled via applied pressure differentials, centrifugal force, electrokinetic pumping, vacuum, capillary or gravity flow, or the like.

The generated droplets can include two subsets of droplets: (1) occupied droplets 1218, containing one or more biological particles 1214, e.g., labelled engineered CAR-T cells, T cells, and (2) unoccupied droplets 1220, not containing any biological particles 1214. Occupied droplets 1218 can include singly occupied droplets (having one biological particle, such as one T cell or CAR-T cell) and multiply occupied droplets (having more than one biological particle, such as multiple T cells or CAR-T cells). As described elsewhere herein, in some cases, the majority of occupied partitions can include no more than one biological particle, e.g., labelled engineered CAR-T cells, T cells, per occupied partition and some of the generated partitions can be unoccupied (of any biological particle, or labelled engineered CAR-T cells, T cells). In some cases, though, some of the occupied partitions can include more than one biological particle, e.g., labelled engineered CAR-T cells, T cells. In some cases, the partitioning process can be controlled such that fewer than about 25% of the occupied partitions contain more than one biological particle, and in many cases, fewer than about 20% of the occupied partitions have more than one biological particle, while in some cases, fewer than about 10% or even fewer than about 5% of the occupied partitions include more than one biological particle per partition.

In some cases, it can be desirable to minimize the creation of excessive numbers of empty partitions, such as to reduce costs and/or increase efficiency. While this minimization can be achieved by providing a sufficient number of biological particles (e.g., biological particles, such as labelled engineered cells CAR-T cells, T cells, 1214) at the partitioning junction 1210, such as to ensure that at least one biological particle is encapsulated in a partition, the Poissonian distribution can expectedly increase the number of partitions that include multiple biological particles. As such, where singly occupied partitions are to be obtained, at most about 95%, 90%, 85%, 80%, 75%, 70%, 65%, 60%, 55%, 50%, 45%, 40%, 35%, 30%, 25%, 20%, 15%, 10%, 5% or less of the generated partitions can be unoccupied.

In some cases, the flow of one or more of the biological particles, such as T cells (e.g., in channel segment 1202), or other fluids directed into the partitioning junction (e.g., in channel segments 1204, 1206) can be controlled such that, in many cases, no more than about 50% of the generated partitions, no more than about 25% of the generated partitions, or no more than about 10% of the generated partitions are unoccupied. These flows can be controlled so as to present a non-Poissonian distribution of single-occupied partitions while providing lower levels of unoccupied partitions. The above noted ranges of unoccupied partitions can be achieved while still providing any of the single occupancy rates described above. For example, in many cases, the use of the systems and methods described herein can create resulting partitions that have multiple occupancy rates of less than about 25%, less than about 20%, less than about 15%, less than about 10%, and in many cases, less than about 5%, while having unoccupied partitions of less than about 50%, less than about 40%, less than about 30%, less than about 20%, less than about 10%, less than about 5%, or less.

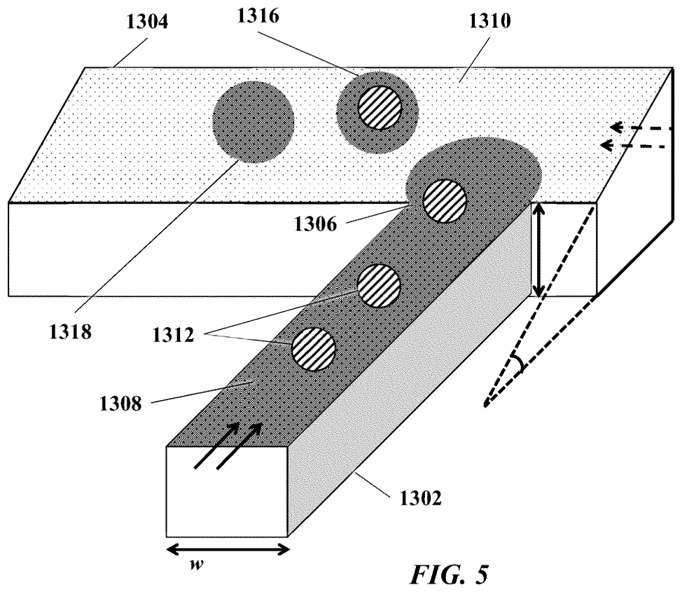

As will be appreciated, the above-described occupancy rates are also applicable to partitions that include both biological particles (e.g., cells) and additional reagents, including, but not limited to, beads (e.g., gel beads) carrying nucleic acid barcode molecules (e.g., barcoded oligonucleotides) (described in relation to FIGS. 4 and 5). The occupied partitions (e.g., at least about 10%, 20%, 30%, 40%, 50%, 60%, 70%, 80%, 90%, 95%, or 99% of the occupied partitions) can include both a bead comprising nucleic acid barcode molecules and a biological particle.

In another aspect, in addition to or as an alternative to droplet-based partitioning, biological particles (e.g., cells) may be encapsulated within a particulate material to form a “cell bead”.

The cell bead can include other reagents. Encapsulation of biological particles, e.g., labelled engineered cells, can be performed by a variety of processes. Such processes can combine an aqueous fluid containing the biological particles with a polymeric precursor material that can be capable of being formed into a gel or other solid or semi-solid matrix upon application of a particular stimulus to the polymer precursor. Such stimuli can include, for example, thermal stimuli (e.g., cither heating or cooling), photo-stimuli (e.g., through photo-curing), chemical stimuli (e.g., through crosslinking, polymerization initiation of the precursor (e.g., through added initiators)), mechanical stimuli, or a combination thereof.

Encapsulation of biological particles, e.g., labelled engineered CAR-T cells, T cells, can be performed by a variety of methods. For example, air knife droplet or aerosol generators may be used to dispense droplets of precursor fluids into gelling solutions in order to form cell beads that include individual biological particles or small groups of biological particles. Likewise, membrane-based encapsulation systems may be used to generate cell beads comprising encapsulated biological particles as described herein. Microfluidic systems of the present disclosure, such as that shown in FIG. 4, may be readily used in encapsulating biological particles (e.g., cells) as described herein. Exemplary methods for encapsulating biological particles (e.g., cells) are also further described in U.S. Patent Application Pub. No. US 2015/0376609 and PCT/US2018/016019. In particular, and with reference to FIG. 4, the aqueous fluid 1212 comprising (i) the biological particles 1214 and (ii) the polymer precursor material (not shown) is flowed into channel junction 1210, where it is partitioned into droplets 1218, 1220 through the flow of non-aqueous fluid 1216. In the case of encapsulation methods, non-aqueous fluid 1216 may also include an initiator (not shown) to cause polymerization and/or crosslinking of the polymer precursor to form the microcapsule that includes the entrained biological particles. Examples of polymer precursor/initiator pairs include those described in U.S. Patent Application Publication No. 2014/0378345.

For example, in the case where the polymer precursor material comprises a linear polymer material, such as a linear polyacrylamide, PEG, or other linear polymeric material, the activation agent can include a cross-linking agent, or a chemical that activates a cross-linking agent within the formed droplets. Likewise, for polymer precursors that comprise polymerizable monomers, the activation agent can include a polymerization initiator. For example, in certain cases, where the polymer precursor comprises a mixture of acrylamide monomer with a N,N′-bis-(acryloyl) cystamine (BAC) comonomer, an agent such as tetraethylmethylenediamine (TEMED) can be provided within the second fluid streams 1216 in channel segments 1204 and 1206, which can initiate the copolymerization of the acrylamide and BAC into a cross-linked polymer network, or hydrogel.

Upon contact of the second fluid stream 1216 with the first fluid stream 1212 at junction 1210, during formation of droplets, the TEMED can diffuse from the second fluid 1216 into the aqueous fluid 1212 comprising the linear polyacrylamide, which will activate the crosslinking of the polyacrylamide within the droplets 1218, 1220, resulting in the formation of gel (e.g., hydrogel) cell beads, as solid or semi-solid beads or particles entraining the cells (e.g., T cells) 1214. Although described in terms of polyacrylamide encapsulation, other “activatable” encapsulation compositions can also be employed in the context of the methods and compositions described herein. For example, formation of alginate droplets followed by exposure to divalent metal ions (e.g., Ca²⁺ ions), can be used as an encapsulation process using the described processes. Likewise, agarose droplets can also be transformed into capsules through temperature based gelling (e.g., upon cooling, etc.).

In some cases, encapsulated biological particles can be selectively releasable from the cell bead, such as through passage of time or upon application of a particular stimulus, that degrades the encapsulating material sufficiently to allow the biological particles (e.g., labelled T cells), or its other contents to be released from the encapsulating material, such as into a partition (e.g., droplet). For example, in the case of the polyacrylamide polymer described above, degradation of the polymer can be accomplished through the introduction of an appropriate reducing agent, such as DTT or the like, to cleave disulfide bonds that cross-link the polymer matrix. See, for example, U.S. Patent Application Publication No. 2014/0378345.

The biological particle (e.g., labelled T cells), can be subjected to other conditions sufficient to polymerize or gel the precursors. The conditions sufficient to polymerize or gel the precursors can include exposure to heating, cooling, electromagnetic radiation, and/or light. The conditions sufficient to polymerize or gel the precursors can include any conditions sufficient to polymerize or gel the precursors. Following polymerization or gelling, a polymer or gel can be formed around the biological particle (e.g., labelled T cells). The polymer or gel can be diffusively permeable to chemical or biochemical reagents. The polymer or gel can be diffusively impermeable to macromolecular constituents (e.g., secreted antibodies or antigen-binding fragments thereof) of the biological particle (e.g., labelled T cells). In this manner, the polymer or gel can act to allow the biological particle (e.g., labelled T cells) to be subjected to chemical or biochemical operations while spatially confining the macromolecular constituents to a region of the droplet defined by the polymer or gel. The polymer or gel can include one or more of disulfide cross-linked polyacrylamide, agarose, alginate, polyvinyl alcohol, polyethylene glycol (PEG)-diacrylate, PEG-acrylate, PEG-thiol, PEG-azide, PEG-alkyne, other acrylates, chitosan, hyaluronic acid, collagen, fibrin, gelatin, or elastin. The polymer or gel can include any other polymer or gel.

The polymer or gel can be functionalized (e.g., coupled to a capture agent) to bind to targeted analytes (e.g., secreted antibodies or antigen-binding fragment thereof), such as nucleic acids, proteins, carbohydrates, lipids or other analytes. The polymer or gel can be polymerized or gelled via a passive mechanism. The polymer or gel can be stable in alkaline conditions or at elevated temperature. The polymer or gel can have mechanical properties similar to the mechanical properties of the bead. For instance, the polymer or gel can be of a similar size to the bead. The polymer or gel can have a mechanical strength (e.g., tensile strength) similar to that of the bead. The polymer or gel can be of a lower density than an oil. The polymer or gel can be of a density that is roughly similar to that of a buffer. The polymer or gel can have a tunable pore size. The pore size can be chosen to, for instance, retain denatured nucleic acids. The pore size can be chosen to maintain diffusive permeability to exogenous chemicals such as sodium hydroxide (NaOH) and/or endogenous chemicals such as inhibitors. The polymer or gel can be biocompatible. The polymer or gel can maintain or enhance cell viability. The polymer or gel can be biochemically compatible. The polymer or gel can be polymerized and/or depolymerized thermally, chemically, enzymatically, and/or optically.

The polymer can include poly(acrylamide-co-acrylic acid) crosslinked with disulfide linkages. The preparation of the polymer can include a two-step reaction. In the first activation step, poly(acrylamide-co-acrylic acid) can be exposed to an acylating agent to convert carboxylic acids to esters. For instance, the poly(acrylamide-co-acrylic acid) can be exposed to 4-(4,6-dimethoxy-1,3,5-triazin-2-yl)-4-methylmorpholinium chloride (DMTMM). The polyacrylamide-co-acrylic acid can be exposed to other salts of 4-(4,6-dimethoxy-1,3,5-triazin-2-yl)-4-methylmorpholinium. In the second cross-linking step, the ester formed in the first step can be exposed to a disulfide crosslinking agent. For instance, the ester can be exposed to cystamine (2,2′-dithiobis(ethylamine)). Following the two steps, the biological particle can be surrounded by polyacrylamide strands linked together by disulfide bridges. In this manner, the biological particle can be encased inside of or comprise a gel or matrix (e.g., polymer matrix) to form a “cell bead.” A cell bead can contain biological particles (e.g., labelled B cells) or macromolecular constituents (e.g., RNA, DNA, proteins, secreted antibodies or antigen-binding fragments thereof etc.) of biological particles. A cell bead can include a single cell or multiple cells, or a derivative of the single cell or multiple cells. For example after lysing and washing the cells, inhibitory components from cell lysates can be washed away and the macromolecular constituents can be bound as cell beads. Systems and methods disclosed herein can be applicable to both (i) cell beads (and/or droplets or other partitions) containing biological particles and (ii) cell beads (and/or droplets or other partitions) containing macromolecular constituents of biological particles.

Encapsulated biological particles (e.g., labelled T cells) can provide certain potential advantages of being more storable and more portable than droplet-based partitioned biological particles. Furthermore, in some cases, it can be desirable to allow biological particles (e.g., labelled T cells) to incubate for a select period of time before analysis, such as in order to characterize changes in such biological particles over time, either in the presence or absence of different stimuli (e.g., cytokines, antigens, etc.). In such cases, encapsulation can allow for longer incubation than partitioning in emulsion droplets, although in some cases, droplet partitioned biological particles can also be incubated for different periods of time, e.g., at least 10 seconds, at least 30 seconds, at least 1 minute, at least 5 minutes, at least 10 minutes, at least 30 minutes, at least 1 hour, at least 2 hours, at least 5 hours, or at least 10 hours or more. The encapsulation of biological particles (e.g., labelled T cells) can constitute the partitioning of the biological particles into which other reagents are co-partitioned. Alternatively or in addition, encapsulated biological particles can be readily deposited into other partitions (e.g., droplets) as described above.

### Microwells

As described herein, one or more processes can be performed in a partition, which can be a well. The well can be a well of a plurality of wells of a substrate, such as a microwell of a microwell array or plate, or the well can be a microwell or microchamber of a device (e.g., microfluidic device) comprising a substrate. The well can be a well of a well array or plate, or the well can be a well or chamber of a device (e.g., fluidic device). Accordingly, the wells or microwells can assume an “open” configuration, in which the wells or microwells are exposed to the environment (e.g., contain an open surface) and are accessible on one planar face of the substrate, or the wells or microwells can assume a “closed” or “sealed” configuration, in which the microwells are not accessible on a planar face of the substrate. In some instances, the wells or microwells can be configured to toggle between “open” and “closed” configurations. For instance, an “open” microwell or set of microwells can be “closed” or “sealed” using a membrane (e.g., semi-permeable membrane), an oil (e.g., fluorinated oil to cover an aqueous solution), or a lid, as described elsewhere herein. The wells or microwells can be initially provided in a “closed” or “sealed” configuration, wherein they are not accessible on a planar surface of the substrate without an external force. For instance, the “closed” or “sealed” configuration can include a substrate such as a sealing film or foil that is puncturable or pierceable by pipette tip(s). Suitable materials for the substrate include, without limitation, polyester, polypropylene, polyethylene, vinyl, and aluminum foil.

In some embodiments, the well can have a volume of less than 1 milliliter (mL). For example, the well can be configured to hold a volume of at most 1000 microliters (μL), at most 100 μL, at most 10 μL, at most 1 μL, at most 100 nanoliters (nL), at most 10 nL, at most 1 nL, at most 100 picoliters (pL), at most 10 (pL), or less. The well can be configured to hold a volume of about 1000 μL, about 100 μL, about 10 μL, about 1 μL, about 100 nL, about 10 nL, about 1 nL, about 100 pL, about 10 pL, etc. The well can be configured to hold a volume of at least 10 pL, at least 100 pL, at least 1 nL, at least 10 nL, at least 100 nL, at least 1 μL, at least 10 μL, at least 100 μL, at least 1000 μL, or more. The well can be configured to hold a volume in a range of volumes listed herein, for example, from about 5 nL to about 20 nL, from about 1 nL to about 100 nL, from about 500 pL to about 100 μL, etc. The well can be of a plurality of wells that have varying volumes and can be configured to hold a volume appropriate to accommodate any of the partition volumes described herein.

In some instances, a microwell array or plate includes a single variety of microwells. In some instances, a microwell array or plate includes a variety of microwells. For instance, the microwell array or plate can include one or more types of microwells within a single microwell array or plate. The types of microwells can have different dimensions (e.g., length, width, diameter, depth, cross-sectional area, etc.), shapes (e.g., circular, triangular, square, rectangular, pentagonal, hexagonal, heptagonal, octagonal, nonagonal, decagonal, etc.), aspect ratios, or other physical characteristics. The microwell array or plate can include any number of different types of microwells. For example, the microwell array or plate can include 1, 2, 3, 4, 5, 6, 7, 8, 9, 10, 20, 30, 40, 50, 60, 70, 80, 90, 100, 200, 300, 400, 500, 600, 700, 800, 900, 1000 or more different types of microwells. A well can have any dimension (e.g., length, width, diameter, depth, cross-sectional area, volume, etc.), shape (e.g., circular, triangular, square, rectangular, pentagonal, hexagonal, heptagonal, octagonal, nonagonal, decagonal, other polygonal, etc.), aspect ratios, or other physical characteristics described herein with respect to any well.

In certain instances, the microwell array or plate includes different types of microwells that are located adjacent to one another within the array or plate. For example, a microwell with one set of dimensions can be located adjacent to and in contact with another microwell with a different set of dimensions. Similarly, microwells of different geometries can be placed adjacent to or in contact with one another. The adjacent microwells can be configured to hold different articles; for example, one microwell can be used to contain a cell, cell bead, or other sample (e.g., cellular components, nucleic acid molecules, etc.) while the adjacent microwell can be used to contain a droplet, bead, or other reagent. In some cases, the adjacent microwells can be configured to merge the contents held within, e.g., upon application of a stimulus, or spontaneously, upon contact of the articles in each microwell.

As is described elsewhere herein, a plurality of partitions can be used in the systems, compositions, and methods described herein. For example, any suitable number of partitions (e.g., wells or droplets) can be generated or otherwise provided. For example, in the case when wells are used, at least about 1,000 wells, at least about 5,000 wells, at least about 10,000 wells, at least about 50,000 wells, at least about 100,000 wells, at least about 500,000 wells, at least about 1,000,000 wells, at least about 5,000,000 wells at least about 10,000,000 wells, at least about 50,000,000 wells, at least about 100,000,000 wells, at least about 500,000,000 wells, at least about 1,000,000,000 wells, or more wells can be generated or otherwise provided. Moreover, the plurality of wells can include both unoccupied wells (e.g., empty wells) and occupied wells.

A well can include any of the reagents described herein, or combinations thereof. These reagents can include, for example, barcode molecules, enzymes, adapters, and combinations thereof. The reagents can be physically separated from a sample (for example, a cell, cell bead, or cellular components, e.g., proteins, nucleic acid molecules, etc.) that is placed in the well. This physical separation can be accomplished by containing the reagents within, or coupling to, a bead that is placed within a well. The physical separation can also be accomplished by dispensing the reagents in the well and overlaying the reagents with a layer that is, for example, dissolvable, meltable, or permeable prior to introducing the polynucleotide sample into the well. This layer can be, for example, an oil, wax, membrane (e.g., semi-permeable membrane), or the like. The well can be sealed at any point, for example, after addition of the bead, after addition of the reagents, or after addition of either of these components. The sealing of the well can be useful for a variety of purposes, including preventing escape of beads or loaded reagents from the well, permitting select delivery of certain reagents (e.g., via the use of a semi-permeable membrane), for storage of the well prior to or following further processing, etc.

Once sealed, the well may be subjected to conditions for further processing of a cell (or cells) in the well. For instance, reagents in the well may allow further processing of the cell, e.g., cell lysis, as further described herein. Alternatively, the well (or wells such as those of a well-based array) comprising the cell (or cells) may be subjected to freeze-thaw cycling to process the cell (or cells), e.g., cell lysis. The well containing the cell may be subjected to freezing temperatures (e.g., 0° C., below 0° C., −5° C., −10° C., −15° C., −20° C., −25° C., −30° C., −35° C., −40° C., −45° C., −50° C., −55° C., −60° C., −65° C., −70° C., −80° C., or −85° C.). Freezing may be performed in a suitable manner, e.g., sub-zero freezer or a dry ice/ethanol bath. Following an initial freezing, the well (or wells) comprising the cell (or cells) may be subjected to freeze-thaw cycles to lyse the cell (or cells). In one embodiment, the initially frozen well (or wells) are thawed to a temperature above freezing (e.g., 4° C. or above, 8° C. or above, 12° C. or above, 16° C. or above, 20° C. or above, room temperature, or 25° C. or above). In another embodiment, the freezing is performed for less than 10 minutes (e.g., 5 minutes or 7 minutes) followed by thawing at room temperature for less than 10 minutes (e.g., 5 minutes or 7 minutes). This freeze-thaw cycle may be repeated a number of times, e.g., 2, 3, 4 or more times, to obtain lysis of the cell (or cells) in the well (or wells). In one embodiment, the freezing, thawing and/or freeze/thaw cycling is performed in the absence of a lysis buffer. Additional disclosure related to freeze-thaw cycling is provided in WO2019165181A1, which is incorporated herein by reference in its entirety

A well can include free reagents and/or reagents encapsulated in, or otherwise coupled to or associated with, beads or droplets. In some embodiments, any of the reagents described in this disclosure can be encapsulated in, or otherwise coupled to, a droplet or bead, with any chemicals, particles, and elements suitable for sample processing reactions involving biomolecules, such as, but not limited to, nucleic acid molecules and proteins. For example, a bead or droplet used in a sample preparation reaction for DNA sequencing can include one or more of the following reagents: enzymes, restriction enzymes (e.g., multiple cutters), ligase, polymerase, fluorophores, oligonucleotide barcodes, adapters, buffers, nucleotides (e.g., dNTPs, ddNTPs) and the like.

Additional examples of reagents include, but are not limited to: buffers, acidic solution, basic solution, temperature-sensitive enzymes, pH-sensitive enzymes, light-sensitive enzymes, metals, metal ions, magnesium chloride, sodium chloride, manganese, aqueous buffer, mild buffer, ionic buffer, inhibitor, enzyme, protein, polynucleotide, antibodies, saccharides, lipid, oil, salt, ion, detergents, ionic detergents, non-ionic detergents, oligonucleotides, nucleotides, deoxyribonucleotide triphosphates (dNTPs), dideoxyribonucleotide triphosphates (ddNTPs), DNA, RNA, peptide polynucleotides, complementary DNA (cDNA), double stranded DNA (dsDNA), single stranded DNA (ssDNA), plasmid DNA, cosmid DNA, chromosomal DNA, genomic DNA, viral DNA, bacterial DNA, mtDNA (mitochondrial DNA), mRNA, rRNA, IRNA, nRNA, siRNA, snRNA, snoRNA, scaRNA, microRNA, dsRNA, ribozyme, riboswitch and viral RNA, polymerase, ligase, restriction enzymes, proteases, nucleases, protease inhibitors, nuclease inhibitors, chelating agents, reducing agents, oxidizing agents, fluorophores, probes, chromophores, dyes, organics, emulsifiers, surfactants, stabilizers, polymers, water, small molecules, pharmaceuticals, radioactive molecules, preservatives, antibiotics, aptamers, and pharmaceutical drug compounds. As described herein, one or more reagents in the well can be used to perform one or more reactions, including but not limited to: cell lysis, cell fixation, permeabilization, nucleic acid reactions, e.g., nucleic acid extension reactions, amplification, reverse transcription, transposase reactions (e.g., tagmentation), etc.

The wells disclosed herein can be provided as a part of a kit. For example, a kit can include instructions for use, a microwell array or device, and reagents (e.g., beads). The kit can include any useful reagents for performing the processes described herein, e.g., nucleic acid reactions, barcoding of nucleic acid molecules, sample processing (e.g., for cell lysis, fixation, and/or permeabilization).

In some cases, a well includes a bead or droplet that includes a set of reagents that has a similar attribute, for example, a set of enzymes, a set of minerals, a set of oligonucleotides, a mixture of different barcode molecules, a mixture of identical barcode molecules. In other cases, a bead or droplet includes a heterogeneous mixture of reagents. In some cases, the heterogeneous mixture of reagents can include all components necessary to perform a reaction. In some cases, such mixture can include all components necessary to perform a reaction, except for 1, 2, 3, 4, 5, or more components necessary to perform a reaction. In some cases, such additional components are contained within, or otherwise coupled to, a different droplet or bead, or within a solution within a partition (e.g., microwell) of the system.

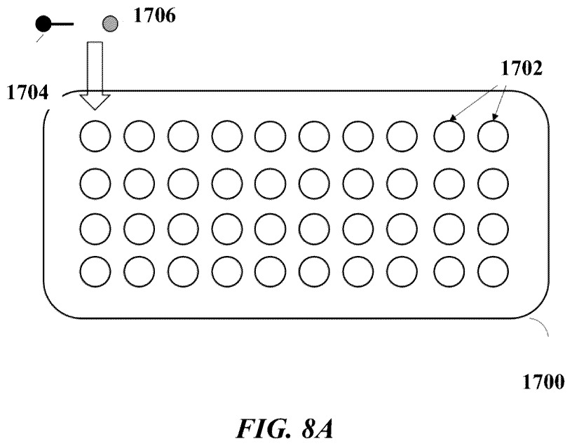

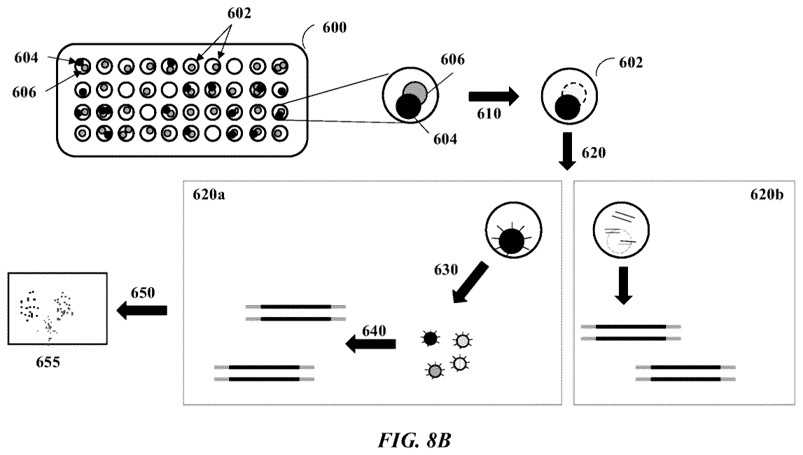

A non-limiting example of a microwell array in accordance with some embodiments of the disclosure is schematically presented in FIG. 8. In this example, the array can be contained within a substrate 1700. The substrate 1700 includes a plurality of wells 1702. The wells 1702 can be of any size or shape, and the spacing between the wells, the number of wells per substrate, as well as the density of the wells on the substrate 1700 can be modified, depending on the particular application. In one such example application, a sample molecule 1706, which can include a cell or cellular components (e.g., nucleic acid molecules) is co-partitioned with a bead 1704, which can include a nucleic acid barcode molecule coupled thereto. The wells 1702 can be loaded using gravity or other loading technique (e.g., centrifugation, liquid handler, acoustic loading, optoelectronic, etc.). In some instances, at least one of the wells 1702 contains a single sample molecule 1706 (e.g., cell) and a single bead 1704.

Reagents can be loaded into a well either sequentially or concurrently. In some cases, reagents are introduced to the device either before or after a particular operation. In some cases, reagents (which can be provided, in certain instances, in droplets or beads) are introduced sequentially such that different reactions or operations occur at different steps. The reagents (or droplets, or beads) can also be loaded at operations interspersed with a reaction or operation step. For example, or droplets or beads including reagents for fragmenting polynucleotides (e.g., restriction enzymes) and/or other enzymes (e.g., transposases, ligases, polymerases, etc.) can be loaded into the well or plurality of wells, followed by loading of droplets or beads including reagents for attaching nucleic acid barcode molecules to a sample nucleic acid molecule. Reagents can be provided concurrently or sequentially with a sample, e.g., a cell or cellular components (e.g., organelles, proteins, nucleic acid molecules, carbohydrates, lipids, etc.). Accordingly, use of wells can be useful in performing multi-step operations or reactions.

As described elsewhere herein, the nucleic acid barcode molecules and other reagents can be contained within a bead or droplet. These beads or droplets can be loaded into a partition (e.g., a microwell) before, after, or concurrently with the loading of a cell, such that each cell is contacted with a different bead or droplet. This technique can be used to attach a unique nucleic acid barcode molecule to nucleic acid molecules obtained from each cell. Alternatively or in addition, the sample nucleic acid molecules can be attached to a support. For example, the partition (e.g., microwell) can include a bead which has coupled thereto a plurality of nucleic acid barcode molecules. The sample nucleic acid molecules, or derivatives thereof, can couple or attach to the nucleic acid barcode molecules attached on the support. The resulting barcoded nucleic acid molecules can then be removed from the partition, and in some instances, pooled and sequenced. In such cases, the nucleic acid barcode sequences can be used to trace the origin of the sample nucleic acid molecule. For example, polynucleotides with identical barcodes can be determined to originate from the same cell or partition, while polynucleotides with different barcodes can be determined to originate from different cells or partitions.

The samples or reagents can be loaded in the wells or microwells using a variety of approaches. For example, the samples (e.g., a cell, cell bead, or cellular component) or reagents (as described herein) can be loaded into the well or microwell using an external force, e.g., gravitational force, electrical force, magnetic force, or using mechanisms to drive the sample or reagents into the well, for example, via pressure-driven flow, centrifugation, optoelectronics, acoustic loading, electrokinetic pumping, vacuum, capillary flow, etc. In certain cases, a fluid handling system can be used to load the samples or reagents into the well. The loading of the samples or reagents can follow a Poissonian distribution or a non-Poissonian distribution, e.g., super Poisson or sub-Poisson. The geometry, spacing between wells, density, and size of the microwells can be modified to accommodate a useful sample or reagent distribution; for example, the size and spacing of the microwells can be adjusted such that the sample or reagents can be distributed in a super-Poissonian fashion.

In one non-limiting example, the microwell array or plate includes pairs of microwells, in which each pair of microwells is configured to hold a droplet (e.g., including a single cell) and a single bead (such as those described herein, which can, in some instances, also be encapsulated in a droplet). The droplet and the bead (or droplet containing the bead) can be loaded simultaneously or sequentially, and the droplet and the bead can be merged, e.g., upon contact of the droplet and the bead, or upon application of a stimulus (e.g., external force, agitation, heat, light, magnetic or electric force, etc.). In some cases, the loading of the droplet and the bead is super-Poissonian. In other examples of pairs of microwells, the wells are configured to hold two droplets including different reagents and/or samples, which are merged upon contact or upon application of a stimulus. In such instances, the droplet of one microwell of the pair can include reagents that can react with an agent in the droplet of the other microwell of the pair. For example, one droplet can include reagents that are configured to release the nucleic acid barcode molecules of a bead contained in another droplet, located in the adjacent microwell. Upon merging of the droplets, the nucleic acid barcode molecules can be released from the bead into the partition (e.g., the microwell or microwell pair that are in contact), and further processing can be performed (e.g., barcoding, nucleic acid reactions, etc.). In cases where intact or live cells are loaded in the microwells, one of the droplets can include lysis reagents for lysing the cell upon droplet merging.

In some embodiments, a droplet or bead can be partitioned into a well. The droplets can be selected or subjected to pre-processing prior to loading into a well. For instance, the droplets can include cells, and only certain droplets, such as those containing a single cell (or at least one cell), can be selected for use in loading of the wells. Such a pre-selection process can be useful in efficient loading of single cells, such as to obtain a non-Poissonian distribution, or to pre-filter cells for a selected characteristic prior to further partitioning in the wells. Additionally, the technique can be useful in obtaining or preventing cell doublet or multiplet formation prior to or during loading of the microwell.

In some embodiments, the wells can include nucleic acid barcode molecules attached thereto. The nucleic acid barcode molecules can be attached to a surface of the well (e.g., a wall of the well). The nucleic acid barcode molecules may be attached to a droplet or bead that has been partitioned into the well. The nucleic acid barcode molecule (e.g., a partition barcode sequence) of one well can differ from the nucleic acid barcode molecule of another well, which can permit identification of the contents contained with a single partition or well. In some embodiments, the nucleic acid barcode molecule can include a spatial barcode sequence that can identify a spatial coordinate of a well, such as within the well array or well plate. In some embodiments, the nucleic acid barcode molecule can include a unique molecular identifier for individual molecule identification. In some instances, the nucleic acid barcode molecules can be configured to attach to or capture a nucleic acid molecule within a sample or cell distributed in the well. For example, the nucleic acid barcode molecules can include a capture sequence that can be used to capture or hybridize to a nucleic acid molecule (e.g., RNA, DNA) within the sample. In some embodiments, the nucleic acid barcode molecules can be releasable from the microwell. In some instances, the nucleic acid barcode molecules may be releasable from the bead or droplet. For example, the nucleic acid barcode molecules can include a chemical cross-linker which can be cleaved upon application of a stimulus (e.g., photo-, magnetic, chemical, biological, stimulus). The released nucleic acid barcode molecules, which can be hybridized or configured to hybridize to a sample nucleic acid molecule, can be collected and pooled for further processing, which can include nucleic acid processing (e.g., amplification, extension, reverse transcription, etc.) and/or characterization (e.g., sequencing). In some instances nucleic acid barcode molecules attached to a bead or droplet in a well may be hybridized to sample nucleic acid molecules, and the bead with the sample nucleic acid molecules hybridized thereto may be collected and pooled for further processing, which can include nucleic acid processing (e.g., amplification, extension, reverse transcription, etc.) and/or characterization (e.g., sequencing). In such cases, the unique partition barcode sequences can be used to identify the cell or partition from which a nucleic acid molecule originated.

Characterization of samples within a well can be performed. Such characterization can include, in non-limiting examples, imaging of the sample (e.g., cell, cell bead, or cellular components) or derivatives thereof. Characterization techniques such as microscopy or imaging can be useful in measuring sample profiles in fixed spatial locations. For example, when cells are partitioned, optionally with beads, imaging of each microwell and the contents contained therein can provide useful information on cell doublet formation (e.g., frequency, spatial locations, etc.), cell-bead pair efficiency, cell viability, cell size, cell morphology, expression level of a biomarker (e.g., a surface marker, a fluorescently labeled molecule therein, etc.), cell or bead loading rate, number of cell-bead pairs, etc. In some instances, imaging can be used to characterize live cells in the wells, including, but not limited to: dynamic live-cell tracking, cell-cell interactions (when two or more cells are co-partitioned), cell proliferation, etc. Alternatively or in addition to, imaging can be used to characterize a quantity of amplification products in the well.

In operation, a well can be loaded with a sample and reagents, simultaneously or sequentially. When cells or cell beads are loaded, the well can be subjected to washing, e.g., to remove excess cells from the well, microwell array, or plate. Similarly, washing can be performed to remove excess beads or other reagents from the well, microwell array, or plate. In the instances where live cells are used, the cells can be lysed in the individual partitions to release the intracellular components or cellular analytes. Alternatively, the cells can be fixed or permeabilized in the individual partitions. The intracellular components or cellular analytes can couple to a support, e.g., on a surface of the microwell, on a solid support (e.g., bead), or they can be collected for further downstream processing. For example, after cell lysis, the intracellular components or cellular analytes can be transferred to individual droplets or other partitions for barcoding. Alternatively, or in addition, the intracellular components or cellular analytes (e.g., nucleic acid molecules) can couple to a bead including a nucleic acid barcode molecule; subsequently, the bead can be collected and further processed, e.g., subjected to nucleic acid reaction such as reverse transcription, amplification, or extension, and the nucleic acid molecules thereon can be further characterized, e.g., via sequencing. Alternatively, or in addition, the intracellular components or cellular analytes can be barcoded in the well (e.g., using a bead including nucleic acid barcode molecules that are releasable or on a surface of the microwell including nucleic acid barcode molecules). The barcoded nucleic acid molecules or analytes can be further processed in the well, or the barcoded nucleic acid molecules or analytes can be collected from the individual partitions and subjected to further processing outside the partition. Further processing can include nucleic acid processing (e.g., performing an amplification, extension) or characterization (e.g., fluorescence monitoring of amplified molecules, sequencing). At any suitable or useful step, the well (or microwell array or plate) can be sealed (e.g., using an oil, membrane, wax, etc.), which enables storage of the assay or selective introduction of additional reagents.

Once sealed, the well may be subjected to conditions for further processing of a biological particle (e.g., a cell, a cell bead, or a nucleus) in the well. For instance, reagents in the well may allow further processing of the biological particle, e.g., lysis of the cell or nucleus, as further described herein. Alternatively, the well (or wells such as those of a well-based array) comprising the biological particle (e.g., cell, cell bead, or nucleus) may be subjected to freeze-thaw cycling to process the biological particle(s), e.g., lysis of a cell or nucleus. The well containing the biological particle (e.g., cell, cell bead, or nucleus) may be subjected to freezing temperatures (e.g., 0° C., below 0° C., −5° C., −10° C., −15° C., −20° C., −25° C., −30° C., −35° C., −40° C., −45° C., −50° C., −55° C., −60° C., −65° C., −70° C., −80° C., or −85° C.). Freezing may be performed in a suitable manner, e.g., sub-zero freezer or a dry ice/ethanol bath. Following an initial freezing, the well (or wells) comprising the biological particle(s) (e.g., cell(s), cell bead(s), nucleus or nuclei) may be subjected to freeze thaw cycles to lyse biological particle(s). In one embodiment, the initially frozen well (or wells) are thawed to a temperature above freezing (e.g., room temperature or 25° C.). In another embodiment, the freezing is performed for less than 10 minutes (e.g., 5 minutes or 7 minutes) followed by thawing at room temperature for less than 10 minutes (e.g., 5 minutes or 7 minutes). This freeze-thaw cycle may be repeated a number of times, e.g., 2, 3, or 4 times, to obtain lysis of the biological particle(s) (e.g., cell(s), cell bead(s), nucleus, or nuclei) in the well (or wells). In one embodiment, the freezing, thawing and/or freeze/thaw cycling is performed in the absence of a lysis buffer. Additional disclosure related to freeze-thaw cycling is provided in WO2019165181A1, which is incorporated herein by reference in its entirety.

FIG. 8B schematically shows an example workflow for processing nucleic acid molecules within a sample. A substrate 600 comprising a plurality of microwells 602 may be provided. A sample 606 which may comprise a cell, cell bead, cellular components or analytes (e.g., proteins and/or nucleic acid molecules) can be co-partitioned, in a plurality of microwells 602, with a plurality of beads 604 comprising nucleic acid barcode molecules. During process 610, the sample 606 may be processed within the partition. For instance, in the case of live cells, the cell may be subjected to conditions sufficient to lyse the cells and release the analytes contained therein. In process 620, the bead 604 may be further processed. By way of example, processes 620a and 620b schematically illustrate different workflows, depending on the properties of the bead 604.

In 620a, the bead comprises nucleic acid barcode molecules that are attached thereto, and sample nucleic acid molecules (e.g., RNA, DNA) may attach, e.g., via hybridization or ligation, to the nucleic acid barcode molecules. Such attachment may occur on the bead. Such beads may be solid beads or magnetic beads to allow for removal from the microwell array. In process 630, the beads 604 from multiple wells 602 may be collected and pooled. Further processing may be performed in process 640. For example, one or more nucleic acid reactions may be performed, such as reverse transcription, nucleic acid extension, amplification, ligation, transposition, etc. In some embodiments, one or more reactions occur on the bead using the sample nucleic acid molecules captured on the bead via the nucleic acid barcode molecules on the bead, e.g., sample nucleic acid molecules hybridized to complementary sequences of the nucleic acid barcode molecules on the bead. In some instances, adapter sequences are ligated to the nucleic acid molecules, or derivatives thereof, as described elsewhere herein. For instance, sequencing primer sequences may be appended to each end of the nucleic acid molecule. In process 650, further characterization, such as sequencing may be performed to generate sequencing reads. The sequencing reads may yield information on individual cells or populations of cells, which may be represented visually or graphically, e.g., in a plot 655.

In 620b, the bead comprises nucleic acid barcode molecules that are releasably attached thereto, as described below. The bead may degrade or otherwise release the nucleic acid barcode molecules into the well 602; the nucleic acid barcode molecules may then be used to barcode nucleic acid molecules within the well 602. Further processing may be performed either inside the partition or outside the partition. For example, one or more nucleic acid reactions may be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences may be appended to each end of the nucleic acid molecule. In process 650, further characterization, such as sequencing may be performed to generate sequencing reads. The sequencing reads may yield information on individual cells or populations of cells, which may be represented visually or graphically, e.g., in a plot 655.

In 620b, the bead comprises nucleic acid barcode molecules that are releasably attached thereto, as described below. The bead may degrade or otherwise release the nucleic acid barcode molecules into the well 602; the nucleic acid barcode molecules may then be used to barcode nucleic acid molecules within the well 602. Further processing may be performed either inside the partition or outside the partition. For example, one or more nucleic acid reactions may be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences may be appended to each end of the nucleic acid molecule. In process 650, further characterization, such as sequencing may be performed to generate sequencing reads. The sequencing reads may yield information on individual cells or populations of cells, which may be represented visually or graphically, e.g., in a plot 655.

### Beads

In some embodiments of the disclosure, a partition can include one or more unique identifiers, such as barcodes (e.g., a plurality of nucleic acid barcode molecules which can be, for example, a plurality of partition barcode sequences). Barcodes can be previously, subsequently or concurrently delivered to the partitions that hold the compartmentalized or partitioned biological particle (e.g., labelled B cells). For example, barcodes can be injected into droplets previous to, subsequent to, or concurrently with droplet generation. In some embodiments, the delivery of the barcodes to a particular partition allows for the later attribution of the characteristics of the individual biological particle (e.g., labelled B cells) to the particular partition. Barcodes can be delivered, for example on a nucleic acid molecule (e.g., a barcoded oligonucleotide), to a partition via any suitable mechanism. In some embodiments, nucleic acid barcode molecules can be delivered to a partition via a bead. Beads are described in further detail below.

In some embodiments, nucleic acid barcode molecules can be initially associated with the bead and then released from the bead. In some embodiments, release of the nucleic acid barcode molecules can be passive (e.g., by diffusion out of the bead). In addition or alternatively, release from the bead can be upon application of a stimulus which allows the barcoded nucleic acid nucleic acid molecules to dissociate or to be released from the bead. Such stimulus can disrupt the bead, an interaction that couples the nucleic acid barcode molecules to or within the bead, or both. Such stimulus can include, for example, a thermal stimulus, photo-stimulus, chemical stimulus (e.g., change in pH or use of a reducing agent), a mechanical stimulus, a radiation stimulus; a biological stimulus (e.g., enzyme), or any combination thereof. Methods and systems for partitioning barcode carrying beads into droplets are provided in US. Patent Publication Nos. 2019/0367997 and 2019/0064173, and International Application Nos. PCT/US20/17785 and PCT/US20/020486.

Beneficially, a discrete droplet partitioning a biological particle and a barcode carrying bead can effectively allow the attribution of the barcode to macromolecular constituents of the biological particle within the partition. The contents of a partition can remain discrete from the contents of other partitions.

In operation, the barcoded oligonucleotides can be released (e.g., in a partition), as described elsewhere herein. Alternatively, the nucleic acid molecules bound to the bead (e.g., gel bead) can be used to hybridize and capture analytes (e.g., one or more types of analytes) on the solid phase of the bead.

In some examples, beads, biological particles (e.g., labelled B cells) and droplets can flow along channels (e.g., the channels of a microfluidic device), in some cases at substantially regular flow profiles (e.g., at regular flow rates). Such regular flow profiles can permit a droplet to include a single bead and a single biological particle. Such regular flow profiles can permit the droplets to have an occupancy (e.g., droplets having beads and biological particles) greater than 5%, 10%, 20%, 30%, 40%, 50%, 60%, 70%, 80%, 90%, or 95%. Such regular flow profiles and devices that can be used to provide such regular flow profiles are provided in, for example, U.S. Patent Publication No. 2015/0292988.

A bead can be porous, non-porous, solid, semi-solid, semi-fluidic, fluidic, and/or a combination thereof. In some instances, a bead can be dissolvable, disruptable, and/or degradable. In some cases, a bead cannot be degradable. In some cases, the bead can be a gel bead. A gel bead can be a hydrogel bead. A gel bead can be formed from molecular precursors, such as a polymeric or monomeric species. A semi-solid bead can be a liposomal bead. Solid beads can include metals including iron oxide, gold, and silver. In some cases, the bead can be a silica bead. In some cases, the bead can be rigid. In other cases, the bead can be flexible and/or compressible.

A bead can be of any suitable shape. Examples of bead shapes include, but are not limited to, spherical, non-spherical, oval, oblong, amorphous, circular, cylindrical, and variations thereof.

Beads can be of uniform size or heterogeneous size. In some cases, the diameter of a bead can be at least about 10 nanometers (nm), 100 nm, 500 nm, 1 micrometer (μm), 5 μm, 10 μm, 20 μm, 30 μm, 40 μm, 50 μm, 60 μm, 70 μm, 80 μm, 90 μm, 100 μm, 250 μm, 500 μm, 1 mm, or greater. In some cases, a bead can have a diameter of less than about 10 nm, 100 nm, 500 nm, 1 μm, 5 μm, 10 μm, 20 μm, 30 μm, 40 μm, 50 μm, 60 μm, 70 μm, 80 μm, 90 μm, 100 μm, 250 μm, 500 μm, 1 mm, or less. In some cases, a bead can have a diameter in the range of about 40-75 μm, 30-75 μm, 20-75 μm, 40-85 μm, 40-95 μm, 20-100 μm, 10-100 μm, 1-100 μm, 20-250 μm, or 20-500 μm.

In certain aspects, beads can be provided as a population or plurality of beads having a relatively monodisperse size distribution. Where it may be desirable to provide relatively consistent amounts of reagents within partitions, maintaining relatively consistent bead characteristics, such as size, can contribute to the overall consistency. In some embodiments, the beads described herein can have size distributions that have a coefficient of variation in their cross-sectional dimensions of less than 50%, less than 40%, less than 30%, less than 20%, and in some cases less than 15%, less than 10%, less than 5%, or less.

A bead can include natural and/or synthetic materials. For example, a bead can include a natural polymer, a synthetic polymer or both natural and synthetic polymers. Examples of natural polymers include proteins and sugars such as deoxyribonucleic acid, rubber, cellulose, starch (e.g., amylose, amylopectin), proteins, enzymes, polysaccharides, silks, polyhydroxyalkanoates, chitosan, dextran, collagen, carrageenan, ispaghula, acacia, agar, gelatin, shellac, sterculia gum, xanthan gum, Corn sugar gum, guar gum, gum karaya, agarose, alginic acid, alginate, or natural polymers thereof. Examples of synthetic polymers include acrylics, nylons, silicones, spandex, viscose rayon, polycarboxylic acids, polyvinyl acetate, polyacrylamide, polyacrylate, polyethylene glycol, polyurethanes, polylactic acid, silica, polystyrene, polyacrylonitrile, polybutadiene, polycarbonate, polyethylene, polyethylene terephthalate, poly(chlorotrifluoroethylene), poly(ethylene oxide), poly(ethylene terephthalate), polyethylene, polyisobutylene, poly(methyl methacrylate), poly(oxymethylene), polyformaldehyde, polypropylene, polystyrene, poly(tetrafluoroethylene), poly(vinyl acetate), poly(vinyl alcohol), poly(vinyl chloride), poly(vinylidene dichloride), poly(vinylidene difluoride), poly(vinyl fluoride) and/or combinations (e.g., co-polymers) thereof. Beads can also be formed from materials other than polymers, including lipids, micelles, ceramics, glass-ceramics, material composites, metals, other inorganic materials, and others.

In some embodiments, the bead can contain molecular precursors (e.g., monomers or polymers), which can form a polymer network via polymerization of the molecular precursors. In some cases, a precursor can be an already polymerized species capable of undergoing further polymerization via, for example, a chemical cross-linkage. In some embodiments, a precursor can include one or more of an acrylamide or a methacrylamide monomer, oligomer, or polymer. In some cases, the bead can include prepolymers, which are oligomers capable of further polymerization. For example, polyurethane beads can be prepared using prepolymers. In some embodiments, the bead can contain individual polymers that can be further polymerized together. In some cases, beads can be generated via polymerization of different precursors, such that they include mixed polymers, co-polymers, and/or block co-polymers. In some embodiments, the bead can include covalent or ionic bonds between polymeric precursors (e.g., monomers, oligomers, linear polymers), nucleic acid molecules (e.g., oligonucleotides), primers, and other entities. In some embodiments, the covalent bonds can be carbon-carbon bonds, thioether bonds, or carbon-heteroatom bonds.

Cross-linking can be permanent or reversible, depending upon the particular cross-linker used. Reversible cross-linking can allow for the polymer to linearize or dissociate under appropriate conditions. In some embodiments, reversible cross-linking can also allow for reversible attachment of a material bound to the surface of a bead. In some embodiments, a cross-linker can form disulfide linkages. In some embodiments, the chemical cross-linker forming disulfide linkages can be cystamine or a modified cystamine.

In some embodiments, disulfide linkages can be formed between molecular precursor units (e.g., monomers, oligomers, or linear polymers) or precursors incorporated into a bead and nucleic acid molecules (e.g., oligonucleotides). Cystamine (including modified cystamines), for example, is an organic agent including a disulfide bond that can be used as a crosslinker agent between individual monomeric or polymeric precursors of a bead. Polyacrylamide can be polymerized in the presence of cystamine or a species including cystamine (e.g., a modified cystamine) to generate polyacrylamide gel beads including disulfide linkages (e.g., chemically degradable beads including chemically-reducible cross-linkers). The disulfide linkages can permit the bead to be degraded (or dissolved) upon exposure of the bead to a reducing agent.

In some embodiments, chitosan, a linear polysaccharide polymer, can be crosslinked with glutaraldehyde via hydrophilic chains to form a bead. Crosslinking of chitosan polymers can be achieved by chemical reactions that are initiated by heat, pressure, change in pH, and/or radiation.

In some embodiments, a bead can include an acrydite moiety, which in certain aspects can be used to attach one or more nucleic acid molecules (e.g., barcode sequence, barcoded nucleic acid molecule, barcoded oligonucleotide, primer, or other oligonucleotide) to the bead. In some cases, an acrydite moiety can refer to an acrydite analogue generated from the reaction of acrydite with one or more species, such as, the reaction of acrydite with other monomers and cross-linkers during a polymerization reaction. Acrydite moieties can be modified to form chemical bonds with a species to be attached, such as a nucleic acid molecule (e.g., barcode sequence, barcoded nucleic acid molecule, barcoded oligonucleotide, primer, or other oligonucleotide). Acrydite moieties can be modified with thiol groups capable of forming a disulfide bond or can be modified with groups already including a disulfide bond. The thiol or disulfide (via disulfide exchange) can be used as an anchor point for a species to be attached or another part of the acrydite moiety can be used for attachment. In some cases, attachment can be reversible, such that when the disulfide bond is broken (e.g., in the presence of a reducing agent), the attached species is released from the bead. In other cases, an acrydite moiety can include a reactive hydroxyl group that can be used for attachment.

Functionalization of beads for attachment of nucleic acid molecules (e.g., oligonucleotides) can be achieved through a wide range of different approaches, including activation of chemical groups within a polymer, incorporation of active or activatable functional groups in the polymer structure, or attachment at the pre-polymer or monomer stage in bead production.

For example, precursors (e.g., monomers, cross-linkers) that are polymerized to form a bead can include acrydite moieties, such that when a bead is generated, the bead also includes acrydite moieties. The acrydite moieties can be attached to a nucleic acid molecule (e.g., oligonucleotide), which can include a priming sequence (e.g., a primer for amplifying target nucleic acids, random primer, primer sequence for messenger RNA) and/or one or more barcode sequences. The one or more barcode sequences can include sequences that are the same for all nucleic acid molecules coupled to a given bead and/or sequences that are different across all nucleic acid molecules coupled to the given bead. The nucleic acid molecule can be incorporated into the bead.

In some embodiments, the nucleic acid molecule can include a functional sequence, for example, for attachment to a sequencing flow cell, such as, for example, a P5 sequence for Illumina® sequencing. In some cases, the nucleic acid molecule or derivative thereof (e.g., oligonucleotide or polynucleotide generated from the nucleic acid molecule) can include another functional sequence, such as, for example, a P7 sequence for attachment to a sequencing flow cell for Illumina sequencing. In some cases, the nucleic acid molecule can include a barcode sequence. In some cases, the primer can further include a unique molecular identifier (UMI). In some cases, the primer can include an R1 primer sequence for Illumina sequencing. In some cases, the primer can include an R2 primer sequence for Illumina sequencing. Examples of such nucleic acid molecules (e.g., oligonucleotides, polynucleotides, etc.) and uses thereof, as can be used with compositions, devices, methods and systems of the present disclosure, are provided in U.S. Patent Pub. Nos. 2014/0378345 and 2015/0376609.

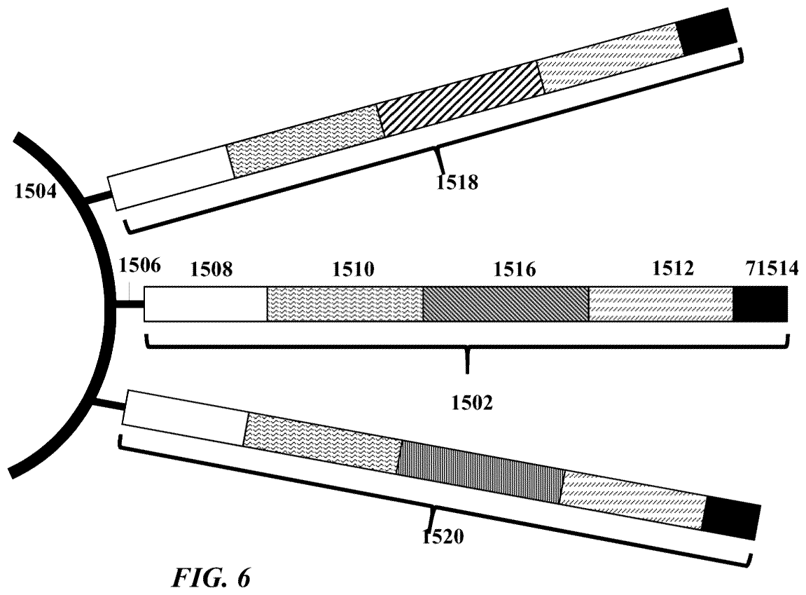

FIG. 6 illustrates an example of a barcode carrying bead. A nucleic acid molecule 1502, such as an oligonucleotide, can be coupled to a bead 1504 by a releasable linkage 1506, such as, for example, a disulfide linker. The same bead 1504 can be coupled (e.g., via releasable linkage) to one or more other nucleic acid molecules 1518, 1520. The nucleic acid molecule 1502 can be or include a barcode. As noted elsewhere herein, the structure of the barcode can include a number of sequence elements. The nucleic acid molecule 1502 can include a functional sequence 1508 that can be used in subsequent processing. For example, the functional sequence 1508 can include one or more of a sequencer specific flow cell attachment sequence (e.g., a P5 sequence for Illumina® sequencing systems) and a sequencing primer sequence (e.g., a R1 primer for Illumina® sequencing systems). The nucleic acid molecule 1502 can include a barcode sequence 1510 for use in barcoding the sample (e.g., DNA, RNA, protein, etc.). In some cases, the barcode sequence 1510 can be bead-specific such that the barcode sequence 1510 is common to all nucleic acid molecules (e.g., including nucleic acid molecule 1502) coupled to the same bead 1504. Alternatively or in addition, the barcode sequence 1510 can be partition-specific such that the barcode sequence 1510 is common to all nucleic acid molecules coupled to one or more beads that are partitioned into the same partition. The nucleic acid molecule 1502 can include a specific priming sequence 1512, such as an mRNA specific priming sequence (e.g., poly-T sequence), a targeted priming sequence, and/or a random priming sequence. The nucleic acid molecule 1502 can include an anchoring sequence 1514 to ensure that the specific priming sequence 1512 hybridizes at the sequence end (e.g., of the mRNA). For example, the anchoring sequence 1514 can include a random short sequence of nucleotides, such as a 1-mer, 2-mer, 3-mer or longer sequence, which can ensure that a poly-T segment is more likely to hybridize at the sequence end of the poly-A tail of the mRNA.

The nucleic acid molecule 1502 can include a unique molecular identifying sequence 1516 (e.g., unique molecular identifier (UMI)). In some cases, the unique molecular identifying sequence 1516 can include from about 5 to about 8 nucleotides. Alternatively, the unique molecular identifying sequence 1516 can compress less than about 5 or more than about 8 nucleotides. The unique molecular identifying sequence 1516 can be a unique sequence that varies across individual nucleic acid molecules (e.g., 1502, 1518, 1520, etc.) coupled to a single bead (e.g., bead 1504). In some cases, the unique molecular identifying sequence 1516 can be a random sequence (e.g., such as a random N-mer sequence). For example, the UMI can provide a unique identifier of the starting mRNA molecule that was captured, in order to allow quantitation of the number of original expressed RNA. As will be appreciated, although FIG. 15 shows three nucleic acid molecules 1502, 1518, 1520 coupled to the surface of the bead 1504, an individual bead can be coupled to any number of individual nucleic acid molecules, for example, from one to tens to hundreds of thousands or even millions of individual nucleic acid molecules. The respective barcodes for the individual nucleic acid molecules can include both common sequence segments or relatively common sequence segments (e.g., 1508, 1510, 1512, etc.) and variable or unique sequence segments (e.g., 1516) between different individual nucleic acid molecules coupled to the same bead.

In operation, a biological particle (e.g., cell, DNA, RNA, etc.) can be co-partitioned along with a barcode bearing bead 1504. The nucleic acid barcode molecules 1502, 1518, 1520 can be released from the bead 1504 in the partition. By way of example, in the context of analyzing sample RNA, the poly-T segment (e.g., 1512) of one of the released nucleic acid molecules (e.g., 1502) can hybridize to the poly-A tail of a mRNA molecule. Reverse transcription can result in a cDNA transcript of the mRNA, but which transcript includes each of the sequence segments 1508, 1510, 1516 of the nucleic acid molecule 1502. Because the nucleic acid molecule 1502 includes an anchoring sequence 1514, it will more likely hybridize to and prime reverse transcription at the sequence end of the poly-A tail of the mRNA. Within any given partition, all of the cDNA transcripts of the individual mRNA molecules can include a common barcode sequence segment 1510. However, the transcripts made from the different mRNA molecules within a given partition can vary at the unique molecular identifying sequence 1512 segment (e.g., UMI segment). Beneficially, even following any subsequent amplification of the contents of a given partition, the number of different UMIs can be indicative of the quantity of mRNA originating from a given partition, and thus from the biological particle (e.g., cell). As noted above, the transcripts can be amplified, cleaned up and sequenced to identify the sequence of the cDNA transcript of the mRNA, as well as to sequence the barcode segment and the UMI segment. While a poly-T primer sequence is described, other targeted or random priming sequences can also be used in priming the reverse transcription reaction. Likewise, although described as releasing the barcoded oligonucleotides into the partition, in some cases, the nucleic acid molecules bound to the bead (e.g., gel bead) can be used to hybridize and capture the mRNA on the solid phase of the bead, for example, in order to facilitate the separation of the RNA from other cell contents. In such cases, further processing can be performed, in the partitions or outside the partitions (e.g., in bulk). For instance, the RNA molecules on the beads can be subjected to reverse transcription or other nucleic acid processing, additional adapter sequences can be added to the barcoded nucleic acid molecules, or other nucleic acid reactions (e.g., amplification, nucleic acid extension) can be performed. The beads or products thereof (e.g., barcoded nucleic acid molecules) can be collected from the partitions, and/or pooled together and subsequently subjected to clean up and further characterization (e.g., sequencing).

The operations described herein can be performed at any useful or suitable step. For instance, the beads including nucleic acid barcode molecules can be introduced into a partition (e.g., well or droplet) prior to, during, or following introduction of a sample into the partition. The nucleic acid molecules of a sample can be subjected to barcoding, which can occur on the bead (in cases where the nucleic acid molecules remain coupled to the bead) or following release of the nucleic acid barcode molecules into the partition. In cases where analytes from the sample are captured by the nucleic acid barcode molecules in a partition (e.g., by hybridization), captured analytes from various partitions may be collected, pooled, and subjected to further processing (e.g., reverse transcription, adapter attachment, amplification, clean up, sequencing). For example, in cases wherein the nucleic acid molecules from the sample remain attached to the bead, the beads from various partitions can be collected, pooled, and subjected to further processing (e.g., reverse transcription, adapter attachment, amplification, clean up, and/or sequencing). In other instances, one or more of the processing methods, e.g., reverse transcription, can occur in the partition. For example, conditions sufficient for barcoding, adapter attachment, reverse transcription, or other nucleic acid processing operations can be provided in the partition and performed prior to clean up and sequencing.

In some instances, a bead can include a capture sequence or binding sequence configured to bind to a corresponding capture sequence or binding sequence. In some instances, a bead can include a plurality of different capture sequences or binding sequences configured to bind to different respective corresponding capture sequences or binding sequences. For example, a bead can include a first subset of one or more capture sequences each configured to bind to a first corresponding capture sequence, a second subset of one or more capture sequences each configured to bind to a second corresponding capture sequence, a third subset of one or more capture sequences each configured to bind to a third corresponding capture sequence, and etc. A bead can include any number of different capture sequences. In some instances, a bead can include at least 2, 3, 4, 5, 6, 7, 8, 9, 10 or more different capture sequences or binding sequences configured to bind to different respective capture sequences or binding sequences, respectively. Alternatively or in addition, a bead can include at most about 10, 9, 8, 7, 6, 5, 4, 3, or 2 different capture sequences or binding sequences configured to bind to different respective capture sequences or binding sequences. In some instances, the different capture sequences or binding sequences can be configured to facilitate analysis of a same type of analyte. In some instances, the different capture sequences or binding sequences can be configured to facilitate analysis of different types of analytes (with the same bead). The capture sequence can be designed to attach to a corresponding capture sequence. Beneficially, such corresponding capture sequence can be introduced to, or otherwise induced in, a biological particle (e.g., cell, cell bead, etc.) for performing different assays in various formats (e.g., barcoded antibodies including the corresponding capture sequence, barcoded MHC dextramers including the corresponding capture sequence, barcoded guide RNA molecules including the corresponding capture sequence, etc.), such that the corresponding capture sequence can later interact with the capture sequence associated with the bead. In some instances, a capture sequence coupled to a bead (or other support) can be configured to attach to a linker molecule, such as a splint molecule, wherein the linker molecule is configured to couple the bead (or other support) to other molecules through the linker molecule, such as to one or more analytes or one or more other linker molecules.

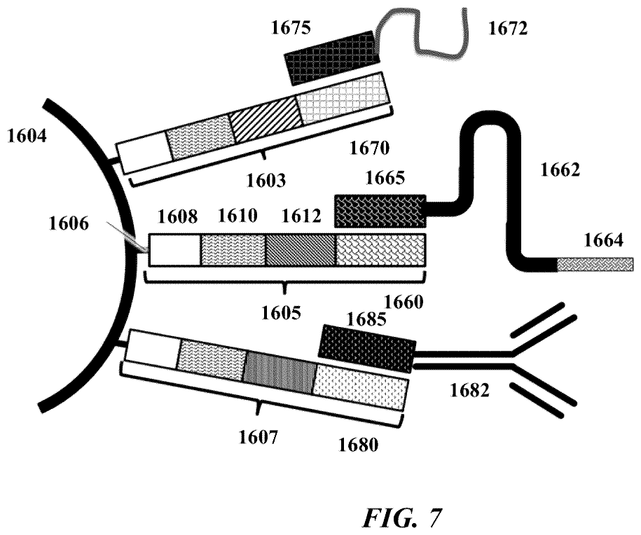

FIG. 7 illustrates a non-limiting example of a barcode carrying bead in accordance with some embodiments of the disclosure. A nucleic acid molecule 1605, such as an oligonucleotide, can be coupled to a bead 1604 by a releasable linkage 1606, such as, for example, a disulfide linker. The nucleic acid molecule 1605 can include a first capture sequence 1660. The same bead 1604 can be coupled, e.g., via releasable linkage, to one or more other nucleic acid molecules 1603, 1607 including other capture sequences. The nucleic acid molecule 1605 can be or include a barcode. As described elsewhere herein, the structure of the barcode can include a number of sequence elements, such as a functional sequence 1608 (e.g., flow cell attachment sequence, sequencing primer sequence, etc.), a barcode sequence 1610 (e.g., bead-specific sequence common to bead, partition-specific sequence common to partition, etc.), and a unique molecular identifier 1612 (e.g., unique sequence within different molecules attached to the bead), or partial sequences thereof. The capture sequence 1660 can be configured to attach to a corresponding capture sequence 1665 (e.g., capture handle). In some instances, the corresponding capture sequence 1665 can be coupled to another molecule that can be an analyte or an intermediary carrier. For example, as illustrated in FIG. 7, the corresponding capture sequence 1665 is coupled to a guide RNA molecule 1662 including a target sequence 1664, wherein the target sequence 1664 is configured to attach to the analyte. Another oligonucleotide molecule 1607 attached to the bead 1604 includes a second capture sequence 1680 which is configured to attach to a second corresponding capture sequence (e.g., capture handle) 1685. As illustrated in FIG. 7, the second corresponding capture sequence 1685 is coupled to an antibody 1682. In some cases, the antibody 1682 can have binding specificity to an analyte (e.g., surface protein). Alternatively, the antibody 1682 cannot have binding specificity. Another oligonucleotide molecule 1603 attached to the bead 1604 includes a third capture sequence 470 which is configured to attach to a second corresponding capture sequence 1675. As illustrated in FIG. 7, the third corresponding capture sequence (e.g., capture handle) 1675 is coupled to a molecule 1672. The molecule 1672 may or may not be configured to target an analyte. The other oligonucleotide molecules 1603, 1607 can include the other sequences (e.g., functional sequence, barcode sequence, UMI, etc.) described with respect to oligonucleotide molecule 1605. While a single oligonucleotide molecule including each capture sequence is illustrated in FIG. 7, it will be appreciated that, for each capture sequence, the bead can include a set of one or more oligonucleotide molecules each including the capture sequence. For example, the bead can include any number of sets of one or more different capture sequences. Alternatively or in addition, the bead 1604 can include other capture sequences. Alternatively or in addition, the bead 1604 can include fewer types of capture sequences (e.g., two capture sequences). Alternatively or in addition, the bead 1604 can include oligonucleotide molecule(s) including a priming sequence, such as a specific priming sequence such as an mRNA specific priming sequence (e.g., poly-T sequence), a targeted priming sequence, and/or a random priming sequence, for example, to facilitate an assay for gene expression.

The generation of a barcoded sequence, see, e.g., FIG. 6, is described herein. In some embodiments, precursors including a functional group that is reactive or capable of being activated such that it becomes reactive can be polymerized with other precursors to generate gel beads including the activated or activatable functional group. The functional group can then be used to attach additional species (e.g., disulfide linkers, primers, other oligonucleotides, etc.) to the gel beads. For example, some precursors including a carboxylic acid (COOH) group can co-polymerize with other precursors to form a gel bead that also includes a COOH functional group. In some cases, acrylic acid (a species including free COOH groups), acrylamide, and bis(acryloyl) cystamine can be co-polymerized together to generate a gel bead including free COOH groups. The COOH groups of the gel bead can be activated (e.g., via 1-Ethyl-3-(3-dimethylaminopropyl) carbodiimide (EDC) and N-Hydroxysuccinimide (NHS) or 4-(4,6-Dimethoxy-1,3,5-triazin-2-yl)-4-methylmorpholinium chloride (DMTMM)) such that they are reactive (e.g., reactive to amine functional groups where EDC/NHS or DMTMM are used for activation). The activated COOH groups can then react with an appropriate species (e.g., a species including an amine functional group where the carboxylic acid groups are activated to be reactive with an amine functional group) including a moiety to be linked to the bead.

Beads including disulfide linkages in their polymeric network can be functionalized with additional species via reduction of some of the disulfide linkages to free thiols. The disulfide linkages can be reduced via, for example, the action of a reducing agent (e.g., DTT, TCEP, etc.) to generate free thiol groups, without dissolution of the bead. Free thiols of the beads can then react with free thiols of a species or a species including another disulfide bond (e.g., via thiol-disulfide exchange) such that the species can be linked to the beads (e.g., via a generated disulfide bond). In some cases, free thiols of the beads can react with any other suitable group. For example, free thiols of the beads can react with species including an acrydite moiety. The free thiol groups of the beads can react with the acrydite via Michael addition chemistry, such that the species including the acrydite is linked to the bead. In some cases, uncontrolled reactions can be prevented by inclusion of a thiol capping agent such as N-ethylmaleimide or iodoacetate.

Activation of disulfide linkages within a bead can be controlled such that only a small number of disulfide linkages are activated. Control can be exerted, for example, by controlling the concentration of a reducing agent used to generate free thiol groups and/or concentration of reagents used to form disulfide bonds in bead polymerization. In some cases, a low concentration (e.g., molecules of reducing agent: gel bead ratios of less than or equal to about 1:100,000,000,000, less than or equal to about 1:10,000,000,000, less than or equal to about 1:1,000,000,000, less than or equal to about 1:100,000,000, less than or equal to about 1:10,000,000, less than or equal to about 1:1,000,000, less than or equal to about 1:100,000, less than or equal to about 1:10,000) of reducing agent can be used for reduction. Controlling the number of disulfide linkages that are reduced to free thiols can be useful in ensuring bead structural integrity during functionalization. In some cases, optically-active agents, such as fluorescent dyes can be coupled to beads via free thiol groups of the beads and used to quantify the number of free thiols present in a bead and/or track a bead.

In some embodiments, addition of moieties to a gel bead after gel bead formation can be advantageous. For example, addition of an oligonucleotide (e.g., barcoded oligonucleotide, such as a barcoded nucleic acid molecule) after gel bead formation can avoid loss of the species during chain transfer termination that can occur during polymerization. Moreover, smaller precursors (e.g., monomers or cross linkers that do not include side chain groups and linked moieties) can be used for polymerization and can be minimally hindered from growing chain ends due to viscous effects. In some cases, functionalization after gel bead synthesis can minimize exposure of species (e.g., oligonucleotides) to be loaded with potentially damaging agents (e.g., free radicals) and/or chemical environments. In some cases, the generated gel can possess an upper critical solution temperature (UCST) that can permit temperature driven swelling and collapse of a bead. Such functionality can aid in oligonucleotide (e.g., a primer) infiltration into the bead during subsequent functionalization of the bead with the oligonucleotide. Post-production functionalization can also be useful in controlling loading ratios of species in beads, such that, for example, the variability in loading ratio is minimized. Species loading can also be performed in a batch process such that a plurality of beads can be functionalized with the species in a single batch.

A bead injected or otherwise introduced into a partition can include releasably, cleavably, or reversibly attached barcodes (e.g., partition barcode sequences). A bead injected or otherwise introduced into a partition can include activatable barcodes. A bead injected or otherwise introduced into a partition can be degradable, disruptable, or dissolvable beads.

Barcodes can be releasably, cleavably or reversibly attached to the beads such that barcodes can be released or be releasable through cleavage of a linkage between the barcode molecule and the bead, or released through degradation of the underlying bead itself, allowing the barcodes to be accessed or be accessible by other reagents, or both. In non-limiting examples, cleavage can be achieved through reduction of di-sulfide bonds, use of restriction enzymes, photo-activated cleavage, or cleavage via other types of stimuli (e.g., chemical, thermal, pH, enzymatic, etc.) and/or reactions, such as described elsewhere herein. Releasable barcodes can sometimes be referred to as being activatable, in that they are available for reaction once released. Thus, for example, an activatable barcode can be activated by releasing the barcode from a bead (or other suitable type of partition described herein). Other activatable configurations are also envisioned in the context of the described methods and systems.

In addition to, or as an alternative to the cleavable linkages between the beads and the associated molecules, such as barcode containing nucleic acid molecules (e.g., barcoded oligonucleotides), the beads can be degradable, disruptable, or dissolvable spontaneously or upon exposure to one or more stimuli (e.g., temperature changes, pH changes, exposure to particular chemical species or phase, exposure to light, reducing agent, etc.). In some cases, a bead can be dissolvable, such that material components of the beads are solubilized when exposed to a particular chemical species or an environmental change, such as a change temperature or a change in pH. In some cases, a gel bead can be degraded or dissolved at elevated temperature and/or in basic conditions. In some cases, a bead can be thermally degradable such that when the bead is exposed to an appropriate change in temperature (e.g., heat), the bead degrades. Degradation or dissolution of a bead bound to a species (e.g., a nucleic acid molecule, e.g., barcoded oligonucleotide) can result in release of the species from the bead.

As will be appreciated from the above disclosure, the degradation of a bead can refer to the disassociation of a bound (e.g., capture agent configured to couple to a secreted antibody or antigen-binding fragment thereof) or entrained species (e.g., labelled T cells or CAR-T cells) from a bead, both with and without structurally degrading the physical bead itself. For example, the degradation of the bead can involve cleavage of a cleavable linkage via one or more species and/or methods described elsewhere herein. In another example, entrained species can be released from beads through osmotic pressure differences due to, for example, changing chemical environments. By way of example, alteration of bead pore sizes due to osmotic pressure differences can generally occur without structural degradation of the bead itself. In some cases, an increase in pore size due to osmotic swelling of a bead can permit the release of entrained species within the bead. In other cases, osmotic shrinking of a bead can cause a bead to better retain an entrained species due to pore size contraction.

A degradable bead can be introduced into a partition, such as a droplet of an emulsion or a well, such that the bead degrades within the partition and any associated species (e.g., oligonucleotides) are released within the droplet when the appropriate stimulus is applied. The free species (e.g., oligonucleotides, nucleic acid molecules) can interact with other reagents contained in the partition. For example, a polyacrylamide bead including cystamine and linked, via a disulfide bond, to a barcode sequence, can be combined with a reducing agent within a droplet of a water-in-oil emulsion. Within the droplet, the reducing agent can break the various disulfide bonds, resulting in bead degradation and release of the barcode sequence into the aqueous, inner environment of the droplet. In another example, heating of a droplet including a bead-bound barcode sequence in basic solution can also result in bead degradation and release of the attached barcode sequence into the aqueous, inner environment of the droplet.

Any suitable number of molecular tag molecules (e.g., primer, barcoded oligonucleotide) can be associated with a bead such that, upon release from the bead, the molecular tag molecules (e.g., primer, e.g., barcoded oligonucleotide) are present in the partition at a pre-defined concentration. Such pre-defined concentration can be selected to facilitate certain reactions for generating a sequencing library, e.g., amplification, within the partition. In some cases, the pre-defined concentration of the primer can be limited by the process of producing nucleic acid molecule (e.g., oligonucleotide) bearing beads.

In some cases, beads can be non-covalently loaded with one or more reagents. The beads can be non-covalently loaded by, for instance, subjecting the beads to conditions sufficient to swell the beads, allowing sufficient time for the reagents to diffuse into the interiors of the beads, and subjecting the beads to conditions sufficient to de-swell the beads. The swelling of the beads can be accomplished, for instance, by placing the beads in a thermodynamically favorable solvent, subjecting the beads to a higher or lower temperature, subjecting the beads to a higher or lower ion concentration, and/or subjecting the beads to an electric field. The swelling of the beads can be accomplished by various swelling methods. The de-swelling of the beads can be accomplished, for instance, by transferring the beads in a thermodynamically unfavorable solvent, subjecting the beads to lower or high temperatures, subjecting the beads to a lower or higher ion concentration, and/or removing an electric field. The de-swelling of the beads can be accomplished by various de-swelling methods. Transferring the beads can cause pores in the bead to shrink. The shrinking can then hinder reagents within the beads from diffusing out of the interiors of the beads. The hindrance can be due to steric interactions between the reagents and the interiors of the beads. The transfer can be accomplished microfluidically. For instance, the transfer can be achieved by moving the beads from one co-flowing solvent stream to a different co-flowing solvent stream. The swellability and/or pore size of the beads can be adjusted by changing the polymer composition of the bead.

In some cases, an acrydite moiety linked to a precursor, another species linked to a precursor, or a precursor itself can include a labile bond, such as chemically, thermally, or photo-sensitive bond e.g., disulfide bond, UV sensitive bond, or the like. Once acrydite moieties or other moieties including a labile bond are incorporated into a bead, the bead can also include the labile bond. The labile bond can be, for example, useful in reversibly linking (e.g., covalently linking) species (e.g., barcodes, primers, etc.) to a bead. In some cases, a thermally labile bond can include a nucleic acid hybridization based attachment, e.g., where an oligonucleotide is hybridized to a complementary sequence that is attached to the bead, such that thermal melting of the hybrid releases the oligonucleotide, e.g., a barcode containing sequence, from the bead.

The addition of multiple types of labile bonds to a gel bead can result in the generation of a bead capable of responding to varied stimuli. Each type of labile bond can be sensitive to an associated stimulus (e.g., chemical stimulus, light, temperature, enzymatic, etc.) such that release of species attached to a bead via each labile bond can be controlled by the application of the appropriate stimulus. Such functionality can be useful in controlled release of species from a gel bead. In some cases, another species including a labile bond can be linked to a gel bead after gel bead formation via, for example, an activated functional group of the gel bead as described above. As will be appreciated, barcodes that are releasably, cleavably or reversibly attached to the beads described herein include barcodes that are released or releasable through cleavage of a linkage between the barcode molecule and the bead, or that are released through degradation of the underlying bead itself, allowing the barcodes to be accessed or accessible by other reagents, or both.

The barcodes that are releasable as described herein can sometimes be referred to as being activatable, in that they are available for reaction once released. Thus, for example, an activatable barcode can be activated by releasing the barcode from a bead (or other suitable type of partition described herein). Other activatable configurations are also envisioned in the context of the described methods and systems.

In addition to thermally cleavable bonds, disulfide bonds and UV sensitive bonds, other non-limiting examples of labile bonds that can be coupled to a precursor or bead include an ester linkage (e.g., cleavable with an acid, a base, or hydroxylamine), a vicinal diol linkage (e.g., cleavable via sodium periodate), a Diels-Alder linkage (e.g., cleavable via heat), a sulfone linkage (e.g., cleavable via a base), a silyl ether linkage (e.g., cleavable via an acid), a glycosidic linkage (e.g., cleavable via an amylase), a peptide linkage (e.g., cleavable via a protease), or a phosphodiester linkage (e.g., cleavable via a nuclease (e.g., DNAase)). A bond can be cleavable via other nucleic acid molecule targeting enzymes, such as restriction enzymes (e.g., restriction endonucleases), as described further below.

Species can be encapsulated in beads (e.g., capture agent) during bead generation (e.g., during polymerization of precursors). Such species may or may not participate in polymerization. Such species can be entered into polymerization reaction mixtures such that generated beads include the species upon bead formation. In some cases, such species can be added to the gel beads after formation. Such species can include, for example, nucleic acid molecules (e.g., oligonucleotides), reagents for a nucleic acid amplification reaction (e.g., primers, polymerases, dNTPs, co-factors (e.g., ionic co-factors, buffers) including those described herein, reagents for enzymatic reactions (e.g., enzymes, co-factors, substrates, buffers), reagents for nucleic acid modification reactions such as polymerization, ligation, or digestion, and/or reagents for template preparation (e.g., tagmentation) for one or more sequencing platforms (e.g., Nextera® for Illumina®). Such species can include one or more enzymes described herein, including without limitation, polymerase, reverse transcriptase, restriction enzymes (e.g., endonuclease), transposase, ligase, proteinase K, DNAse, etc. Such species can include one or more reagents described elsewhere herein (e.g., lysis agents, inhibitors, inactivating agents, chelating agents, stimulus). Trapping of such species can be controlled by the polymer network density generated during polymerization of precursors, control of ionic charge within the gel bead (e.g., via ionic species linked to polymerized species), or by the release of other species. Encapsulated species can be released from a bead upon bead degradation and/or by application of a stimulus capable of releasing the species from the bead. Alternatively or in addition, species can be partitioned in a partition (e.g., droplet) during or subsequent to partition formation. Such species can include, without limitation, the abovementioned species that can also be encapsulated in a bead.

A degradable bead can include one or more species with a labile bond such that, when the bead/species is exposed to the appropriate stimuli, the bond is broken and the bead degrades. The labile bond can be a chemical bond (e.g., covalent bond, ionic bond) or can be another type of physical interaction (e.g., van der Waals interactions, dipole-dipole interactions, etc.). In some cases, a crosslinker used to generate a bead can include a labile bond. Upon exposure to the appropriate conditions, the labile bond can be broken and the bead degraded. For example, upon exposure of a polyacrylamide gel bead including cystamine crosslinkers to a reducing agent, the disulfide bonds of the cystamine can be broken and the bead degraded.

A degradable bead can be useful in more quickly releasing an attached species (e.g., a nucleic acid molecule, a barcode sequence, a primer, etc.) from the bead when the appropriate stimulus is applied to the bead as compared to a bead that does not degrade. For example, for a species bound to an inner surface of a porous bead or in the case of an encapsulated species, the species can have greater mobility and accessibility to other species in solution upon degradation of the bead. In some cases, a species can also be attached to a degradable bead via a degradable linker (e.g., disulfide linker). The degradable linker can respond to the same stimuli as the degradable bead or the two degradable species can respond to different stimuli. For example, a barcode sequence can be attached, via a disulfide bond, to a polyacrylamide bead including cystamine. Upon exposure of the barcoded-bead to a reducing agent, the bead degrades and the barcode sequence is released upon breakage of both the disulfide linkage between the barcode sequence and the bead and the disulfide linkages of the cystamine in the bead.

As will be appreciated from the above disclosure, while referred to as degradation of a bead, in many instances as noted above, that degradation can refer to the disassociation of a bound or entrained species from a bead, both with and without structurally degrading the physical bead itself. For example, entrained species can be released from beads through osmotic pressure differences due to, for example, changing chemical environments. By way of example, alteration of bead pore sizes due to osmotic pressure differences can generally occur without structural degradation of the bead itself. In some cases, an increase in pore size due to osmotic swelling of a bead can permit the release of entrained species within the bead. In other cases, osmotic shrinking of a bead can cause a bead to better retain an entrained species due to pore size contraction.

Where degradable beads are provided, it can be beneficial to avoid exposing such beads to the stimulus or stimuli that cause such degradation prior to a given time, in order to, for example, avoid premature bead degradation and issues that arise from such degradation, including for example poor flow characteristics and aggregation. By way of example, where beads include reducible cross-linking groups, such as disulfide groups, it will be desirable to avoid contacting such beads with reducing agents, e.g., DTT or other disulfide cleaving reagents. In such cases, treatment to the beads described herein will, in some cases be provided free of reducing agents, such as DTT. Because reducing agents are often provided in commercial enzyme preparations, it can be desirable to provide reducing agent free (or DTT free) enzyme preparations in treating the beads described herein. Examples of such enzymes include, e.g., polymerase enzyme preparations, reverse transcriptase enzyme preparations, ligase enzyme preparations, as well as many other enzyme preparations that can be used to treat the beads described herein. The terms “reducing agent free” or “DTT free” preparations can refer to a preparation having less than about 1/10th, less than about 1/50th, or even less than about 1/100th of the lower ranges for such materials used in degrading the beads. For example, for DTT, the reducing agent free preparation can have less than about 0.01 millimolar (mM), 0.005 mM, 0.001 mM DTT, 0.0005 mM DTT, or even less than about 0.0001 mM DTT. In many cases, the amount of DTT can be undetectable.

Numerous chemical triggers can be used to trigger the degradation of beads. Examples of these chemical changes can include, but are not limited to pH-mediated changes to the integrity of a component within the bead, degradation of a component of a bead via cleavage of cross-linked bonds, and depolymerization of a component of a bead.

In some embodiments, a bead can be formed from materials that include degradable chemical crosslinkers, such as BAC or cystamine. Degradation of such degradable crosslinkers can be accomplished through a number of mechanisms. In some examples, a bead can be contacted with a chemical degrading agent that can induce oxidation, reduction or other chemical changes. For example, a chemical degrading agent can be a reducing agent, such as dithiothreitol (DTT). Additional examples of reducing agents can include β-mercaptocthanol, (2S)-2-amino-1,4-dimercaptobutane (dithiobutylamine or DTBA), tris(2-carboxyethyl) phosphine (TCEP), or combinations thereof. A reducing agent can degrade the disulfide bonds formed between gel precursors forming the bead, and thus, degrade the bead. In other cases, a change in pH of a solution, such as an increase in pH, can trigger degradation of a bead. In other cases, exposure to an aqueous solution, such as water, can trigger hydrolytic degradation, and thus degradation of the bead. In some cases, any combination of stimuli can trigger degradation of a bead. For example, a change in pH can enable a chemical agent (e.g., DTT) to become an effective reducing agent.

Beads can also be induced to release their contents upon the application of a thermal stimulus. A change in temperature can cause a variety of changes to a bead. For example, heat can cause a solid bead to liquefy. A change in heat can cause melting of a bead such that a portion of the bead degrades. In other cases, heat can increase the internal pressure of the bead components such that the bead ruptures or explodes. Heat can also act upon heat-sensitive polymers used as materials to construct beads.

Any suitable agent can degrade beads. In some embodiments, changes in temperature or pH can be used to degrade thermo-sensitive or pH-sensitive bonds within beads. In some embodiments, chemical degrading agents can be used to degrade chemical bonds within beads by oxidation, reduction or other chemical changes. For example, a chemical degrading agent can be a reducing agent, such as DTT, wherein DTT can degrade the disulfide bonds formed between a crosslinker and gel precursors, thus degrading the bead. In some embodiments, a reducing agent can be added to degrade the bead, which may or may not cause the bead to release its contents. Examples of reducing agents can include dithiothreitol (DTT), β-mercaptoethanol, (2S)-2-amino-1,4-dimercaptobutane (dithiobutylamine or DTBA), tris(2-carboxyethyl) phosphine (TCEP), or combinations thereof. The reducing agent can be present at a concentration of about 0.1 mM, 0.5 mM, 1 mM, 5 mM, or 10 mM. The reducing agent can be present at a concentration of at least about 0.1 mM, 0.5 mM, 1 mM, 5 mM, 10 mM, or greater than 10 mM. The reducing agent can be present at concentration of at most about 10 mM, 5 mM, 1 mM, 0.5 mM, 0.1 mM, or less.

Any suitable number of molecular tag molecules (e.g., primer, barcoded oligonucleotide) can be associated with a bead such that, upon release from the bead, the molecular tag molecules (e.g., primer, e.g., barcoded oligonucleotide) are present in the partition at a pre-defined concentration. Such pre-defined concentration can be selected to facilitate certain reactions for generating a sequencing library, e.g., amplification, within the partition. In some cases, the pre-defined concentration of the primer can be limited by the process of producing oligonucleotide bearing beads.

Although FIG. 4 and FIG. 5 have been described in terms of providing substantially singly occupied partitions, above, in certain cases, it may be desirable to provide multiply occupied partitions, e.g., containing two, three, four or more cells and/or beads including nucleic acid barcode molecules (e.g., oligonucleotides) within a single partition (e.g., multi-omics method described elsewhere, herein). Accordingly, as noted above, the flow characteristics of the biological particle and/or bead containing fluids and partitioning fluids can be controlled to provide for such multiply occupied partitions. In particular, the flow parameters can be controlled to provide a given occupancy rate at greater than about 50% of the partitions, greater than about 75%, and in some cases greater than about 80%, 90%, 95%, or higher.

In some cases, additional beads can be used to deliver additional reagents to a partition. In such cases, it can be advantageous to introduce different beads into a common channel or droplet generation junction, from different bead sources (e.g., containing different associated reagents) through different channel inlets into such common channel or droplet generation junction (e.g., junction 1210). In such cases, the flow and frequency of the different beads into the channel or junction can be controlled to provide for a certain ratio of beads from each source, while ensuring a given pairing or combination of such beads into a partition with a given number of biological particles (e.g., one biological particle and one bead per partition).

The partitions described herein can include small volumes, for example, less than about 10 microliters (μL), 5 μL, 1 μL, 900 picoliters (pL), 800 pL, 700 pL, 600 pL, 500 pL, 400 pL, 300 pL, 200 pL, 100 pL, 50 pL, 20 pL, 10 pL, 1 pL, 500 nanoliters (nL), 100 nL, 50 nL, or less.

For example, in the case of droplet based partitions, the droplets can have overall volumes that are less than about 1000 pL, 900 pL, 800 pL, 700 pL, 600 pL, 500 pL, 400 pL, 300 pL, 200 pL, 100 pL, 50 pL, 20 pL, 10 pL, 1 pL, or less. Where co-partitioned with beads, it will be appreciated that the sample fluid volume, e.g., including co-partitioned biological particles and/or beads, within the partitions can be less than about 90% of the above described volumes, less than about 80%, less than about 70%, less than about 60%, less than about 50%, less than about 40%, less than about 30%, less than about 20%, or less than about 10% of the above described volumes.

As is described elsewhere herein, partitioning species can generate a population or plurality of partitions. In such cases, any suitable number of partitions can be generated or otherwise provided. For example, at least about 1,000 partitions, at least about 5,000 partitions, at least about 10,000 partitions, at least about 50,000 partitions, at least about 100,000 partitions, at least about 500,000 partitions, at least about 1,000,000 partitions, at least about 5,000,000 partitions at least about 10,000,000 partitions, at least about 50,000,000 partitions, at least about 100,000,000 partitions, at least about 500,000,000 partitions, at least about 1,000,000,000 partitions, or more partitions can be generated or otherwise provided. Moreover, the plurality of partitions can include both unoccupied partitions (e.g., empty partitions) and occupied partitions.

### Reagents

In accordance with certain aspects, biological particles can be partitioned along with lysis reagents in order to release the contents of the biological particles within the partition. Scc, e.g., U.S. Pat. Pub. 2018/0216162 (now U.S. Pat. No. 10,428,326), U.S. Pat. Pub. 2019/0100632 (now U.S. Pat. No. 10,590,244), and U.S. Pat. Pub. 2019/0233878. Biological particles (e.g., cells, cell beads, cell nuclei, organelles, and the like) can be partitioned together with nucleic acid barcode molecules and the nucleic acid molecules of or derived from the biological particle (e.g., mRNA, cDNA, gDNA, etc.,) can be barcoded as described elsewhere herein. In some embodiments, biological particles are co-partitioned with barcode carrying beads (e.g., gel beads) and the nucleic acid molecules of or derived from the biological particle are barcoded as described elsewhere herein. In such cases, the lysis agents can be contacted with the biological particle suspension concurrently with, or immediately prior to, the introduction of the biological particles into the partitioning junction/droplet generation zone (e.g., junction 1210), such as through an additional channel or channels upstream of the channel junction. In accordance with other aspects, additionally or alternatively, biological particles can be partitioned along with other reagents, as will be described further below.

Beneficially, when lysis reagents and biological particles are co-partitioned, the lysis reagents can facilitate the release of the contents of the biological particles within the partition. The contents released in a partition can remain discrete from the contents of other partitions.

As will be appreciated, the channel segments described herein can be coupled to any of a variety of different fluid sources or receiving components, including reservoirs, tubing, manifolds, or fluidic components of other systems. As will be appreciated, the microfluidic channel structures can have other geometries and/or configurations. For example, a microfluidic channel structure can have more than two channel junctions. For example, a microfluidic channel structure can have 2, 3, 4, 5 channel segments or more each carrying the same or different types of beads, reagents, and/or biological particles that meet at a channel junction. Fluid flow in each channel segment can be controlled to control the partitioning of the different elements into droplets. Fluid can be directed flow along one or more channels or reservoirs via one or more fluid flow units. A fluid flow unit can include compressors (e.g., providing positive pressure), pumps (e.g., providing negative pressure), actuators, and the like to control flow of the fluid. Fluid can also or otherwise be controlled via applied pressure differentials, centrifugal force, electrokinetic pumping, vacuum, capillary or gravity flow, or the like.

Examples of lysis agents include bioactive reagents, such as lysis enzymes that are used for lysis of different cell types, e.g., gram positive or negative bacteria, plants, yeast, mammalian, etc., such as lysozymes, achromopeptidase, lysostaphin, labiase, kitalase, lyticase, and a variety of other lysis enzymes available from, e.g., Sigma-Aldrich, Inc. (St Louis, MO), as well as other commercially available lysis enzymes. Other lysis agents can additionally or alternatively be co-partitioned with the biological particles to cause the release of the biological particle's contents into the partitions. For example, in some cases, surfactant-based lysis solutions can be used to lyse cells (e.g., labelled engineered cells), although these can be less desirable for emulsion based systems where the surfactants can interfere with stable emulsions. In some cases, lysis solutions can include non-ionic surfactants such as, for example, Triton X-100 and Tween 20. In some cases, lysis solutions can include ionic surfactants such as, for example, sarcosyl and sodium dodecyl sulfate (SDS). Electroporation, thermal, acoustic or mechanical cellular disruption can also be used in certain cases, e.g., non-emulsion based partitioning such as encapsulation of biological particles that can be in addition to or in place of droplet partitioning, where any pore size of the encapsulate is sufficiently small to retain nucleic acid fragments of a given size, following cellular disruption.

Alternatively or in addition to the lysis agents co-partitioned with the biological particles (e.g., labelled engineered cells) described above, other reagents can also be co-partitioned with the biological particles, including, for example, DNase and RNase inactivating agents or inhibitors, such as proteinase K, chelating agents, such as EDTA, and other reagents employed in removing or otherwise reducing negative activity or impact of different cell lysate components on subsequent processing of nucleic acids. In addition, in the case of encapsulated biological particles (e.g., cell beads comprising labelled engineered cells), the biological particles can be exposed to an appropriate stimulus to release the biological particles or their contents from a co-partitioned cell bead. For example, in some cases, a chemical stimulus can be co-partitioned along with an encapsulated biological particle to allow for the degradation of the encapsulating material and release of the cell or its contents into the larger partition. In some cases, this stimulus can be the same as the stimulus described elsewhere herein for release of nucleic acid molecules (e.g., oligonucleotides) from their respective bead. In alternative aspects, this can be a different and non-overlapping stimulus, in order to allow an encapsulated biological particle to be released into a partition at a different time from the release of nucleic acid molecules into the same partition.

Additional reagents can also be co-partitioned with the biological particles (e.g., labelled engineered cells), such as endonucleases to fragment a biological particle's DNA, DNA polymerase enzymes and dNTPs used to amplify the biological particle's nucleic acid fragments and to attach the barcode molecular tags to the amplified fragments. Other enzymes can be co-partitioned, including without limitation, polymerase, transposase, ligase, proteinase K, DNAse, etc. Additional reagents can also include reverse transcriptase enzymes, including enzymes with terminal transferase activity, primers and oligonucleotides, and switch oligonucleotides (also referred to herein as “switch oligos” or “template switching oligonucleotides”) which can be used for template switching. In some cases, template switching can be used to increase the length of a cDNA. In some cases, template switching can be used to append a predefined nucleic acid sequence to the cDNA. In an example of template switching, cDNA can be generated from reverse transcription of a template, e.g., cellular mRNA, where a reverse transcriptase with terminal transferase activity can add additional nucleotides, e.g., polyC, to the cDNA in a template independent manner. Switch oligos can include sequences complementary to the additional nucleotides, e.g., polyG. The additional nucleotides (e.g., polyC) on the cDNA can hybridize to the additional nucleotides (e.g., polyG) on the switch oligo, whereby the switch oligo can be used by the reverse transcriptase as template to further extend the cDNA. Template switching oligonucleotides can include a hybridization region and a template region. The hybridization region can include any sequence capable of hybridizing to the target. In some cases, as previously described, the hybridization region includes a series of G bases to complement the overhanging C bases at the 3′ end of a cDNA molecule. The series of G bases can include 1 G base, 2 G bases, 3 G bases, 4 G bases, 5 G bases or more than 5 G bases. The template sequence can include any sequence to be incorporated into the cDNA. In some cases, the template region includes at least 1 (e.g., at least 2, 3, 4, 5 or more) tag sequences and/or functional sequences. Switch oligos can include deoxyribonucleic acids; ribonucleic acids; modified nucleic acids including 2-Aminopurine, 2,6-Diaminopurine (2-Amino-dA), inverted dT, 5-Methyl dC, 2′-deoxyInosine, Super T (5-hydroxybutynl-2′-deoxyuridine), Super G (8-aza-7-deazaguanosine), locked nucleic acids (LNAs), unlocked nucleic acids (UNAs, e.g., UNA-A, UNA-U, UNA-C, UNA-G), Iso-dG, Iso-dC, 2′ Fluoro bases (e.g., Fluoro C, Fluoro U, Fluoro A, and Fluoro G), or any combination.

In some cases, the length of a switch oligo can be at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 or 250 nucleotides or longer.

In some cases, the length of a switch oligo can be at most about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 or 250 nucleotides.

Once the contents of the cells (e.g., T cells) are released into their respective partitions, the macromolecular components (e.g., macromolecular constituents of biological particles, such as RNA, DNA, proteins, or secreted antibodies or antigen-binding fragments thereof) contained therein can be further processed within the partitions. In accordance with the methods and systems described herein, the macromolecular component contents of individual biological particles (e.g., T cells) can be provided with unique identifiers such that, upon characterization of those macromolecular components they can be attributed as having been derived from the same biological particle or particles. The ability to attribute characteristics to individual biological particles or groups of biological particles is provided by the assignment of unique identifiers specifically to an individual biological particle or groups of biological particles. Unique identifiers, e.g., in the form of nucleic acid barcodes can be assigned or associated with individual biological particles or populations of biological particles, in order to tag or label the biological particle's macromolecular components (and as a result, its characteristics) with the unique identifiers. These unique identifiers can then be used to attribute the biological particle's components and characteristics to an individual biological particle or group of biological particles.

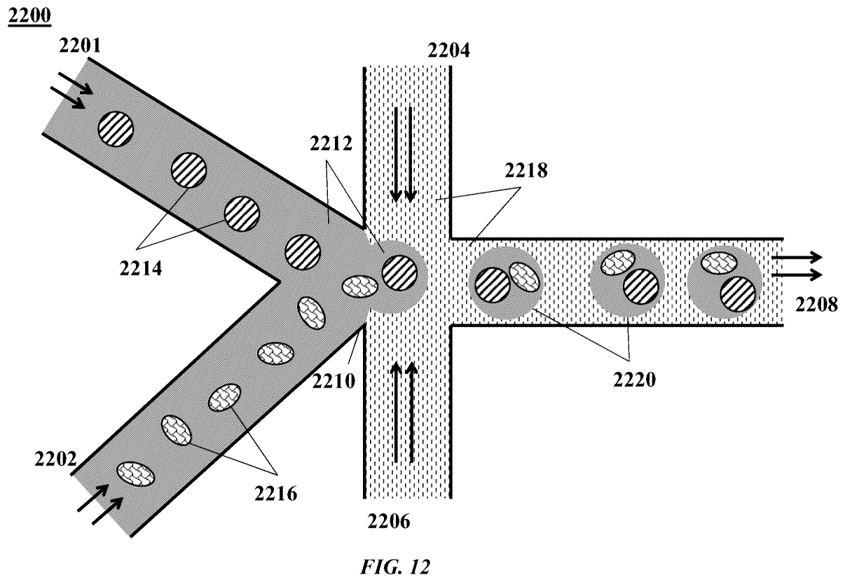

In some aspects, this is performed by co-partitioning the individual biological particle (e.g., T cells) or groups of biological particles (e.g., T cells) with the unique identifiers, such as described above (with reference to FIGS. 12 and 13). In some aspects, the unique identifiers are provided in the form of nucleic acid molecules (e.g., oligonucleotides) that include nucleic acid barcode sequences that can be attached to or otherwise associated with the nucleic acid contents of individual biological particle, or to other components of the biological particle, and particularly to fragments of those nucleic acids. The nucleic acid molecules are partitioned such that as between nucleic acid molecules in a given partition, the nucleic acid barcode sequences contained therein are the same, but as between different partitions, the nucleic acid molecule can, and do have differing barcode sequences, or at least represent a large number of different barcode sequences across all of the partitions in a given analysis. In some aspects, only one nucleic acid barcode sequence can be associated with a given partition, although in some cases, two or more different barcode sequences can be present.

The nucleic acid barcode sequences can include from about 6 to about 20 or more nucleotides within the sequence of the nucleic acid molecules (e.g., oligonucleotides). The nucleic acid barcode sequences can include from about 6 to about 20, 30, 40, 50, 60, 70, 80, 90, 100 or more nucleotides. In some cases, the length of a barcode sequence can be about 6, 7, 8, 9, 10, 11, 12, 13, 14, 15, 16, 17, 18, 19, 20 nucleotides or longer. In some cases, the length of a barcode sequence can be at least about 6, 7, 8, 9, 10, 11, 12, 13, 14, 15, 16, 17, 18, 19, 20 nucleotides or longer. In some cases, the length of a barcode sequence can be at most about 6, 7, 8, 9, 10, 11, 12, 13, 14, 15, 16, 17, 18, 19, 20 nucleotides or shorter. These nucleotides can be completely contiguous, i.e., in a single stretch of adjacent nucleotides, or they can be separated into two or more separate subsequences that are separated by 1 or more nucleotides. In some cases, separated barcode subsequences can be from about 4 to about 16 nucleotides in length. In some cases, the barcode subsequence can be about 4, 5, 6, 7, 8, 9, 10, 11, 12, 13, 14, 15, 16 nucleotides or longer. In some cases, the barcode subsequence can be at least about 4, 5, 6, 7, 8, 9, 10, 11, 12, 13, 14, 15, 16 nucleotides or longer. In some cases, the barcode subsequence can be at most about 4, 5, 6, 7, 8, 9, 10, 11, 12, 13, 14, 15, 16 nucleotides or shorter.

The co-partitioned nucleic acid molecules can also include other functional sequences useful in the processing of the nucleic acids from the co-partitioned biological particles (e.g., labelled T cells). These sequences include, e.g., targeted or random/universal amplification primer sequences for amplifying the genomic DNA from the individual biological particles within the partitions while attaching the associated barcode sequences, sequencing primers or primer recognition sites, hybridization or probing sequences, e.g., for identification of presence of the sequences or for pulling down barcoded nucleic acids, or any of a number of other potential functional sequences. Other mechanisms of co-partitioning oligonucleotides can also be employed, including, e.g., coalescence of two or more droplets, where one droplet contains oligonucleotides, or microdispensing of oligonucleotides into partitions, e.g., droplets within microfluidic systems.

In an example, beads are provided that each include large numbers of the above described nucleic acid barcode molecules (e.g., barcoded oligonucleotides) releasably attached to the beads, where all of the nucleic acid molecules attached to a particular bead will include the same nucleic acid barcode sequence, but where a large number of diverse barcode sequences are represented across the population of beads used. In some embodiments, hydrogel beads, e.g., including polyacrylamide polymer matrices, are used as a solid support and delivery vehicle for the nucleic acid molecules into the partitions, as they are capable of carrying large numbers of nucleic acid molecules, and can be configured to release those nucleic acid molecules upon exposure to a particular stimulus, as described elsewhere herein. In some cases, the population of beads provides a diverse barcode sequence library that includes at least about 1,000 different barcode sequences, at least about 5,000 different barcode sequences, at least about 10,000 different barcode sequences, at least about 50,000 different barcode sequences, at least about 100,000 different barcode sequences, at least about 1,000,000 different barcode sequences, at least about 5,000,000 different barcode sequences, or at least about 10,000,000 different barcode sequences, or more. Additionally, each bead can be provided with large numbers of nucleic acid (e.g., oligonucleotide) molecules attached. In particular, the number of molecules of nucleic acid molecules including the barcode sequence on an individual bead can be at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some cases at least about 1 billion nucleic acid molecules, or more. Nucleic acid molecules of a given bead can include identical (or common) barcode sequences, different barcode sequences, or a combination of both. Nucleic acid molecules of a given bead can include multiple sets of nucleic acid molecules. Nucleic acid molecules of a given set can include identical barcode sequences. The identical barcode sequences can be different from barcode sequences of nucleic acid molecules of another set.

Moreover, when the population of beads is partitioned, the resulting population of partitions can also include a diverse barcode library that includes at least about 1,000 different barcode sequences, at least about 5,000 different barcode sequences, at least about 10,000 different barcode sequences, at least at least about 50,000 different barcode sequences, at least about 100,000 different barcode sequences, at least about 1,000,000 different barcode sequences, at least about 5,000,000 different barcode sequences, or at least about 10,000,000 different barcode sequences. Additionally, each partition of the population can include at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some cases at least about 1 billion nucleic acid molecules.

In some cases, it may be desirable to incorporate multiple different barcodes within a given partition, either attached to a single or multiple beads within the partition. For example, in some cases, a mixed, but known set of barcode sequences can provide greater assurance of identification in the subsequent processing, e.g., by providing a stronger address or attribution of the barcodes to a given partition, as a duplicate or independent confirmation of the output from a given partition.

The nucleic acid molecules (e.g., oligonucleotides) are releasable from the beads upon the application of a particular stimulus to the beads. In some cases, the stimulus can be a photo-stimulus, e.g., through cleavage of a photo-labile linkage that releases the nucleic acid molecules. In other cases, a thermal stimulus can be used, where elevation of the temperature of the beads environment will result in cleavage of a linkage or other release of the nucleic acid molecules from the beads. In still other cases, a chemical stimulus can be used that cleaves a linkage of the nucleic acid molecules to the beads, or otherwise results in release of the nucleic acid molecules from the beads. In one case, such compositions include the polyacrylamide matrices described above for encapsulation of biological particles, and can be degraded for release of the attached nucleic acid molecules through exposure to a reducing agent, such as DTT.

### Systems and Methods for Controlled Partitioning

In some aspects, provided are systems and methods for controlled partitioning. Droplet size can be controlled by adjusting certain geometric features in channel architecture (e.g., microfluidics channel architecture). For example, an expansion angle, width, and/or length of a channel can be adjusted to control droplet size.

FIG. 12 shows an example of a microfluidic channel structure 1400 for delivering barcode carrying beads to droplets. The channel structure 1400 can include channel segments 1401, 1416, 1404, 1406 and 1408 communicating at a channel junction 1410. In operation, the channel segment 1401 may transport an aqueous fluid 1412 that includes a plurality of beads 1414 (e.g., with nucleic acid molecules, oligonucleotides, molecular tags) along the channel segment 1401 into junction 1410. The plurality of beads 1414 may be sourced from a suspension of beads. For example, the channel segment 1401 may be connected to a reservoir comprising an aqueous suspension of beads 1414. The channel segment 1402 may transport the aqueous fluid 1412 that includes a plurality of biological particles 1416 along the channel segment 1402 into junction 1410. The plurality of biological particles 1416 may be sourced from a suspension of biological particles. For example, the channel segment 1402 may be connected to a reservoir comprising an aqueous suspension of biological particles 1416. In some instances, the aqueous fluid 1412 in either the first channel segment 1401 or the second channel segment 1402, or in both segments, can include one or more reagents, as further described below. A second fluid 1418 that is immiscible with the aqueous fluid 1412 (e.g., oil) can be delivered to the junction 1410 from each of channel segments 1404 and 1406. Upon meeting of the aqueous fluid 1412 from each of channel segments 1401 and 1402 and the second fluid 1418 from each of channel segments 1404 and 1406 at the channel junction 1410, the aqueous fluid 1412 can be partitioned as discrete droplets 1420 in the second fluid 1418 and flow away from the junction 1410 along channel segment 1408. The channel segment 1408 may deliver the discrete droplets to an outlet reservoir fluidly coupled to the channel segment 1408, where they may be harvested. As an alternative, the channel segments 1401 and 1402 may meet at another junction upstream of the junction 1410. At such junction, beads and biological particles may form a mixture that is directed along another channel to the junction 1410 to yield droplets 1420. The mixture may provide the beads and biological particles in an alternating fashion, such that, for example, a droplet comprises a single bead and a single biological particle.

FIG. 5 shows an example of a microfluidic channel structure for the controlled partitioning of beads into discrete droplets. A channel structure 1300 can include a channel segment 1302 communicating at a channel junction 1306 (or intersection) with a reservoir 1304. The reservoir 1304 can be a chamber. Any reference to “reservoir,” as used herein, can also refer to a “chamber.” In operation, an aqueous fluid 1308 that includes suspended beads 1312 can be transported along the channel segment 1302 into the junction 1306 to meet a second fluid 1310 that is immiscible with the aqueous fluid 1308 in the reservoir 1304 to create droplets 1316, 1318 of the aqueous fluid 1308 flowing into the reservoir 1304. At the junction 1306 where the aqueous fluid 1308 and the second fluid 1310 meet, droplets can form based on factors such as the hydrodynamic forces at the junction 1306, flow rates of the two fluids 1308, 1310, fluid properties, and certain geometric parameters (e.g., w, h₀, α, etc.) of the channel structure 1300. A plurality of droplets can be collected in the reservoir 1304 by continuously injecting the aqueous fluid 1308 from the channel segment 1302 through the junction 1306.

A discrete droplet generated can include a bead (e.g., as in occupied droplets 1316). Alternatively, a discrete droplet generated can include more than one bead. Alternatively, a discrete droplet generated cannot include any beads (e.g., as in unoccupied droplet 1318). In some instances, a discrete droplet generated can contain one or more biological particles, as described elsewhere herein. In some instances, a discrete droplet generated can include one or more reagents, as described elsewhere herein.

In some instances, the aqueous fluid 1308 can have a substantially uniform concentration or frequency of beads 1312. The beads 1312 can be introduced into the channel segment 1302 from a separate channel (not shown in FIG. 5). The frequency of beads 1312 in the channel segment 1302 can be controlled by controlling the frequency in which the beads 1312 are introduced into the channel segment 1302 and/or the relative flow rates of the fluids in the channel segment 1302 and the separate channel. In some instances, the beads can be introduced into the channel segment 1302 from a plurality of different channels, and the frequency controlled accordingly.

In some instances, the aqueous fluid 1308 in the channel segment 1302 can include biological particles (e.g., described with reference to FIG. 4). In some instances, the aqueous fluid 1308 can have a substantially uniform concentration or frequency of biological particles. As with the beads, the biological particles (e.g., labelled engineered cells) can be introduced into the channel segment 1302 from a separate channel. The frequency or concentration of the biological particles in the aqueous fluid 1308 in the channel segment 1302 can be controlled by controlling the frequency in which the biological particles are introduced into the channel segment 1302 and/or the relative flow rates of the fluids in the channel segment 1302 and the separate channel. In some instances, the biological particles can be introduced into the channel segment 1302 from a plurality of different channels, and the frequency controlled accordingly. In some instances, a first separate channel can introduce beads and a second separate channel can introduce biological particles into the channel segment 1302. The first separate channel introducing the beads can be upstream or downstream of the second separate channel introducing the biological particles.

The second fluid 1310 can include an oil, such as a fluorinated oil, that includes a fluorosurfactant for stabilizing the resulting droplets, for example, inhibiting subsequent coalescence of the resulting droplets.

In some instances, the second fluid 1310 cannot be subjected to and/or directed to any flow in or out of the reservoir 1304. For example, the second fluid 1310 can be substantially stationary in the reservoir 1304. In some instances, the second fluid 1310 can be subjected to flow within the reservoir 1304, but not in or out of the reservoir 1304, such as via application of pressure to the reservoir 1304 and/or as affected by the incoming flow of the aqueous fluid 1308 at the junction 1306. Alternatively, the second fluid 1310 can be subjected and/or directed to flow in or out of the reservoir 1304. For example, the reservoir 1304 can be a channel directing the second fluid 1310 from upstream to downstream, transporting the generated droplets.

The channel structure 1300 at or near the junction 1306 can have certain geometric features that at least partly determine the sizes of the droplets formed by the channel structure 1300. The channel segment 1302 can have a height, h₀ and width, w, at or near the junction 1306. By way of example, the channel segment 1302 can include a rectangular cross-section that leads to a reservoir 1304 having a wider cross-section (such as in width or diameter). Alternatively, the cross-section of the channel segment 1302 can be other shapes, such as a circular shape, trapezoidal shape, polygonal shape, or any other shapes. The top and bottom walls of the reservoir 1304 at or near the junction 1306 can be inclined at an expansion angle, a. The expansion angle, a, allows the tongue (portion of the aqueous fluid 1308 leaving channel segment 1302 at junction 1306 and entering the reservoir 1304 before droplet formation) to increase in depth and facilitate decrease in curvature of the intermediately formed droplet. Droplet size can decrease with increasing expansion angle. The resulting droplet radius, Ra, can be predicted by the following equation for the aforementioned geometric parameters of h₀, w, and α:

$${R}_{d} \approx 0.44 ( 1 + 2.2 \sqrt{tan α} \frac{w}{{h}_{0}} ) \frac{{h}_{0}}{\sqrt{tan α}}$$

By way of example, for a channel structure with w=21 μm, h=21 μm, and α=3º, the predicted droplet size is 121 μm. In another example, for a channel structure with w=25 μm, h=25 μm, and α=5°, the predicted droplet size is 123 μm. In another example, for a channel structure with w=28 μm, h=28 μm, and α=7º, the predicted droplet size is 124 μm.

In some instances, the expansion angle, a, can be between a range of from about 0.5° to about 4°, from about 0.1° to about 10°, or from about 0° to about 90°. For example, the expansion angle can be at least about 0.01°, 0.1º, 0.2°, 0.3°, 0.4°, 0.5°, 0.6°, 0.7º, 0.8°, 0.9°, 1º, 2°, 3º, 4º, 5°, 6º, 7º, 8°, 9º, 10°, 15°, 20°, 25°, 30°, 35°, 40°, 45°, 50°, 55°, 60°, 65°, 70°, 75°, 80°, 85°, or higher. In some instances, the expansion angle can be at most about 89º, 88°, 87º, 86°, 85°, 84°, 83º, 82°, 81º, 80°, 75°, 70°, 65°, 60°, 55°, 50°, 45°, 40°, 35°, 30°, 25°, 20°, 15°, 10°, 9º, 8°, 7º, 6°, 5°, 4º, 3º, 2º, 1º, 0.1º, 0.01°, or less. In some instances, the width, w, can be between a range of from about 100 micrometers (μm) to about 500 μm. In some instances, the width, w, can be between a range of from about 10 μm to about 200 μm. Alternatively, the width can be less than about 10 μm. Alternatively, the width can be greater than about 500 μm. In some instances, the flow rate of the aqueous fluid 1308 entering the junction 1306 can be between about 0.04 microliters (μL)/minute (min) and about 40 μL/min. In some instances, the flow rate of the aqueous fluid 1308 entering the junction 1306 can be between about 0.01 microliters (μL)/minute (min) and about 100 L/min. Alternatively, the flow rate of the aqueous fluid 1308 entering the junction 1306 can be less than about 0.01 L/min. Alternatively, the flow rate of the aqueous fluid 1308 entering the junction 1306 can be greater than about 40 μL/min, such as 45 μL/min, 50 μL/min, 55 μL/min, 60 μL/min, 65 μL/min, 70 μL/min, 75 μL/min, 80 μL/min, 85 μL/min, 90 L/min, 95 μL/min, 100 μL/min, 110 μL/min, 120 μL/min, 130 μL/min, 140 μL/min, 150 μL/min, or greater. At lower flow rates, such as flow rates of about less than or equal to 10 microliters/minute, the droplet radius cannot be dependent on the flow rate of the aqueous fluid 1308 entering the junction 1306.

In some instances, at least about 50% of the droplets generated can have uniform size. In some instances, at least about 55%, 60%, 65%, 70%, 75%, 80%, 85%, 90%, 95%, 96%, 97%, 98%, 99%, or greater of the droplets generated can have uniform size. Alternatively, less than about 50% of the droplets generated can have uniform size.

The throughput of droplet generation can be increased by increasing the points of generation, such as increasing the number of junctions (e.g., junction 1306) between aqueous fluid 1308 channel segments (e.g., channel segment 1302) and the reservoir 1304. Alternatively or in addition, the throughput of droplet generation can be increased by increasing the flow rate of the aqueous fluid 1308 in the channel segment 1302.

The methods and systems described herein can be used to greatly increase the efficiency of single cell applications and/or other applications receiving droplet-based input.

Subsequent operations that can be performed can include generation of amplification products, purification (e.g., via solid phase reversible immobilization (SPRI)), further processing (e.g., shearing, ligation of functional sequences, and subsequent amplification (e.g., via PCR)). These operations can occur in bulk (e.g., outside the partition). In the case where a partition is a droplet in an emulsion, the emulsion can be broken and the contents of the droplet pooled for additional operations. Additional reagents that can be co-partitioned along with the barcode bearing bead can include oligonucleotides to block ribosomal RNA (rRNA) and nucleases to digest genomic DNA from cells. Alternatively, rRNA removal agents can be applied during additional processing operations. The configuration of the constructs generated by such a method can help minimize (or avoid) sequencing of the poly-T sequence during sequencing and/or sequence the 5′ end of a polynucleotide sequence. The amplification products, for example, first amplification products and/or second amplification products, can be subject to sequencing for sequence analysis. In some cases, amplification can be performed using the Partial Hairpin Amplification for Sequencing (PHASE) method.

A variety of applications require the evaluation of the presence and quantification of different biological particle or organism types within a population of biological particles, including, for example, microbiome analysis and characterization, environmental testing, food safety testing, epidemiological analysis, e.g., in tracing contamination or the like.

Partitions including a barcode bead (e.g., a gel bead) associated with barcode molecules and a bead encapsulating cellular constituents (e.g., a cell bead) such as cellular nucleic acids can be useful in constituent analysis as is described in U.S. Patent Publication No. 2018/0216162.

### Sample and Cell Processing

A sample can be derived from any useful source including any subject, such as a human subject. A sample can include material (e.g., one or more cells) from one or more different sources, such as one or more different subjects. Multiple samples, such as multiple samples from a single subject (e.g., multiple samples obtained in the same or different manners from the same or different bodily locations, and/or obtained at the same or different times (e.g., seconds, minutes, hours, days, weeks, months, or years apparat)), or multiple samples from different subjects, can be obtained for analysis as described herein. For example, a first sample can be obtained from a subject at a first time and a second sample can be obtained from the subject at a second time later than the first time. The first time can be before a subject undergoes a treatment regimen or procedure (e.g., to address a disease or condition), and the second time can be during or after the subject undergoes the treatment regimen or procedure. In another example, a first sample can be obtained from a first bodily location or system of a subject (e.g., using a first collection technique) and a second sample can be obtained from a second bodily location or system of the subject (e.g., using a second collection technique), which second bodily location or system can be different than the first bodily location or system. In another example, multiple samples can be obtained from a subject at a same time from the same or different bodily locations. Different samples, such as different samples collected from different bodily locations of a same subject, at different times, from multiple different subjects, and/or using different collection techniques, can undergo the same or different processing (e.g., as described herein). For example, a first sample can undergo a first processing protocol and a second sample can undergo a second processing protocol.

A sample can be a biological sample, such as a cell sample (e.g., as described herein). A sample can include one or more biological particles, such as one or more cells and/or cellular constituents, such as one or more cell nuclei. For example, a sample can include a plurality of cells and/or cellular constituents. Components (e.g., cells or cellular constituents, such as cell nuclei) of a sample can be of a single type or a plurality of different types. For example, cells of a sample can include one or more different types of blood cells.

A biological sample can include a plurality of cells having different dimensions and features. In some cases, processing of the biological sample, such as cell separation and sorting (e.g., as described herein), can affect the distribution of dimensions and cellular features included in the sample by depleting cells having certain features and dimensions and/or isolating cells having certain features and dimensions.

A sample may undergo one or more processes in preparation for analysis (e.g., as described herein), including, but not limited to, filtration, selective precipitation, purification, centrifugation, permeabilization, isolation, agitation, heating, and/or other processes. For example, a sample may be filtered to remove a contaminant or other materials. In an example, a filtration process can include the use of microfluidics (e.g., to separate biological particles of different sizes, types, charges, or other features).

In an example, a sample including one or more cells can be processed to separate the one or more cells from other materials in the sample (e.g., using centrifugation and/or another process). In some cases, cells and/or cellular constituents of a sample can be processed to separate and/or sort groups of cells and/or cellular constituents, such as to separate and/or sort cells and/or cellular constituents of different types. Examples of cell separation include, but are not limited to, separation of white blood cells or immune cells from other blood cells and components, separation of circulating tumor cells from blood, and separation of bacteria from bodily cells and/or environmental materials. A separation process can include a positive selection process (e.g., targeting of a cell type of interest for retention for subsequent downstream analysis, such as by use of a monoclonal antibody that targets a surface marker of the cell type of interest), a negative selection process (e.g., removal of one or more cell types and retention of one or more other cell types of interest), and/or a depletion process (e.g., removal of a single cell type from a sample, such as removal of red blood cells from peripheral blood mononuclear cells).

Separation of one or more different types of cells can include, for example, centrifugation, filtration, microfluidic-based sorting, flow cytometry, fluorescence-activated cell sorting (FACS), magnetic-activated cell sorting (MACS), buoyancy-activated cell sorting (BACS), or any other useful method. For example, a flow cytometry method can be used to detect cells and/or cellular constituents based on a parameter such as a size, morphology, or protein expression. Flow cytometry-based cell sorting can include injecting a sample into a sheath fluid that conveys the cells and/or cellular constituents of the sample into a measurement region one at a time. In the measurement region, a light source such as a laser can interrogate the cells and/or cellular constituents and scattered light and/or fluorescence can be detected and converted into digital signals. A nozzle system (e.g., a vibrating nozzle system) can be used to generate droplets (e.g., aqueous droplets) including individual cells and/or cellular constituents. Droplets including cells and/or cellular constituents of interest (e.g., as determined via optical detection) can be labeled with an electric charge (e.g., using an electrical charging ring), which charge can be used to separate such droplets from droplets including other cells and/or cellular constituents. For example, FACS can include labeling cells and/or cellular constituents with fluorescent markers (e.g., using internal and/or external biomarkers). Cells and/or cellular constituents can then be measured and identified one by one and sorted based on the emitted fluorescence of the marker or absence thereof. MACS can use micro- or nano-scale magnetic particles to bind to cells and/or cellular constituents (e.g., via an antibody interaction with cell surface markers) to facilitate magnetic isolation of cells and/or cellular constituents of interest from other components of a sample (e.g., using a column-based analysis). BACS can use microbubbles (e.g., glass microbubbles) labeled with antibodies to target cells of interest. Cells and/or cellular components coupled to microbubbles can float to a surface of a solution, thereby separating target cells and/or cellular components from other components of a sample. Cell separation techniques can be used to enrich for populations of cells of interest (e.g., prior to partitioning, as described herein). For example, a sample including a plurality of cells including a plurality of cells of a given type can be subjected to a positive separation process. The plurality of cells of the given type can be labeled with a fluorescent marker (e.g., based on an expressed cell surface marker or another marker) and subjected to a FACS process to separate these cells from other cells of the plurality of cells. The selected cells can then be subjected to subsequent partition-based analysis (e.g., as described herein) or other downstream analysis. The fluorescent marker can be removed prior to such analysis or can be retained. The fluorescent marker can include an identifying feature, such as a nucleic acid barcode sequence and/or unique molecular identifier.

In another example, a first sample including a first plurality of cells including a first plurality of cells of a given type (e.g., immune cells expressing a particular marker or combination of markers) and a second sample including a second plurality of cells including a second plurality of cells of the given type can be subjected to a positive separation process. The first and second samples can be collected from the same or different subjects, at the same or different types, from the same or different bodily locations or systems, using the same or different collection techniques. For example, the first sample can be from a first subject and the second sample can be from a second subject different than the first subject. The first plurality of cells of the first sample can be provided a first plurality of fluorescent markers configured to label the first plurality of cells of the given type. The second plurality of cells of the second sample can be provided a second plurality of fluorescent markers configured to label the second plurality of cells of the given type. The first plurality of fluorescent markers can include a first identifying feature, such as a first barcode, while the second plurality of fluorescent markers can include a second identifying feature, such as a second barcode, that is different than the first identifying feature. The first plurality of fluorescent markers and the second plurality of fluorescent markers can fluoresce at the same intensities and over the same range of wavelengths upon excitation with a same excitation source (e.g., light source, such as a laser). The first and second samples can then be combined and subjected to a FACS process to separate cells of the given type from other cells based on the first plurality of fluorescent markers labeling the first plurality of cells of the given type and the second plurality of fluorescent markers labeling the second plurality of cells of the given typc. Alternatively, the first and second samples can undergo separate FACS processes and the positively selected cells of the given type from the first sample and the positively selected cells of the given type from the second sample can then be combined for subsequent analysis. The encoded identifying features of the different fluorescent markers can be used to identify cells originating from the first sample and cells originating from the second sample. For example, the first and second identifying features can be configured to interact (e.g., in partitions, as described herein) with nucleic acid barcode molecules (e.g., as described herein) to generate barcoded nucleic acid products detectable using, e.g., nucleic acid sequencing.

FIG. 18 schematically shows an example workflow for processing nucleic acid molecules within a sample. A substrate 1800 including a plurality of microwells 1802 can be provided. A sample 1806 which can include a cell, cell bead, cellular components or analytes (e.g., proteins and/or nucleic acid molecules) can be co-partitioned, in a plurality of microwells 1802, with a plurality of beads 1804 including nucleic acid barcode molecules. During a partitioning process, the sample 1806 can be processed within the partition. For instance, in the case of live cells, the cell can be subjected to conditions sufficient to lyse the cells and release the analytes contained therein. In process 1820, the bead 1804 can be further processed. By way of example, processes 1820a and 1820b schematically illustrate different workflows, depending on the properties of the bead 1804.

In 1820a, the bead includes nucleic acid barcode molecules that are attached thereto, and sample nucleic acid molecules (e.g., RNA, DNA) can attach, e.g., via hybridization of ligation, to the nucleic acid barcode molecules. Such attachment can occur on the bead. In process 1830, the beads 1804 from multiple wells 1802 can be collected and pooled. Further processing can be performed in process 1840. For example, one or more nucleic acid reactions can be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences can be appended to each end of the nucleic acid molecule. In process 1850, further characterization, such as sequencing can be performed to generate sequencing reads. The sequencing reads can yield information on individual cells or populations of cells, which can be represented visually or graphically, e.g., in a plot.

In 1820b, the bead includes nucleic acid barcode molecules that are releasably attached thereto, as described below. The bead can degrade or otherwise release the nucleic acid barcode molecules into the well 1802; the nucleic acid barcode molecules can then be used to barcode nucleic acid molecules within the well 1802. Further processing can be performed either inside the partition or outside the partition. For example, one or more nucleic acid reactions can be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences can be appended to each end of the nucleic acid molecule. In process 1850, further characterization, such as sequencing can be performed to generate sequencing reads. The sequencing reads can yield information on individual cells or populations of cells, which can be represented visually or graphically, e.g., in a plot.

### Targeting Processes

The methods provided herein may comprise the use of a targeting process to, e.g., enrich selected nucleic acid molecules within a sample.

An exemplary target enrichment method may comprise providing a plurality of barcoded nucleic acid molecules and hybridizing barcoded nucleic acid molecules comprising targeted regions of interest to oligonucleotide probes (“baits”) which are complementary to the targeted regions of interest (or to regions near or adjacent to the targeted regions of interest). Baits may be attached to a capture molecule, including without limitation a biotin molecule. The capture molecule (e.g., biotin) can be used to selectively pull down the targeted regions of interest (for example, with magnetic streptavidin beads) to thereby enrich the resultant population of barcoded nucleic acid molecules for those containing the targeted regions of interest.

### Multiplexing Methods

In some embodiments of the disclosure, steps (a) and (b) of the methods described herein are performed in multiplex format. For example, in some embodiments, step (a) of the methods disclosed herein can include individually partitioning additional single cells (e.g., T cells) of the plurality of cells in additional partitions of the plurality of partitions, and step (b) can further include determining all or a part of the nucleic acid sequences encoding antibodies or antigen-binding fragments thereof produced by the additional cells (e.g., T cells).

Accordingly, in some embodiments, the present disclosure provides methods and systems for multiplexing, and otherwise increasing throughput of samples for analysis. For example, a single or integrated process workflow may permit the processing, identification, and/or analysis of more or multiple analytes, more or multiple types of analytes, and/or more or multiple types of analyte characterizations. For example, in the methods and systems described herein, one or more labelling agents capable of binding to or otherwise coupling to one or more cells or cell features can be used to characterize cells and/or cell features. In some instances, cell features include cell surface features. Cell surface features can include, but are not limited to, a receptor, an antigen or antigen fragment (e.g., an antigen or antigen fragment that binds to an antigen-binding molecule located on a cell surface), a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, a B-cell receptor, a chimeric antigen receptor, a gap junction, an adherens junction, or any combination thereof. In some instances, cell features can include intracellular analytes, such as proteins, protein modifications (e.g., phosphorylation status or other post-translational modifications), nuclear proteins, nuclear membrane proteins, or any combination thereof. A labelling agent can include, but is not limited to, a protein, a peptide, an antibody (or an epitope binding fragment thereof), an antigen, an antigen fragment, a lipophilic moiety (such as cholesterol), a cell surface receptor binding molecule, a receptor ligand, a small molecule, a bi-specific antibody, a B-cell receptor engager, a pro-body, an aptamer, a monobody, an affimer, a Darpin, and a protein scaffold, or any combination thereof. The labelling agents can include (e.g., are attached to) a reporter oligonucleotide that is indicative of the cell surface feature to which the binding group binds. For example, the reporter oligonucleotide can include a barcode sequence that permits identification of the labelling agent. For example, a labelling agent that is specific to one type of cell feature (e.g., a first cell surface feature) can have a first reporter oligonucleotide coupled thereto, while a labelling agent that is specific to a different cell feature (e.g., a second cell surface feature) can have a different reporter oligonucleotide coupled thereto. For a description of exemplary labelling agents, reporter oligonucleotides, and methods of use, see, e.g., U.S. Pat. No. 10,550,429; U.S. Pat. Pub. 20190177800; and U.S. Pat. Pub. 20190367969.

In a particular example, a library of potential cell feature labelling agents can be provided, where the respective cell feature labelling agents are associated with nucleic acid reporter molecules, such that a different reporter oligonucleotide sequence is associated with each labelling agent capable of binding to a specific cell feature. In some embodiments, the cell feature labelling agents comprise a target antigen and a fragment of the target antigen, as disclosed herein. In some embodiments, the cell feature labelling agents comprise a plurality of non-overlapping fragments of a target antigen. In other aspects, different members of the library can be characterized by the presence of a different oligonucleotide sequence label. For example, an antibody capable of binding to a target protein can have associated with it a first reporter oligonucleotide sequence, while an antibody, (which may be the same antibody), capable of binding to a fragment or fragments of the target antigen can have a different, (or additional if the same antibody), reporter oligonucleotide sequence(s) associated with it. The presence of the particular oligonucleotide sequence(s) can be indicative of the presence of a particular antibody or cell feature which can be recognized or bound by the particular antibody.

Labelling agents capable of binding to or otherwise coupling to one or more cells can be used to characterize a cell as belonging to a particular set of cells. For example, labellinging agents can be used to label a sample of cells, e.g., to provide a sample index. For other example, labelling agents can be used to label a group of cells belonging to a particular experimental condition. In this way, a group of cells can be labeled as different from another group of cells. In an example, a first group of cells can originate from a first sample and a second group of cells can originate from a second sample. Labelling agents can allow the first group and second group to have a different labeling agent (or reporter oligonucleotide associated with the labeling agent). This can, for example, facilitate multiplexing, where cells of the first group and cells of the second group can be labeled separately and then pooled together for downstream analysis. The downstream detection of a label can indicate analytes as belonging to a particular group.

For example, a reporter oligonucleotide can be linked to an antibody or an epitope binding fragment thereof, and labeling a cell can include subjecting the antibody-linked barcode molecule or the epitope binding fragment-linked barcode molecule to conditions suitable for binding the antibody to a molecule present on a surface of the cell. The binding affinity between the antibody or the epitope binding fragment thereof and the molecule present on the surface can be within a desired range to ensure that the antibody or the epitope binding fragment thereof remains bound to the molecule. For example, the binding affinity can be within a desired range to ensure that the antibody or the epitope binding fragment thereof remains bound to the molecule during various sample processing steps, such as partitioning and/or nucleic acid amplification or extension. A dissociation constant (Kd) between the antibody or an epitope binding fragment thereof and the molecule to which it binds can be less than about 100 μM, 90 μM, 80 μM, 70 μM, 60 μM, 50 μM, 40 M, 30 μM, 20 μM, 10 μM, 9 μM, 8 μM, 7 μM, 6 μM, 5 μM, 4 μM, 3 μM, 2 μM, 1 μM, 900 μM, 800 μM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1 nM, 900 pM, 800 pM, 700 pM, 600 pM, 500 pM, 400 pM, 300 pM, 200 pM, 100 pM, 90 pM, 80 pM, 70 pM, 60 pM, 50 pM, 40 pM, 30 pM, 20 pM, 10 pM, 9 pM, 8 pM, 7 pM, 6 pM, 5 pM, 4 pM, 3 pM, 2 pM, or 1 pM. For example, the dissociation constant can be less than about 10 μM. In some embodiments, the antibody or epitope binding fragment thereof has a desired off rate (koff), such that the antibody or antigen binding fragment thereof remains bound to the target antigen or antigen fragment during various sample processing steps.

In another example, a reporter oligonucleotide can be coupled to a cell-penetrating peptide (CPP), and labeling cells can include delivering the CPP coupled reporter oligonucleotide into a biological particle. Labeling biological particles can include delivering the CPP conjugated oligonucleotide into a cell and/or cell bead by the cell-penetrating peptide. A CPP that can be used in the methods provided herein can include at least one non-functional cysteine residue, which can be either free or derivatized to form a disulfide link with an oligonucleotide that has been modified for such linkage. Non-limiting examples of CPPs that can be used in embodiments herein include penetratin, transportan, plsl, TAT (48-60), pVEC, MTS, and MAP. Cell-penetrating peptides useful in the methods provided herein can have the capability of inducing cell penetration for at least about 30%, 40%, 50%, 60%, 70%, 80%, 90%, 95%, 96%, 97%, 98%, 99%, or 100% of cells of a cell population. The CPP can be an arginine-rich peptide transporter. The CPP can be Penetratin or the Tat peptide. In another example, a reporter oligonucleotide can be coupled to a fluorophore or dye, and labeling cells can include subjecting the fluorophore-linked barcode molecule to conditions suitable for binding the fluorophore to the surface of the cell. In some instances, fluorophores can interact strongly with lipid bilayers and labeling cells can include subjecting the fluorophore-linked barcode molecule to conditions such that the fluorophore binds to or is inserted into a membrane of the cell. In some cases, the fluorophore is a water-soluble, organic fluorophore. In some instances, the fluorophore is Alexa 532 maleimide, tetramethylrhodamine-5-maleimide (TMR maleimide), BODIPY-TMR maleimide, Sulfo-Cy3 maleimide, Alexa 546 carboxylic acid/succinimidyl ester, Atto 550 maleimide, Cy3 carboxylic acid/succinimidyl ester, Cy3B carboxylic acid/succinimidyl ester, Atto 565 biotin, Sulforhodamine B, Alexa 594 maleimide, Texas Red maleimide, Alexa 633 maleimide, Abberior STAR 635P azide, Atto 647N maleimide, Atto 647 SE, or Sulfo-Cy5 malcimide. Sec, e.g., Hughes L D, et al. PLOS One. 2014 Feb. 4; 9 (2): e87649 for a description of organic fluorophores.

A reporter oligonucleotide can be coupled to a lipophilic molecule, and labeling cells can include delivering the nucleic acid barcode molecule to a membrane of a cell or a nuclear membrane by the lipophilic molecule. Lipophilic molecules can associate with and/or insert into lipid membranes such as cell membranes and nuclear membranes. In some cases, the insertion can be reversible. In some cases, the association between the lipophilic molecule and the cell or nuclear membrane can be such that the membrane retains the lipophilic molecule (e.g., and associated components, such as nucleic acid barcode molecules, thereof) during subsequent processing (e.g., partitioning, cell permeabilization, amplification, pooling, etc.). The reporter nucleotide can enter into the intracellular space and/or a cell nucleus. In some embodiments, a reporter oligonucleotide coupled to a lipophilic molecule will remain associated with and/or inserted into lipid membrane (as described herein) via the lipophilic molecule until lysis of the cell occurs, e.g., inside a partition. Exemplary embodiments of lipophilic molecules coupled to reporter oligonucleotides are described in PCT/US2018/064600.

A reporter oligonucleotide can be part of a nucleic acid molecule including any number of functional sequences, as described elsewhere herein, such as a target capture sequence, a random primer sequence, and the like, and coupled to another nucleic acid molecule that is, or is derived from, the analyte.

Prior to partitioning, the cells can be incubated with the library of labelling agents, that can be labelling agents to a broad panel of different cell features, e.g., receptors, proteins, etc., and which include their associated reporter oligonucleotides. Unbound labelling agents can be washed from the cells, and the cells can then be co-partitioned (e.g., into droplets or wells) along with partition-specific barcode oligonucleotides (e.g., attached to a support, such as a bead or gel bead) as described elsewhere herein. As a result, the partitions can include the cell or cells, as well as the bound labelling agents and their known, associated reporter oligonucleotides.

In other instances, e.g., to facilitate sample multiplexing, a labelling agent that is specific to a particular cell feature can have a first plurality of the labelling agent (e.g., an antibody or lipophilic moiety) coupled to a first reporter oligonucleotide and a second plurality of the labelling agent coupled to a second reporter oligonucleotide. For example, the first plurality of the labeling agent and second plurality of the labeling agent can interact with different cells, cell populations or samples, allowing a particular report oligonucleotide to indicate a particular cell population (or cell or sample) and cell feature. In this way, different samples or groups can be independently processed and subsequently combined together for pooled analysis (e.g., partition-based barcoding as described elsewhere herein). Sec, e.g., U.S. Pat. Pub. 20190323088.

In some embodiments, to facilitate sample multiplexing, individual samples can be stained with lipid tags, such as cholesterol-modified oligonucleotides (CMOs, see, e.g., FIG. 7), anti-calcium channel antibodies, or anti-ACTB antibodies. Non-limiting examples of anti-calcium channel antibodies include anti-KCNN4 antibodies, anti-BK channel beta 3 antibodies, anti-a1B calcium channel antibodies, and anti-CACNA1A antibodies. Examples of anti-ACTB antibodies suitable for the methods of the disclosure include, but are not limited to, mAbGEa, ACTN05, AC-15, 15G5A11/E2, BA3R, and HHF35.

As described elsewhere herein, libraries of labelling agents can be associated with a particular cell feature as well as be used to identify analytes as originating from a particular cell population, or sample. Cell populations can be incubated with a plurality of libraries such that a cell or cells include multiple labelling agents. For example, a cell can include coupled thereto a lipophilic labeling agent and an antibody. The lipophilic labeling agent can indicate that the cell is a member of a particular cell sample, whereas the antibody can indicate that the cell includes a particular analyte. In this manner, the reporter oligonucleotides and labelling agents can allow multi-analyte, multiplexed analyses to be performed.

In some instances, these reporter oligonucleotides can include nucleic acid barcode sequences that permit identification of the labelling agent which the reporter oligonucleotide is coupled to. The use of oligonucleotides as the reporter can provide advantages of being able to generate significant diversity in terms of sequence, while also being readily attachable to most biomolecules, e.g., antibodies, etc., as well as being readily detected, e.g., using sequencing or array technologies.

Attachment (coupling) of the reporter oligonucleotides to the labelling agents can be achieved through any of a variety of direct or indirect, covalent or non-covalent associations or attachments. For example, reporter oligonucleotides can be covalently attached to a portion of a labelling agent (such a protein, e.g., an antigen or antigen fragment, an antibody or antibody fragment) using chemical conjugation techniques (e.g., Lightning-Link® antibody labelling kits available from Innova Biosciences), as well as other non-covalent attachment mechanisms, e.g., using biotinylated antibodies (or biotinylated antigens, or biotinylated antigen fragments) and oligonucleotides (or beads that include one or more biotinylated linker, coupled to oligonucleotides) with an avidin or streptavidin linker. Antibody and oligonucleotide biotinylation techniques are available. Scc, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31 (2): 708-715. Likewise, protein and peptide biotinylation techniques have been developed and are readily available. Sec, e.g., U.S. Pat. No. 6,265,552. Furthermore, click reaction chemistry such as a Methyltetrazine-PEG5-NHS Ester reaction, a TCO-PEG4-NHS Ester reaction, or the like, can be used to couple reporter oligonucleotides to labelling agents. Commercially available kits, such as those from Thunderlink and Abcam, and techniques common in the art can be used to couple reporter oligonucleotides to labelling agents as appropriate. In another example, a labelling agent is indirectly (e.g., via hybridization) coupled to a reporter oligonucleotide including a barcode sequence that identifies the label agent. For instance, the labelling agent can be directly coupled (e.g., covalently bound) to a hybridization oligonucleotide that includes a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides are releasable from the labelling agent, such as upon application of a stimulus. For example, the reporter oligonucleotide can be attached to the labeling agent through a labile bond (e.g., chemically labile, photolabile, thermally labile, etc.) as generally described for releasing molecules from supports elsewhere herein. In some instances, the reporter oligonucleotides described herein can include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, a sequencing primer or primer biding sequence (such as an R1, R2, or partial R1 or R2 sequence).

In some cases, the labelling agent (e.g., an antigen, an antigen fragment, an antibody, an antibody fragment) is presented as a monomer. In some cases, the labelling agent is presented as a multimer. In some cases, a labelling agent (e.g., an antigen, an antigen fragment, an antibody, an antibody fragment) is presented as a dimer. In some cases, a labelling agent (e.g., an antigen, an antigen fragment, an antibody, an antibody fragment) is presented as a trimer. In some cases, a labelling agent (e.g., an antigen, an antigen fragment, an antibody, an antibody fragment) is presented as a tetramer. In some cases, a labelling agent (e.g., an antigen, an antigen fragment, an antibody, an antibody fragment) is presented as a pentamer. In some cases, a labelling agent (e.g., an antigen, an antigen fragment, an antibody, an antibody fragment) is presented as a hexamer. In some cases, a labelling agent (e.g., an antigen, an antigen fragment, an antibody, an antibody fragment) is presented as a heptamer. In some cases, a labelling agent (e.g., an antigen, an antigen fragment, an antibody, an antibody fragment) is presented as an octamer. In some cases, a labelling agent (e.g., an antigen, an antigen fragment, an antibody, an antibody fragment) is presented as a nonamer. In some cases, a labelling agent (e.g., an antigen, an antigen fragment, an antibody, an antibody fragment) is presented as a decamer. In some cases, a labellign agent (.g., an antigen, an antigen fragment, an antibody, an antibody fragment) is presented as a 10+-mer.

In some cases, the labelling agent can include a reporter oligonucleotide and a label. A label can be fluorophore, a radioisotope, a molecule capable of a colorimetric reaction, a magnetic particle, or any other suitable molecule or compound capable of detection. The label can be conjugated to a labelling agent (or reporter oligonucleotide) either directly or indirectly (e.g., the label can be conjugated to a molecule that can bind to the labelling agent or reporter oligonucleotide). In some cases, a label is conjugated to an oligonucleotide that is complementary to a sequence of the reporter oligonucleotide, and the oligonucleotide can be allowed to hybridize to the reporter oligonucleotide.

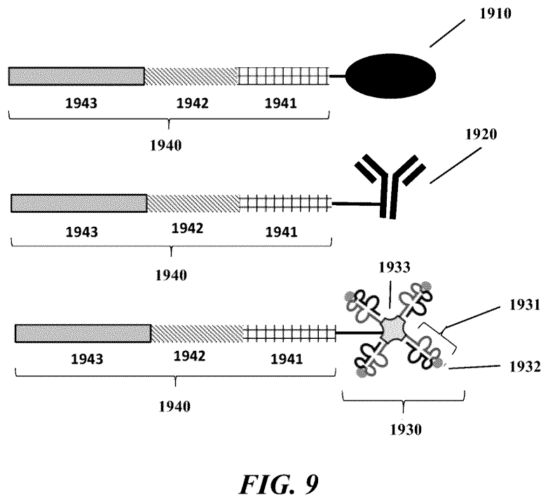

FIG. 9 describes exemplary labelling agents (1910, 1920, 1930) including reporter oligonucleotides (1940) attached thereto. Labelling agent 1910 (e.g., any of the labelling agents described herein) is attached (either directly, e.g., covalently attached, or indirectly) to reporter oligonucleotide 1940. Reporter oligonucleotide 1940 can include barcode sequence 1942 that identifies labelling agent 1910. Reporter oligonucleotide 1940 can also include one or more functional sequences 1943 that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, or a sequencing primer or primer binding sequence (such as an R1, R2, or partial R1 or R2 sequence).

Referring to FIG. 9, in some instances, reporter oligonucleotide 1940 conjugated to a labelling agent (e.g., 1910, 1920, 1930) includes a functional sequence 1941, a reporter barcode sequence 1942 that identifies the labelling agent (e.g., 1910, 1920, 1930), and reporter capture handle 1943. Reporter capture handle sequence 1943 can be configured to hybridize to a complementary sequence, such as a complementary sequence present on a nucleic acid barcode molecule 1990 (not shown), such as those described elsewhere herein. In some instances, nucleic acid barcode molecule 1990 is attached to a support (e.g., a bead, such as a gel bead), such as those described elsewhere herein. For example, nucleic acid barcode molecule 1990 can be attached to the support via a releasable linkage (e.g., including a labile bond), such as those described elsewhere herein. In some instances, reporter oligonucleotide 1940 includes one or more additional functional sequences, such as those described above.

In some instances, the labelling agent 1910 is a protein or polypeptide (e.g., an antigen or prospective antigen, or a fragment of an antigen or prospective antigen) including reporter oligonucleotide 1940. Reporter oligonucleotide 1940 includes reporter barcode sequence 1942 that identifies polypeptide 1910 and can be used to infer the presence of an analyte, e.g., a binding partner of polypeptide 1910 (i.e., a molecule or compound to which polypeptide 1910 can bind). In some instances, the labelling agent 1910 is a lipophilic moiety (e.g., cholesterol) including reporter oligonucleotide 1940, where the lipophilic moiety is selected such that labelling agent 710 integrates into a membrane of a cell or nucleus. Reporter oligonucleotide 740 includes reporter barcode sequence 742 that identifies lipophilic moiety 1910 which in some instances is used to tag cells (e.g., groups of cells, cell samples, etc.) and can be used for multiplex analyses as described elsewhere herein. In some instances, the labelling agent is an antibody 1920 (or an epitope binding fragment thereof) including reporter oligonucleotide 1940. Reporter oligonucleotide 1940 includes reporter barcode sequence 1942 that identifies antibody 1920 and can be used to infer the presence of, e.g., a target of antibody 1920 (i.e., a molecule or compound to which antibody 1920 binds). In other embodiments, labelling agent 1930 includes an MHC molecule 1931 including peptide 1932 and reporter oligonucleotide 1940 that identifies peptide 1932. In some instances, the MHC molecule is coupled to a support 1933. In some instances, support 1933 can be a polypeptide, such as streptavidin, or a polysaccharide, such as dextran. In some instances, reporter oligonucleotide 1940 can be directly or indirectly coupled to MHC labelling agent 1930 in any suitable manner. For example, reporter oligonucleotide 1940 can be coupled to MHC molecule 1931, support 1933, or peptide 1932. In some embodiments, labelling agent 1930 includes a plurality of MHC molecules, (e.g. is an MHC multimer, which can be coupled to a support (e.g., 1933)). There are many possible configurations of Class I and/or Class II MHC multimers that can be utilized with the compositions, methods, and systems disclosed herein, e.g., MHC tetramers, MHC pentamers (MHC assembled via a coiled-coil domain, e.g., Pro5® MHC Class I Pentamers, (ProImmune, Ltd.), MHC octamers, MHC dodecamers, MHC decorated dextran molecules (e.g., MHC Dextramer® (Immudex)), etc. For a description of exemplary labelling agents, including antibody and MHC-based labelling agents, reporter oligonucleotides, and methods of use, see, e.g., U.S. Pat. No. 10,550,429 and U.S. Pat. Pub. 20190367969.

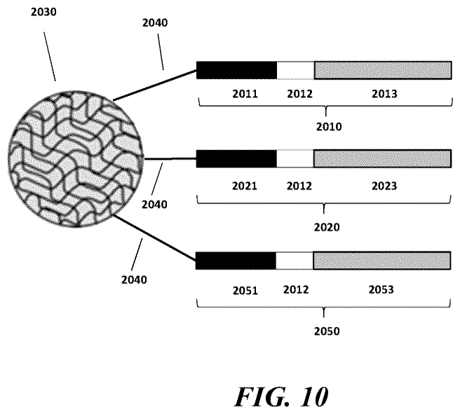

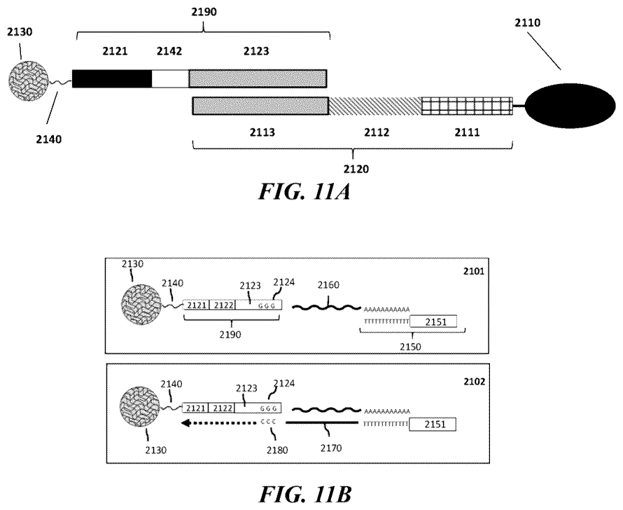

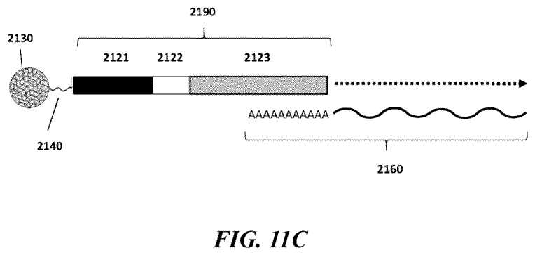

Exemplary barcode molecules attached to a support (e.g., a bead) is shown in FIG. 10. In some embodiments, analysis of multiple analytes (e.g., RNA and one or more analytes using labelling agents described herein) can include nucleic acid barcode molecules as generally depicted in FIG. 10. In some embodiments, nucleic acid barcode molecules 2010 and 2020 are attached to support 2030 via a releasable linkage 2040 (e.g., including a labile bond) as described elsewhere herein. Nucleic acid barcode molecule 2010 can include functional sequence 2011, barcode sequence 2012 and capture sequence 2013. Nucleic acid barcode molecule 2020 can include adapter sequence 2021, barcode sequence 2012, and capture sequence 2023, wherein capture sequence 2023 includes a different sequence than capture sequence 2013. In some instances, adapter 2011 and adapter 2021 include the same sequence. In some instances, adapter 2011 and adapter 2021 include different sequences. Although support 2030 is shown including nucleic acid barcode molecules 2010 and 2020, any suitable number of barcode molecules including common barcode sequence 2012 are contemplated herein. For example, in some embodiments, support 2030 further includes nucleic acid barcode molecule 2050. Nucleic acid barcode molecule 2050 can include adapter sequence 2051, barcode sequence 2012 and capture sequence 2053, wherein capture sequence 2053 includes a different sequence than capture sequence 2013 and 2023. In some instances, nucleic acid barcode molecules (e.g., 2010, 2020, 2050) include one or more additional functional sequences, such as a UMI or other sequences described herein. The nucleic acid barcode molecules 2010, 2020 or 2050 can interact with analytes as described elsewhere herein, for example, as depicted in FIGS. 11A-11C.

Referring to FIG. 11A, in an instance where cells are labelled with labeling agents, capture sequence 2123 can be complementary to an adapter sequence of a reporter oligonucleotide. Cells can be contacted with one or more reporter oligonucleotide 2120 conjugated labelling agents 2110 (e.g., polypeptide such as an antigen or fragment of an antigen, antibody, or others described elsewhere herein). In some cases, the cells can be further processed prior to barcoding. For example, such processing steps can include one or more washing and/or cell sorting steps. In some instances, a cell that is bound to labelling agent 2110 which is conjugated to oligonucleotide 2120 and support 2130 (e.g., a bead, such as a gel bead) including nucleic acid barcode molecule 2190 is partitioned into a partition amongst a plurality of partitions (e.g., a droplet of a droplet emulsion or a well of a microwell array). In some instances, the partition includes at most a single cell bound to labelling agent 2110. In some instances, reporter oligonucleotide 2120 conjugated to labelling agent 2110 (e.g., polypeptide such as an antigen or fragment of an antigen, an antibody, pMHC molecule such as an MHC multimer, etc.) includes a first functional sequence 2111 (e.g., a primer sequence), a barcode sequence 2112 that identifies the labelling agent 2110 (e.g., the polypeptide such as an antigen or fragment of an antigen, antibody, or peptide of a pMHC molecule or complex), and a capture handle sequence 2113. Capture handle sequence 2113 can be configured to hybridize to a complementary sequence, such as capture sequence 2123 present on a nucleic acid barcode molecule 2190 (e.g., partition-specific barcode molecule). In some instances, oligonucleotide 2110 includes one or more additional functional sequences, such as those described elsewhere herein.

Barcoded nucleic acid molecules can be generated (e.g., via a nucleic acid reaction, such as nucleic acid extension, reverse transcription, or ligation) from the constructs described in FIGS. 11A-11C. For example, capture handle sequence 2113 can then be hybridized to complementary capture sequence 2123 to generate (e.g., via a nucleic acid reaction, such as nucleic acid extension or ligation) a barcoded nucleic acid molecule including cell barcode (e.g., common barcode or partition-specific barcode) sequence 2122 (or a reverse complement thereof) and reporter barcode sequence 2112 (or a reverse complement thereof). In some embodiments, the nucleic acid barcode molecule 2190 (e.g., partition-specific barcode molecule) further includes a UMI. Barcoded nucleic acid molecules can then be optionally processed as described elsewhere herein, e.g., to amplify the molecules and/or append sequencing platform specific sequences to the fragments. Scc, e.g., U.S. Pat. Pub. 2018/0105808. Barcoded nucleic acid molecules, or derivatives generated therefrom, can then be sequenced on a suitable sequencing platform.

In some instances, analysis of multiple analytes (e.g., nucleic acids and one or more analytes using labelling agents described herein) can be performed. For example, the workflow can include a workflow as generally depicted in any of FIGS. 11A-11C, or a combination of workflows for an individual analyte, as described elsewhere herein. For example, by using a combination of the workflows as generally depicted in FIGS. 11A-11C, multiple analytes can be analyzed.

In some instances, analysis of an analyte (e.g. a nucleic acid, a polypeptide, a carbohydrate, a lipid, etc.) includes a workflow as generally depicted in FIG. 11A. A nucleic acid barcode molecule 2190 can be co-partitioned with the one or more analytes. In some instances, nucleic acid barcode molecule 2190 is attached to a support 2130 (e.g., a bead, such as a gel bead), such as those described elsewhere herein. For example, nucleic acid barcode molecule 2190 can be attached to support 2130 via a releasable linkage 2140 (e.g., including a labile bond), such as those described elsewhere herein. Nucleic acid barcode molecule 2190 can include a functional sequence 2121 and optionally include other additional sequences, for example, a barcode sequence 2122 (e.g., common barcode, partition-specific barcode, or other functional sequences described elsewhere herein), and/or a UMI sequence 2125. The nucleic acid barcode molecule 2190 can include a capture sequence 2123 that can be complementary to another nucleic acid sequence, such that it can hybridize to a particular sequence.

For example, capture sequence 2123 can include a poly-T sequence and can be used to hybridize to mRNA. Referring to FIG. 11C, in some embodiments, nucleic acid barcode molecule 2190 includes capture sequence 2123 complementary to a sequence of RNA molecule 2160 from a cell. In some instances, capture sequence 2123 includes a sequence specific for an RNA molecule. Capture sequence 2123 can include a known or targeted sequence or a random sequence. In some instances, a nucleic acid extension reaction can be performed, thereby generating a barcoded nucleic acid product including capture sequence 2123, the functional sequence 2121, UMI sequence 2125, any other functional sequence, and a sequence corresponding to the RNA molecule 2160.

In another example, capture sequence 2123 can be complementary to an overhang sequence or an adapter sequence that has been appended to an analyte. For example, referring to FIG. 11B, in some embodiments, primer 2150 includes a sequence complementary to a sequence of nucleic acid molecule 2160 (such as an RNA encoding for a BCR sequence) from a biological particle. In some instances, primer 2150 includes one or more sequences 2151 that are not complementary to RNA molecule 2160. Sequence 2151 can be a functional sequence as described elsewhere herein, for example, an adapter sequence, a sequencing primer sequence, or a sequence the facilitates coupling to a flow cell of a sequencer. In some instances, primer 2150 includes a poly-T sequence. In some instances, primer 2150 includes a sequence complementary to a target sequence in an RNA molecule. In some instances, primer 2150 includes a sequence complementary to a region of an immune molecule, such as the constant region of a BCR sequence. Primer 2150 is hybridized to nucleic acid molecule 2160 and complementary molecule 2170 is generated. For example, complementary molecule 2170 can be cDNA generated in a reverse transcription reaction. In some instances, an additional sequence can be appended to complementary molecule 2170. For example, the reverse transcriptase enzyme can be selected such that several non-templated bases 2180 (e.g., a poly-C sequence) are appended to the cDNA. In another example, a terminal transferase can also be used to append the additional sequence. Nucleic acid barcode molecule 2190 includes a sequence 2124 complementary to the non-templated bases, and the reverse transcriptase performs a template switching reaction onto nucleic acid barcode molecule 2190 to generate a barcoded nucleic acid molecule including cell (e.g., partition specific) barcode sequence 2122 (or a reverse complement thereof) and a sequence of complementary molecule 2170 (or a portion thereof). In some instances, capture sequence 2123 includes a sequence complementary to a region of an immune molecule, such as the constant region of a BCR sequence. Capture sequence 2123 is hybridized to nucleic acid molecule 2160 and a complementary molecule 2170 is generated. For example, complementary molecule 2170 can be generated in a reverse transcription reaction generating a barcoded nucleic acid molecule including cell barcode (e.g., common barcode or partition-specific barcode) sequence 2122 (or a reverse complement thereof) and a sequence of complementary molecule 2170 (or a portion thereof). Additional methods and compositions suitable for barcoding cDNA generated from mRNA transcripts including those encoding V (D) J regions of an immune cell receptor and/or barcoding methods and composition including a template switch oligonucleotide are described in International Patent Application WO2018/075693, U.S. Patent Publication No. 2018/0105808, U.S. Patent Publication No. 2015/0376609, filed Jun. 26, 2015, and U.S. Patent Publication No. 2019/0367969.

### Combinatorial Barcoding

In some instances, barcoding of a nucleic acid molecule may be done using a combinatorial approach. In such instances, one or more nucleic acid molecules (which may be comprised in a biological particle, e.g., a cell, e.g., a fixed cell, organelle, nucleus, or cell bead) may be partitioned (e.g., in a first set of partitions, e.g., wells or droplets) with one or more first nucleic acid barcode molecules (optionally coupled to a bead). The first nucleic acid barcode molecules or derivative thereof (e.g., complement, reverse complement) may then be attached to the one or more nucleic acid molecules, thereby generating barcoded nucleic acid molecules, e.g., using the processes described herein. The first nucleic acid barcode molecules may be partitioned to the first set of partitions such that a nucleic acid barcode molecule, of the first nucleic acid barcode molecules, that is in a partition comprises a barcode sequence that is unique to the partition among the first set of partitions. Each partition may comprise a unique barcode sequence. For example, a set of first nucleic acid barcode molecules partitioned to a first partition in the first set of partitions may each comprise a common barcode sequence that is unique to the first partition among the first set of partitions, and a second set of first nucleic acid barcode molecules partitioned to a second partition in the first set of partitions may each comprise another common barcode sequence that is unique to the second partition among the first set of partitions. Such barcode sequence (unique to the partition) may be useful in determining the cell or partition from which the one or more nucleic acid molecules (or derivatives thereof) originated.

The barcoded nucleic acid molecules from multiple partitions of the first set of partitions may be pooled and re-partitioned (e.g., in a second set of partitions, e.g., one or more wells or droplets) with one or more second nucleic acid barcode molecules. The second nucleic acid barcode molecules or derivative thereof may then be attached to the barcoded nucleic acid molecules. As with the first nucleic acid barcode molecules during the first round of partitioning, the second nucleic acid barcode molecules may be partitioned to the second set of partitions such that a nucleic acid barcode molecule, of the second nucleic acid barcode molecules, that is in a partition comprises a barcode sequence that is unique to the partition among the second set of partitions. Such barcode sequence may also be useful in determining the cell or partition from which the one or more nucleic acid molecules or first barcoded nucleic acid molecules originated. The barcoded nucleic acid molecules may thus comprise two barcode sequences (e.g., from the first nucleic acid barcode molecules and the second nucleic acid barcode molecules).

Additional barcode sequences may be attached to the barcoded nucleic acid molecules by repeating the processes any number of times (e.g., in a split-and-pool approach), thereby combinatorically synthesizing unique barcode sequences to barcode the one or more nucleic acid molecules. For example, combinatorial barcoding may comprise at least 1, 2, 3, 4, 5, 6, 7, 8, 9, 10 or more operations of splitting (e.g., partitioning) and/or pooling (e.g., from the partitions). Additional examples of combinatorial barcoding may also be found in International Patent Publication Nos. WO2019/165318, each of which is herein entirely incorporated by reference for all purposes.

Beneficially, the combinatorial barcode approach may be useful for generating greater barcode diversity, and synthesizing unique barcode sequences on nucleic acid molecules derived from a cell or partition. For example, combinatorial barcoding comprising three operations, each with 100 partitions, may yield up to 106 unique barcode combinations. In some instances, the combinatorial barcode approach may be helpful in determining whether a partition contained only one cell or more than one cell. For instance, the sequences of the first nucleic acid barcode molecule and the second nucleic acid barcode molecule may be used to determine whether a partition comprised more than one cell. For instance, if two nucleic acid molecules comprise different first barcode sequences but the same second barcode sequences, it may be inferred that the second set of partitions comprised two or more cells.

In some instances, combinatorial barcoding may be achieved in the same compartment. For instance, a unique nucleic acid molecule comprising one or more nucleic acid bases may be attached to a nucleic acid molecule (e.g . . . a sample or target nucleic acid molecule) in successive operations within a partition (e.g . . . droplet or well) to generate a barcoded nucleic acid molecule. A second unique nucleic acid molecule comprising one or more nucleic acid bases may be attached to the barcoded nucleic acid molecule molecule. In some instances, all the reagents for barcoding and generating combinatorially barcoded molecules may be provided in a single reaction mixture, or the reagents may be provided sequentially.

In some instances, cell beads comprising nucleic acid molecules may be barcoded. Methods and systems for barcoding cell beads are further described in PCT/US2018/067356 and U.S. Pat. Pub. No. 2019/0330694, which are hereby incorporated by reference in its entirety.

### Scipio/CS Genetics

In some instances wherein a partition is a volume wherein diffusion of contents beyond the volume is inhibited, the partition contains a diffusion resistant material. Such partition may also be referred to herein as a diffusion resistant partition. The diffusion resistant material may have an increased viscosity. The diffusion resistant material may be or comprise a matrix, e.g., a polymeric matrix, or a gel. Suitable polymers or gels are disclosed herein. The matrix can be a porous matrix capable of entraining and/or retaining materials within its matrix. In some embodiments, a diffusion resistant partition comprises a single biological particle and a single bead, the single bead comprising a plurality of nucleic acid barcode molecules comprising a partition specific barcode sequence. In some embodiments the partition specific barcode sequence is unique to the diffusion resistant partition. In some embodiments, partitioning comprises contacting a plurality of biological particles with a plurality of beads in a diffusion resistant material to provide a diffusion resistant partition comprising a single biological particle and a single bead. In some embodiments, partitioning comprises contacting a plurality of biological particles with a plurality of beads in a liquid comprising a polymeric precursor material that may be capable of being formed into a gel or other solid or semi-solid matrix, and subjecting the liquid to conditions sufficient to polymerize or gel the precursors, e.g., as described herein. In some embodiments, the biological particle may be lysed or permeabilized in the diffusion resistant partition. In some embodiments, a nucleic acid analyte of the biological particle (which may include a reporter oligonucleotide associated with a labelling agent disclosed herein) may be coupled with a nucleic acid barcode molecule in the diffusion resistant partition. In some cases, further processing, e.g., generation of barcoded nucleic acid molecules, may be performed in the diffusion resistant partition or in bulk. For example, nucleic acid analytes, once coupled to nucleic acid barcode molecules in partitions, may be pooled and then subjected to further processing in bulk (e.g, extension, reverse transcription, or other processing) to generate barcoded nucleic acid molecules. For other example, nucleic acid analytes, one coupled to nucleic acid barcode molecules in diffusion resistant partitions, may be subjected to further processing in the diffusion resistant partitions to generate barcoded nucleic acid molecules.

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

No admission is made that any reference cited herein constitutes prior art. The discussion of the references states what their authors assert, and the Applicant reserves the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

Additional embodiments are disclosed in further detail in the following examples, which are provided by way of illustration and are not in any way intended to limit the scope of this disclosure or the claims.

### EXAMPLES

### Example 1: Analysis of CAR T Cells with Targeted Gene Expression

Single cell barcoded gene expression libraries were prepared as follows: Briefly, transduced anti-CD19 CAR T cells were acquired from one donor from a commercial vendor and these cells were thawed following recommended protocols on the 10× Genomics website (8). As explained further below, the CAR T cell sample was 45% viable after thawing and washing. The scFv region of the CAR vector enables successfully transduced CAR T cells to bind CD19 on target cells. In some experiments (Example 3), CAR T cells were titrated into PBMCs from an unrelated donor at 0.1%, 1%, 5%, and 10% of the total cell number. In some experiments (Example 2), a sample of 100% CAR T cells was also run to further dissect the cellular composition of the CAR T cell product.

Libraries were then generated targeting 5,000 recovered cells for each sample condition using the Chromium Single Cell Immune Profiling (v2) and Targeted Gene Expression solutions (9, 10). The targeted gene expression profiling used the Human Immunology Panel (described in https://www.10xgenomics.com/support/single-cell-gene-expression/documentation/steps/targeted-gene-expression/human-immunology-panel) and an add-on custom bait set targeting the single-chain variable fragment (scFv) region of the CAR vector.

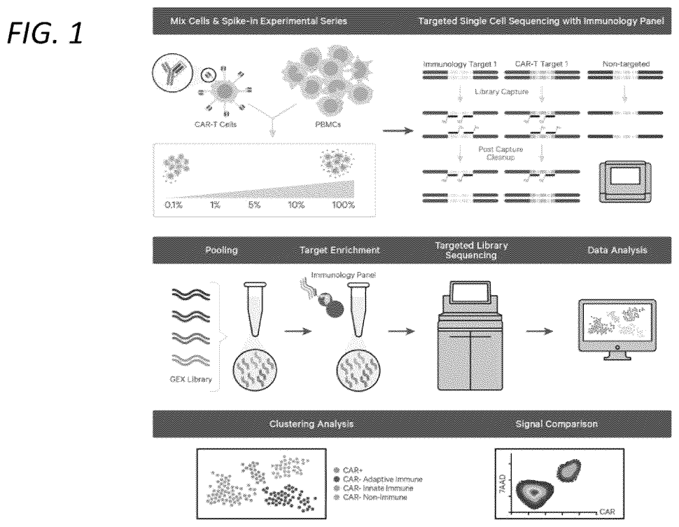

As shown in FIG. 1, the following three libraries were generated: a targeted gene expression library enriched for immune pathway genes (generated as described above using the Human Immunology Panel according to manufacturer's instructions), a targeted gene expression library enriched for CAR sequences (generated using the add-on custom bait set targeting the single-chain variable fragment (scFv) encoding portion of the CAR), and a non-targeted library (unenriched).

A general workflow for preparation of the single cell barcoded gene expression libraries, sequencing, and analysis is shown in FIG. 1.

The custom bait set targeting the scFv region of the CAR vector included at least one biotinylated bait complementary to a scFv encoding region of the CD19 CAR construct.

In some experiments, the custom bait set targeting the scFv region of the CAR vector included multiple biotinylated baits complementary to different regions of the scFv encoding portion of the CD19 CAR construct.

In some experiments, the concentration of baits in each of the targeted panels was designed to maximize sequencing saturation of the targeted genes in the panel. In order to achieve this, for each panel, the concentrations of the baits were selected such that targeted genes with high expression levels would be recovered slightly less efficiently (without affecting biological significance) which in turn allows targeted genes with low expression to be recovered significantly more efficiently.

Sequences of baits targeting the scFv encoding portion of the CD19 CAR construct are shown in Table 1.

TABLE 1

| | SEQ ID |
|---|---|
| Sequence | NO. |
| GACATCCAGATGACACAGACTACATCCTCC | 1 |
| CTGTCTGCCTCTCTGGGAGACAGAGTCACC | |
| ATCAGTTGCAGGGCAAGTCAGGACATTAGT | |
| AAATATTTAAATTGGTATCAGCAGAAACCA | |
| GATGGAACTGTTAAACTCCTGATCTACCAT | 2 |
| ACATCAAGATTACACTCAGGAGTCCCATCA | |
| AGGTTCAGTGGCAGTGGGTCTGGAACAGAT | |
| TATTCTCTCACCATTAGCAACCTGGAGCAA | |
| GAAGATATTGCCACTTACTTTTGCCAACAG | 3 |
| GGTAATACGCTTCCGTACACGTTCGGAGGG | |
| GGGACTAAGTTGGAAATAACAGGTGGCGGT | |
| GGCAGCGGCGGTGGTGGTTCCGGAGGCGGC | |
| GAGGTGAAACTGCAGGAGTCAGGACCTGGC | 4 |
| CTGGTGGCGCCCTCACAGAGCCTGTCCGTC | |
| ACATGCACTGTCTCAGGGGTCTCATTACCC | |
| GACTATGGTGTAAGCTGGATTCGCCAGCCT | |
| CCACGAAAGGGTCTGGAGTGGCTGGGAGTA | 5 |
| ATATGGGGTAGTGAAACCACATACTATAAT | |
| TCAGCTCTCAAATCCAGACTGACCATCATC | |
| AAGGACAACTCCAAGAGCCAAGTTTTCTTA | |
| AAAATGAACAGTCTGCAAACTGATGACACA | 6 |
| GCCATTTACTACTGTGCCAAACATTATTAC | |
| TACGGTGGTAGCTATGCTATGGACTACTGG | |
| GGTCAAGGAACCTCAGTCACCGTCTCCTCA | |

In some experiments, the custom bait set included additional baits to gene targets associated with CAR expressing cells, such as those selected from the group consisting of ABCA1, ACP5, ADAMDECI, ADM, AK8, ANKRD22, ARRDC4, BCL7A, C15orf48, CD226, CDA, CEACAM3, CIITA, CLEC12A, CRABP2, CSF2RA, CST3, CSTA, CSTB, CTSB, CYP1B1, ENPP3, EPB41L3, EREG, FBP1, FGR, FTL, GGT5, GPNMB, HAVCR1, HLX, ID3, IGSF6, IL411, INHBA, KYNU, LY2, MAFB, MMP14, MNDA, Ms4A7, NCF1, OSCAR, PDLIM4, PILRA, PLA2G7, PLBD1, PTAFR, NECTIN2, RAB13, RBM47, RC3H2, RC3H1, SDC2, SLAMF8, SLC15A3, SLC1A3, SLC43A2, SOD2, SPI1, TCF3, TFEC, TGM2, TMEM176A, TMEM176B, TNFAIP2, TNFAIP6, TNS3, TXN, TYROBP, and ZNF385A.

In some experiments, the hybridization of baits was carried out at 65° C. for 2 hours. Unbound baits were removed using washes at 65° C.

The libraries (e.g., the targeted gene expression libraries and the unenriched library) were sequenced (e.g., to a depth of 10,000 read pairs per cell on a NovaSeq 6000), as shown in FIG. 1.

### Example 2: Improved Resolution of Phenotypic Diversity with Targeted Gene Expression

In this experiment, it is shown that vendor-provided flow cytometry data can distinguish between non-transduced and transduced cells, but with less resolution than single cell analysis due to the lack of clean separation between CAR-positive (CAR+) and CAR-negative (CAR−) cell populations.

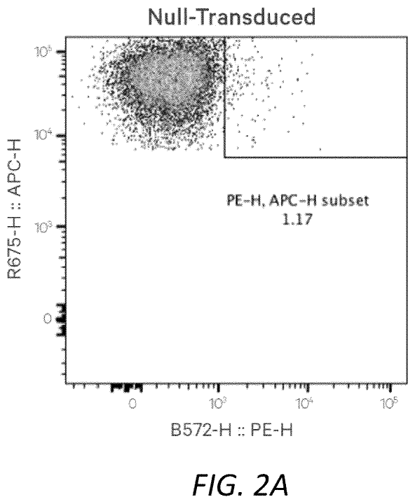

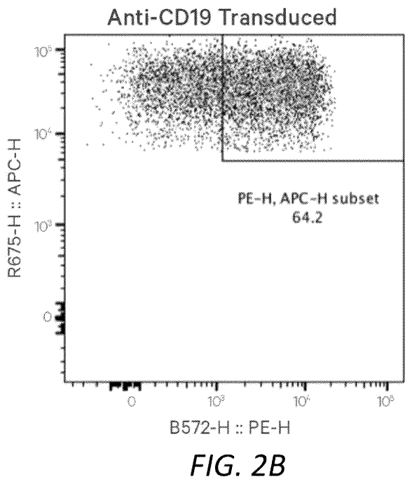

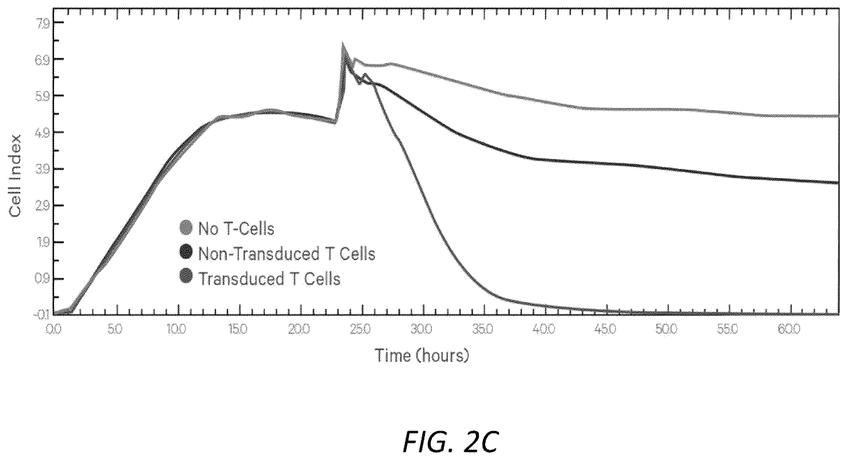

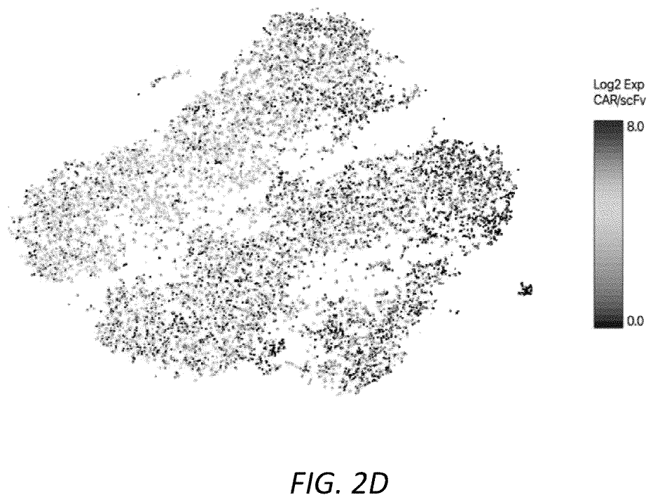

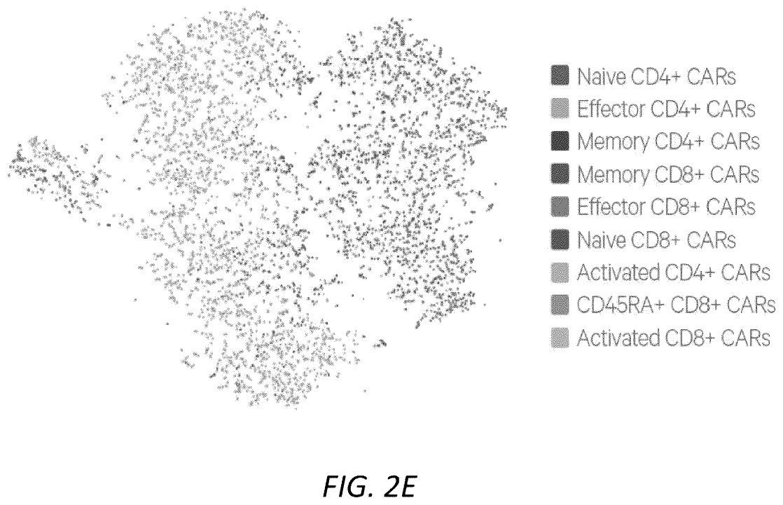

FIG. 2A depicts flow analysis of null-transduced cells by the vendor. Donor PBMCs were transduced with a CAR-null vector lacking the anti-CD19 CAR and the accompanying FLAG-tag. Data presented here show that no detectable anti-CD19 CAR signal is present in null-transduced cells from the donor. X axis: anti-FLAG-tag fluorescence. Y axis: anti-CD3 fluorescence.

FIG. 2B depicts gating of transduced cells by flow cytometry, as performed by the vendor. Donor PBMCs were transduced with the CAR-positive vector containing the anti-CD19 CAR and the accompanying FLAG-tag. Data presented here show efficient transduction of donor T cells with the anti-CD19 CAR vector. X axis: anti-FLAG-tag fluorescence. Y axis: anti-CD3 fluorescence.

FIG. 2C depicts a graphical representation of results from a killing assay. Incubation of CD19 presenting HeLa cells with a) no additional cells, b) non-transduced T cells, and c) CAR+ T cells (i.e., cells transduced with the CAR-positive vector) was performed. This data shows that the CAR+ T cells kill the transduced Hela cells quickly due to their targeting capacity. Collectively, FIGS. 2A-2C demonstrate that T cells that incorporated the CAR were also better able to kill CD19+ cells compared to non-transduced T cells.

FIG. 2D shows detection of anti-CD19 CAR expressed transgenes based on targeting baits (i.e., using barcoded libraries generated as described in Example 1) in a sample of 100% CAR T cells. When compared to flow, single cell analysis with Targeted Gene Expression was able to detect expression of the anti-CD19 scFv transgene based on its expressed transcript to a high degree among a population of 100% CAR T cell product. Approximately 42% of cells in the population exhibited expression of the CAR construct (FIG. 2D). It should be noted that Deng et al [Deng Q, et al. Characteristics of anti-CD19 CAR T cell infusion products associated with efficacy and toxicity in patients with large B cell lymphomas. Nat Med 26 (12): 1878-1887, 2020. doi: 10.1038/s41591-020-1061-7] also describes characterization of a starting sample of CAR T cells using 10×5′ immune profiling and an approach for preparing enriched libraries using biotinylated baits. However, the methods described herein (see, e.g., FIG. 1, Example 1) achieved superior sensitivity of detection (42%) as compared to the methods of Deng (where detection was only at 24%), despite several technical differences that would be expected to yield greater detection in Deng. For example, in experiments described herein, the custom bait set included at least one bait targeting the scFv encoding portion of the CAR construct. For other example, in Deng, the cells were at least 85% viable, whereas in experiments described herein, the cells were 45% viable. For yet other example, in Deng, the probe hybridization was carried out at much longer period of time (65° C. overnight) than the experimental methods of Example 1 (65° C. for 2 hours). For yet other example, the washes were less stringent in Deng (including both 65° C. and room temperature washes), compared to the experimental methods of Example 1 (all washes at 65° C.). Given the superior viability of the cell sample, the longer probe hybridization time, and the lesser stringency of washes (in Deng), the superior sensitivity of detection using the methods described herein is unexpected and surprising.

FIG. 2E shows CAR+ cell subclustering with cell type classification based on gene expression. When CAR+ T cell sub-clustering analysis with cell type classification based on gene expression of the targeted panel and bait set was performed, a wide range of different T cell types in which the CAR was incorporated was found, including naive, effector, memory, and activated CD4+ and CD8+ cells. The ability of single cell analysis to distinguish between different T cell subtypes and capture an unbiased measurement of CAR expression in the cellular product shows the power of this approach over flow cytometry (FIG. 2E).

Compared to flow cytometry, targeted gene expression (e.g., methods described herein, e.g., in Example 1 and FIG. 1) not only shows highly concordant cell type identification to flow, but further refines and resolves these cell types and their functional states.

### Example 3: Titration Analysis Demonstrates Superior Sensitivity of a Single Cell Approach

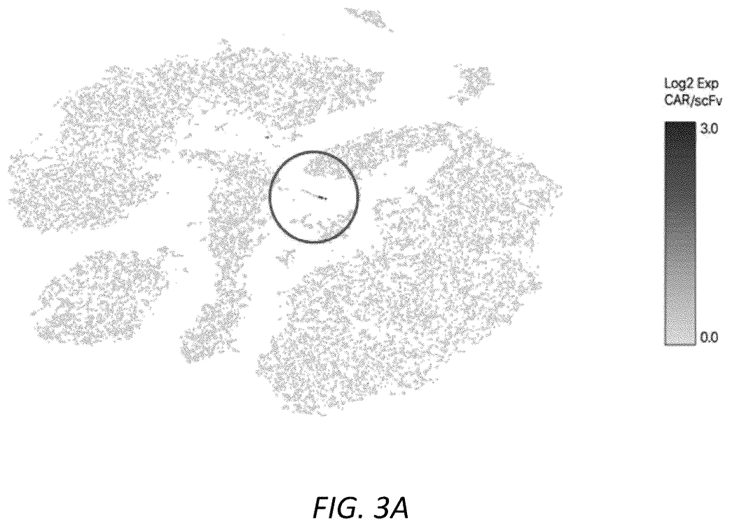

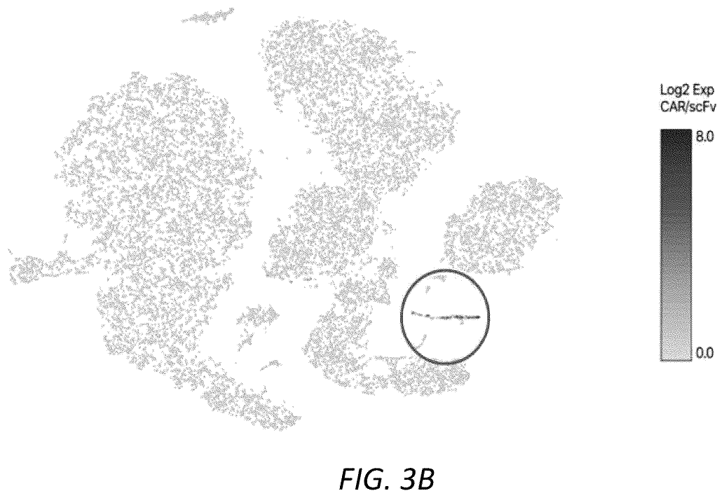

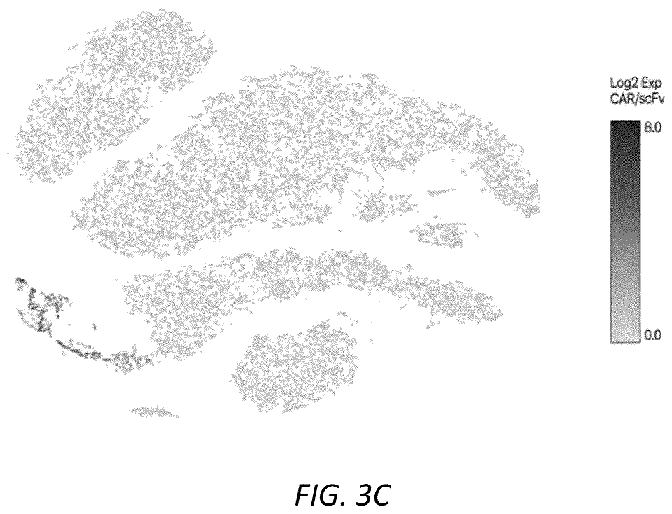

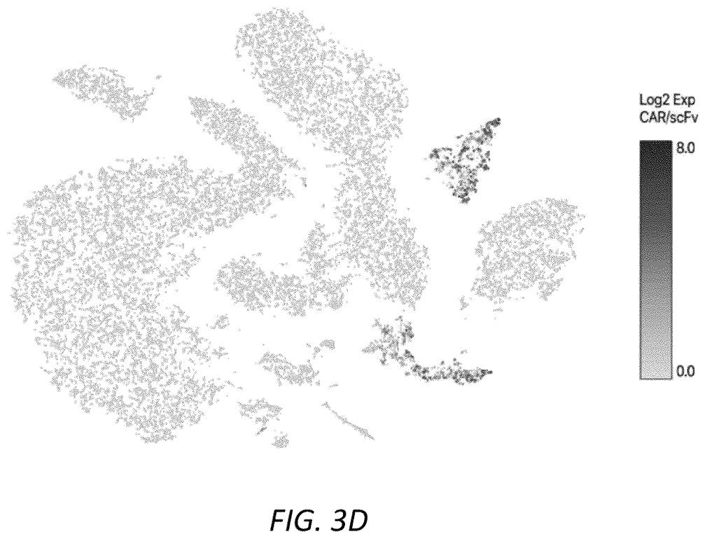

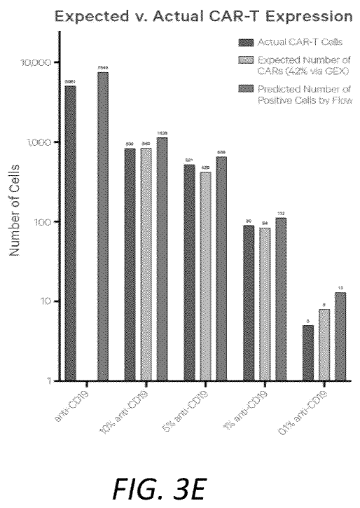

In order to test the limit of detection, CAR T cells were spiked into PBMCs at concentrations of 0.1%, 1%, 5%, and 10%, as described in Example 1. These data were analysed using Loupe Cell Browser 5.1.0 to gate CAR+ cells based on the presence and abundance of anti-CAR scFv unique molecular identifiers (UMIs). Expected and actual recoveries of CAR+ cells in this titration series showed that CAR+ cells could be detected with high sensitivity (FIGS. 3A-3E).

Using probes specific to the scFv region of the CAR and the targeted gene expression workflow provides the sensitivity needed to detect CAR+ and immune cells within a heterogeneous mixture of cells, which was achieved at a level of less than 0.01% (5 of 20,119 cells) for the lowest titration. This sensitivity is typically considered the lower limit of detection in flow cytometry, although the analysis would require at least 5 million cells due to the sample variability seen with lower percentages of cells. Given the vendor's placement of the gate used to select CAR+ cells upstream of our single cell transcriptomic experiments (FIGS. 2A-2B) this variability is not surprising.

It is important to note that the CAR T cell sample was 45% viable after thawing and washing (i.e. as described in Example 1). The high number of healthy PBMCs in the titration series offset the poor viability of the CAR T cell sample and did not affect the overall recovery. However, reduced recovery was observed, as expected, in the 100% CAR T cell sample where there were no PBMCs.

### Example 4: Targeted, Single Cell Transcriptomics Enables High Resolution Analysis

Characterizing the extent of CAR construct transduction at high resolution and with high sensitivity is essential to creating a durable infusion product and increasing the efficacy of CAR T-cell therapy. In this paper, it has been shown that using a targeted single cell approach enables product composition analyses of CAR-transduced cellular populations at higher resolution and sensitivity than currently employed technologies, such as flow cytometry.

Using Single Cell Immune Profiling (v2) and Targeted Gene Expression (10× Genomics), targeted, single cell transcriptomic analysis showed that we could detect CAR expression in 42% of the population while subclustering analysis detected CAR transduction in multiple different cell types and functional states. In short, a targeted single cell approach encompasses both the high sensitivity and resolution required for detecting a small number of cells expressing specific transcripts within a larger sample as well as resolving their type and functional states. This makes it a powerful tool for characterizing and optimizing CAR T-cell therapy infusion products.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1-56. (canceled)

57. A method of characterizing one or more immune cells, the method comprising:
a) partitioning a reaction mixture into a plurality of partitions, wherein the reaction mixture comprises a population of immune cells, wherein an immune cell of the population of immune cells comprises a plurality of nucleic acid molecules comprising endogenous sequences and an exogenous chimeric antigen receptor (CAR) sequence, wherein the exogenous CAR sequence comprises a single-chain fragment variable (scFv) encoding portion,
wherein a partition of the plurality of partitions comprises:
(i) the immune cell, and
(ii) a plurality of nucleic acid barcode molecules comprising a partition-specific barcode sequence;
b) generating a plurality of barcoded nucleic acid molecules, wherein the barcoded nucleic acid molecules comprise:
(i) a first barcoded nucleic acid molecule comprising a sequence corresponding to the exogenous CAR sequence or reverse complement thereof, and the partition-specific barcode sequence; and
(ii) a second barcoded nucleic acid molecule comprising a sequence corresponding to an endogenous sequence or reverse complement thereof, and the partition-specific barcode sequence;
c) using a nucleic acid bait capable of hybridizing to the scFv encoding portion of the exogenous CAR sequence to enrich a subset of the barcoded nucleic acid molecules.

58. The method of claim 57, further comprising determining the sequences of the barcoded nucleic acid molecules (or derivatives thereof) from the enriched subset.

59. The method of claim 57, wherein the bait is biotinylated.

60. The method of claim 57, wherein (c) comprises hybridizing the bait to the scFv encoding portion of the first barcoded nucleic acid molecule or derivative thereof.

61. The method of claim 57, wherein (c) comprises associating the bait with a set of beads comprising a biotin-binding partner, thereby operatively coupling the first barcoded nucleic acid molecules or derivatives thereof with the set of beads.

62. The method of claim 61, wherein the biotin-binding partner is selected from the group consisting of streptavidin, avidin, deglycosylated avidin, traptavidin, tamavidin, xenavidin, bradavidin, AVR2 (avidin related protein 2), AVR4 (avidin related protein 4), and variants, mutants, derivatives, and homologs of any thereof.

63. The method of claim 61, wherein (c) comprises removing the barcoded nucleic acid molecules or derivatives thereof that are not operatively coupled to the set of beads, thereby providing the enriched subset of barcoded nucleic acid molecules.

64. The method of claim 57, further comprising providing multiple baits capable of hybridizing to different regions of the scFv encoding portion of the exogenous CAR sequence.

65. The method of claim 57, wherein the population of immune cells is an ex vivo population of immune cells.

66. The method of claim 57, wherein the immune cells are lymphocytes.

67. The method of claim 66, wherein the lymphocytes are
i) CD4+ T cells selected from the group consisting of naïve CD4+ T cells, effector CD4+ T cells, memory CD4+ T cells, and activated CD4+ T cells; and/or
ii) CD8+ T cells selected from the group consisting of naïve CD8+ T cells, effector CD8+ T cells, memory CD8+ T cells, CD45RA+ CD8+ T cells, and activated CD8+ T cells.

68. The method of claim 57, wherein the CAR-expressing immune cell comprises a CD19 single chain variable fragment (scFv), CD28, and CD3-gamma.

69. The method of claim 57, wherein the plurality of nucleic acid barcode molecules further comprises: i) a capture sequence configured to couple to an mRNA or DNA analyte, and/or ii) a unique molecular identifier (UMI) sequence, and/or iii) a functional sequence.

70. The method of claim 57, wherein the partition is a droplet, a micro-vesicle, a flow cell, a reaction chamber, a reaction compartment, a tube, a well, or a microwell.

71. The method of claim 57, wherein the partition is a droplet.

72. The method of claim 57, wherein the plurality of nucleic acid barcode molecules is provided as part of a support.

73. The method of claim 72, wherein the support is a bead.

74. The method of claim 73, wherein the bead is a gel bead.

75. A method of resolving heterogeneity in an infusion product, the method comprising:
a) partitioning a reaction mixture into a plurality of partitions, wherein the reaction mixture comprises:
(i) an infusion product comprising a population of immune cells comprising a chimeric antigen receptor (CAR)-expressing immune cell, wherein the CAR-expressing immune cell comprises a plurality of nucleic acid molecules comprising endogenous sequences and an exogenous CAR sequence;
wherein a partition of the plurality of partitions comprises:
(i) the CAR-expressing immune cell, and
(ii) a plurality of nucleic acid barcode molecules comprising a partition-specific barcode sequence;
b) in the partition, generating barcoded nucleic acid molecules, wherein the barcoded nucleic acid molecules comprise:
(i) a first barcoded nucleic acid molecule comprising a sequence corresponding to the exogenous CAR sequence or reverse complement thereof, and the partition-specific barcode sequence; and
(ii) a second barcoded nucleic acid molecule comprising a sequence corresponding to an endogenous sequence or reverse complement thereof, and the partition-specific barcode sequence; and
c) using a nucleic acid bait capable of hybridizing to the scFv encoding portion of the exogenous CAR sequence to enrich a subset of the barcoded nucleic acid molecules.

76. The method of claim 75, further comprising determining the sequences of the barcoded nucleic acid molecules from the enriched subset to resolve the heterogeneity in the infusion product.
